(19) United States
(12) Patent Application Publication
(10) Pub. No.: US 2016/0319354 A1
(43) Pub. Date: Nov. 3, 2016

(54) SYSTEMS, COMPOSITIONS AND METHODS FOR DETECTING AND ANALYZING MICRO-RNA PROFILES FROM A BIOLOGICAL SAMPLE

(71) Applicant: MIROCULUS INC., San Francisco, CA (US)

(72) Inventors: Alejandro TOCIGL, Mountain View, CA (US); Foteini CHRISTODOULOU, Athens (GR); Pablo OLIVARES, Santiago (CH); Ferran GALINDO, Cuidad de Panama (PA); Jorge SOTO, Mexico City (MX)

(73) Assignee: MIROCULUS INC., San Francisco, CA (US)

(21) Appl. No.: 15/108,475

(22) PCT Filed: Dec. 30, 2014
(86) PCT No.: PCT/US14/72802

§ 371 Date: Jun. 27, 2016

Related U.S. Application Data
(60) Provisional application No. 61/921,761, filed on Dec. 30, 2013.
(60) Provisional application No. 62/068,589, filed on Oct. 24, 2014.

(51) Int. Cl. C12Q 1/68; G01N 35/00; B01L 7/00

(52) U.S. Cl. CPC C12Q 1/6883; C12Q 1/6886; B01L 7/00; G01N 35/00871; C12Q 2600/16; C12Q 2600/158; C12Q 2600/178; B01L 2300/0829; B01L 2300/18; B01L 2300/0654; B01L 2300/023; B01L 2300/027; G01N 2035/00891; G01N 2035/009; C12Q 2600/118

(57) ABSTRACT

Methods and apparatuses for detecting microRNA from a tissue sample. In particular, described herein are multiplexed methods and apparatuses for implementing them for rapid and parallel detection of a profile of different microRNAs in a patient sample using a modified loop-mediated isothermal amplification (“LAMP”) technique.

### CROSS REFERENCE TO RELATED APPLICATIONS

This patent application claims priority to each of the following U.S. provisional patent applications: U.S. provisional patent application No. 61/921,761, filed on Dec. 30, 2013 (titled “DEVICE AND METHOD FOR DETECTING AND ANALYZING A SMALL RNA PROFILE FROM A BIOLOGICAL SAMPLE”), and U.S. provisional patent application No. 62/068,589, filed on Oct. 24, 2014 (titled “COMPOSITIONS AND METHODS FOR DETECTING AND ANALYZING A SMALL RNA PROFILE FROM A BIOLOGICAL SAMPLE”). Each of these applications is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This invention relates to apparatuses (including systems and devices), compositions, kits, and methods for detecting small RNAs, especially microRNAs (miRNAs).

### BACKGROUND

MicroRNAs (miRNAs) are small (typically 18-25 nucleotides) non-coding RNAs that are important in regulating gene expression by binding to mRNA transcripts and influencing their stability or translation efficiency. MiRNAs have been shown to circulate within blood and appear to be relatively stable in the plasma and serum. Recently, miRNA expression profiles in certain cancers and diseases have been found to be altered, suggesting that some miRNAs, individually or as miRNA signatures, can be used as diagnostic and/or prognostic biomarkers, and/or as biomarkers to monitor responses to therapeutic interventions.

For example, there may be significant overexpression of miR-141 in individuals with prostate cancer compared with normal individuals. At a miRNA-141 level of above 2,500 copies per μl of serum, individuals with prostate cancer have been identified with 100% clinical specificity and 60%>clinical sensitivity. MiRNA levels in individuals diagnosed with cancer have been shown to be moderately correlated with their PSA levels with Pearson and Spearman (rank) correlation coefficients of +0.85 and +0.62. Numerous other correlations between microRNAs and disease states have been suggested.

To date, various nucleic acid assay technologies have been used to identify and characterize miRNAs, such as microarray- and polymerase chain reaction (PCR)-based assays. Particularly for miRNAs that are present in low amounts, amplification techniques such as quantitative real-time reverse transcriptase polymerase chain reaction (qRT-PCR) or isothermal NASBA, (nucleic acid sequence based amplification) has been used to amplify the targets of interest. However, immobilization of probes or target amplification decreases assay sensitivities and increases cost and time requirements. Currently proposed methods are less effective and may be difficult to reliably interpret.

A plethora of research in the past 10 years provides solid evidence that microRNA expression profiling can distinguish subtypes of cancer, even their stages, in much higher accuracy than genomics, transcriptomics or proteomics analyses. Recently, microRNAs have been found deregulated in the bloodstream, accurately reflecting a wide range of physiopathology including many types of cancer, metabolic, psychiatric and cardiovascular diseases.

Adaptations of pre-existing molecular profiling methods (such as microarray, qPCR and massive parallel sequencing) to detect small RNAs and in particular miRNAs in tissues, cells and biofluids have been proposed. However, the chemistries available up to date are not affordable for routine check-up diagnostic purpose (at least to the majority of people who prefer to pay rent over a diagnostic test) nor are the instruments necessary to collect the data (with the most affordable costing around $20,000 USD). Thus, high investment costs hamper the growth of global miRNA market. In addition, lack of skilled professionals also obstructs the growth of global miRNA market.

Described herein are innovative systems, including apparatuses (device and methods) and biochemistries that may substantially reduce the cost of miRNA assays compared to existing device, and may allow for usage of simpler yet equally accurate instrumentation capable of documenting and uploading data to cloud servers where analysis, interpretation and other contextual data gathering may take place. Aside from cost, the chemistry described herein may be simpler yet accurate enabling the screen for many miRNAs to happen simultaneously during the span of 60 to 90 minutes. The assays for miRNAs in biofluids described herein are affordable, fast and non-invasive.

Therefore, there is a critical need for a method that allows fast, sensitive, and highly reproducible (as well as inexpensive) detection of microRNAs from a crude, unpurified sample where target concentrations may be very low. Described herein are method and system that may address this need.

### SUMMARY OF THE DISCLOSURE

In general, described herein are methods and apparatuses for detecting microRNA from a tissue sample. In particular, described herein are multiplexed methods and apparatuses for implementing them for rapid and parallel detection of a profile of different microRNAs in a patient sample using a modified loop-mediated isothermal amplification (“LAMP”) technique. Although the LAMP technique is known, the methods described herein provide a novel and an unexpectedly effective modification of traditional LAMP methods, including RNA-based LAMP methods. Although a brief summary of LAMP is provided herein, this description uses terminology and presumes familiarity with LAMP techniques and nucleic acid handling techniques, including at least: Notomi et al. (“Loop-mediated isothermal amplification of DNA”) Nucl. Acid Res. 28(12): e63 (2000); and also Maroney et al. (“Direct detection of small RNAs using splinted ligation”) Nat. Protocols, vol. 3, no. 2 (2008), pp. 279-287; Nilsson et al., (“RNA-templated DNA ligation for transcript analysis”), Nucl. Acid Res. 29(2) (2001), pp. 578-581; Maroney et al. (“A Rapid, quantitative assay for direct detection of microRNAs and other small RNAs using splinted ligation”) RNA (2007), 13:930-936.

Part I of this application describes the detection of microRNAs by the generation of LAMP template DNA only in presence of target miRNA. In particular, described herein is the use of partial templates for LAMP that may be joined by splinting with target microRNAs to form a template from which LAMP may be occur; the template may include specific nucleotide regions that uniquely identify the microRNA used for the template and allow downstream specific amplification by LAMP even in the presence of a large number of different microRNA templates. This may allow multiplexed detection using LAMP, which has previously proven difficult to reliably achieve. The methods and systems described herein are adapted for reliable detection and in particular may provide a highly reproducible qualitative assay.

Part II of this application describes method and apparatuses for the detection of specific microRNAs by the generation of LAMP inner primers only in presence of a target miRNA, in which one or both inner primers for LAMP are produced in vitro prior to the LAMP reaction. Their generation is conditional and specific to the presence of target miRNAs.

In both cases, the absence of miRNA from biological sample (or the lack of a certain concentration of microRNAs below a predetermined threshold) will mean absence of one or both inner primers for LAMP and therefore no amplification and fluorescence signal will be produced by the assay.

Part III of this description describes and illustrates apparatuses, including systems and devices, assays and kits that may be used to perform the methods described in parts I or II. In particular, described herein are low-cost, portable and easy to use apparatus for the detection of microRNA from a patient sample.

For example, embodiments of the method and apparatuses described herein may include a method of detecting micro RNA (miRNA) comprising distributing RNA or cDNA obtained from a biological sample into a plurality of discrete reaction wells comprising a distinct probe (e.g., primers) targeting a template specific to a particular miRNA (even where the probes are not directed to the microRNA sequence itself), and probes and reagents to perform an isothermal amplification assay, forming a plurality of distinct reaction mixtures, simultaneously performing isothermal amplification with each of the reaction mixtures, wherein amplification of a product in a reaction well indicates the presence of the corresponding miRNA.

Embodiments may also include a method of generating a miRNA expression comparison report comprising distributing RNA or cDNA obtained from a biological sample into a plurality of discrete reaction wells, each of the wells targeting a particular (predetermined) miRNA, and probes and reagents to perform an isothermal amplification assay, forming a plurality of distinct reaction mixtures, simultaneously performing the isothermal amplification assay with each of the reaction mixtures, obtaining optical data (including an image of the reaction wells) to detect amplification products from the amplification assay or record readings/data through spectrophotometry or other optical means, transmitting the image or numerical data to a system for analyzing expression data, the system comprising one or more processors configured to receive input of the image or numeric data, store a database comprising miRNA expression profiles correlating with a plurality of diseases, store software for performing comparisons of the expression profiles in the database with the expression profile obtained from the biological sample, and memory coupled to the one or more processors, configured to provide the processor with instructions, and running the software to generate a miRNA expression comparison report.

Also described are methods of detecting and analyzing a micro-RNA profile in a biologic fluid, comprising preparing a multiwell plate or microfluidic chip with a sample of biologic fluid (e.g., after first performing a combined, total microRNA template-preparing step as described herein), the plate or microfluidic chip having multiple chambers, each chamber being configured to target a specific micro-RNA, utilizing the LAMP method to cause a fluorescence or color profile of the plate, the fluorescence profile indicating the presence or absence of specific miRNAs in each of the multiple chambers, detecting the optical (e.g., fluorescence) profile with a detector, transmitting the optical profile to a server, and analyzing the optical profile, by the server, to associate the received profile with one or more diseases or health-related conditions.

As mentioned, also described herein are partially or fully enclosed devices comprising an opening for ingress and egress of a multiwell reaction substrate, a thermal control portion capable of maintaining a select temperature and for receiving a multiwell reaction substrate; and a detector. In some variations the detector may be dedicated illumination and/or optical sensors; in some variations the detector may be a separate detector, including, e.g., a camera of a smartphone that operates with the device, and is aligned with the surface for receiving the multiwell reaction substrate. The thermal control portion may include a thermal block, or in some variations, may not include a separate thermal block, but may include circuitry on a circuit board that is adapted to directly surround some or all of the wells of the multiwell reaction substrate, allowing direct and inexpensive control of the temperature for the LAMP procedure.

In some variations the apparatus may also include one or more large-scale wells for performing the grouped template-forming steps described herein; for example, a large chamber may be provided for regulating the temperature of the material undergoing the bulk ligation procedure (e.g., a multiplexing mixture) forming the templates for all of the microRNAs to be detected. The apparatus may also include a timer and or output to help guide the user in preparing the multiplexing mixture.

Any embodiment of a method or composition described herein can be implemented with respect to any other method or composition described herein.

In general, described herein are methods of detecting a plurality of microRNAs in parallel from a patient sample containing microRNA using a multiplexed ligation and detection technique. For example a method may include the steps of: combining the patient sample with a first mixture to form a multiplexing mixture comprising a plurality of pairs of donor template and acceptor template, wherein each pair of donor template and acceptor template is specific to a target microRNA of the plurality of microRNAs because at least one of the 5′ end of a donor template and 3′ end of an acceptor template in each pair comprise a region that is complimentary to adjacent portions of the target microRNA, and further wherein one or both of the 3′ end of the donor template and the 5′ end of the acceptor template comprises one or more nucleotide sequence that is specific to the pair of donor and acceptor template; heating the multiplexing mixture to denature the microRNA, and cooling the multiplexing mixture to anneal pairs of donor and acceptor template to specific target microRNA if the target microRNA is present in the multiplexing mixture; ligating the annealed pairs of donor template and acceptor template using a ligase (e.g. DNA ligase) to form a template specific to target microRNA; inactivating the ligase; placing a portion of the multiplexing mixture into each of a plurality of wells; performing, in parallel, loop-mediated isothermal amplification of template specific to a different target microRNA in each of the plurality of wells, wherein each well is associated with one specific target microRNA from the plurality of microRNAs and wherein each well comprises a polymerase having strand displacement activity and primers for the loop mediated amplification, wherein one or more of the primers for loop mediated amplification includes the nucleotide sequence that is specific to the pair of donor and acceptor template or a complement to the nucleotide sequence that is specific to the pair of donor and acceptor template and therefore specific to one target microRNA of the plurality of microRNAs.

Any of these method of detecting a plurality of microRNAs in parallel from a patient sample containing microRNA using a multiplexed ligation and detection technique may also include: combining the patient sample with a first mixture to form a multiplexing mixture comprising a plurality of pairs of donor template and acceptor template, wherein a 5′ end of the donor template and a 3′ end of the acceptor template of each pair comprise regions that are complimentary to adjacent portions of one specific target microRNA from the plurality of microRNAs, and further wherein each acceptor template comprises a B3 region at a 5′ end of the acceptor template, a B2 region 3′ to the B3 region, and a B1 region 3′ to the B2 region, wherein each donor template comprises an F3c region at the 3′ end of the donor template, an F2c region 5′ to the F3c region, and an F1c region 5′ to the F2c region, and wherein each pair of donor and acceptor templates includes a unique sequence that is different from the other pairs for at least one of: the B3 region, the B2 region, the B1 region, the F3c region, the F2c region, and the F1c region; heating the multiplexing mixture to denature the microRNA, and cooling the multiplexing mixture to anneal the pair of donor and acceptor template to the specific target microRNA if that specific target microRNA is present in the multiplexing mixture; ligating the annealed pairs of donor template and acceptor template using a ligase to form a template specific to target microRNA; inactivating the ligase; placing a portion of the multiplexing mixture into each of a plurality of wells; performing loop-mediated isothermal amplification of each of the plurality of wells in parallel, wherein each well is associated with one specific target microRNA from the plurality of microRNAs and comprises a combination of primers that complement or include the unique sequence that is different from the other pairs of the plurality of pairs of donor template and acceptor template for at least one of: the B3 region, the B2 region, the B1 region, the F3c region, the F2c region, and the F1c region of the template specific to target microRNA.

In some variations, these methods include a step of forming a bridging oligo using a detection oligo that comprises a sequence complementary to the target microRNA at one end (e.g., a 5′ end) that is connected to reverse complement of a DNA oligo. The DNA oligo may be combined with the patient sample RNA (or a sample including RNA from the patient sample) and the hybrid bridging oligo may be used to splint the target RNA and DNA oligo to form an RNA-DNA splint oligo that is used in the methods described herein, where the target and acceptor templates for the LAMP portion of the assay are configured with a complement to a portion of the RNA-DNA splint oligo (e.g. the DNA olio) on the donor template and an adjacent portion of a compliment to the RNA-DNA oligo (e.g., a compliment to the target RNA) on the acceptor template. In this variation one of the target and acceptor templates (e.g., the donor) may be ‘generic’ and used with any of the target microRNA-specific acceptor templates to form the whole template for LAMP amplification.

In some variations, for each specific target microRNA, the donor template in one of the plurality of pairs comprises a reverse compliment at its 5′ end of a first portion of the specific target microRNA sequence and wherein the acceptor template in each pair comprises a reverse compliment at its 3′ end of a second portion of the specific target microRNA sequence. The donor template of each pair may be modified to have a phosphate group at its 5′ end.

The donor template and the acceptor template of each pair may be relatively small oligonucleotides (e.g., having a length of less than 150 base pairs each, less than 140 bp, less than 130 bp, less than 120 bp, less than 110 bp, less than 100 bp, etc.).

The concentration of donor and acceptor template may be optimized for the reaction. For example, the step of combining may comprise combining the patient sample with the first mixture so that there is 10 nM or less of each of donor template and target template.

In general, each pair of donor and acceptor templates may include a unique sequence for the F2c region, the F1c region or both the F2c region and the F1c region.

Heating (e.g., to get ssDNA/ssRNA) may comprise heating the multiplexing mixture to between about 70° C. and 99° C. for greater than 1 min. Cooling the multiplexing mixture may comprise gradually cooling to room temperature. The ligation components may also be optimized. For example, ligation may comprise adding less than 4 nM of ligase into the multiplexing mixture. Ligating may comprise using less than 4 nM of ligase in the presence of MnCl2 and less than 5 μM ATP in the multiplexing mixture. Ligating may comprise ligating for between about 10-60 min at between about 20-40° C. In some variations the ligase may be inactivated by heating the multiplexing mixture to greater than 60° C. for 10 min or more

In some variations, performing loop-mediated isothermal amplification (LAMP) comprises amplifying one of the templates specific to target microRNA in each well to indicate the presence of the target microRNA in the patient sample by maintaining the temperature of the well between 60 and 70 degrees in the presence of a forward inner primer (FIP) that hybridizes to the nucleotide sequence that is specific to the pair of donor and acceptor template specific to target microRNA and includes a second region that is identical to a portion of the template specific to target microRNA. Further, performing loop-mediated isothermal amplification further may comprise amplifying in the presence of a forward outer primer (FOP) that hybridizes to the template specific to target microRNA, a backwards inner primer (BIP) comprising a nucleotide region of the template specific to target microRNA and a second region that hybridizes to the template specific to target microRNA, and a backwards outer primer (BOP) comprising a region of the template specific to target microRNA.

For example, performing loop-mediated isothermal amplification may comprise amplifying one of the templates specific to target microRNA in each well to indicate the presence of the target microRNA in the patient sample by maintaining the temperature of the well between 60 and 70 degrees in the presence of a forward inner primer (FIP) comprising an F2 region that hybridizes to the F2c region of the template specific to target microRNA and the F1c region of the template specific to target microRNA, a forward outer primer (FOP) comprising an F3 region that hybridizes to the F3c region of the template specific to target microRNA, a backwards inner primer (BIP) comprising the B2 region of the template specific to target microRNA and a B1c region that hybridizes to the B1 region of the template specific to target microRNA, and a backwards outer primer (BOP) comprising the B3 region of the template specific to target microRNA, and a polymerase having strand displacement activity.

Any of the methods described herein may also include detecting a visual change in one or more wells indicating the presence of the specific target microRNA associated with that well in the patient sample. Further, any of these methods may also include correlating signals corresponding to a visual change in a plurality of the wells with known profiles corresponding to disease states to identify a disease state, condition, and/or disorder, and/or transmitting a signal corresponding to a visual change in plurality of the wells to a remote processor for correlation analysis with known profiles corresponding to disease states.

Apparatuses for performing any of the methods or components of the methods are also described herein. For example, described herein are systems for detecting a plurality of microRNAs in parallel from a patient sample containing microRNA using a multiplexed ligation and detection technique. An exemplary system may include the multiplexing mixture (with the partial templates to be joined in the presence of the proper specific microRNA) and multiwell plate (e.g., multiwell reaction substrate) for performing the LAMP assay, which may be pre-loaded with some of the LAMP assay components. A device for controlling the temperature and/or monitoring the assay development may also be included as part of the system, as may software (e.g., an application) for controlling the device and assisting with performance of the assay. For example, an exemplary system may include: a first solution mixture comprising a plurality of pairs of donor template and acceptor template, wherein each pair of donor template and acceptor template is specific to a target microRNA of the plurality of microRNAs because a 5′ end of a donor template and a 3′ end of an acceptor template in each pair comprise regions that are complimentary to adjacent portions of the target microRNA and further wherein one or both of the 3′ end of the donor template and the 5′ end of the acceptor template comprises one or more nucleotide sequence that is specific to the pair of donor and acceptor template; and a multiwell reaction substrate for performing, in parallel, loop-mediated isothermal amplification to detect target microRNA in each of a plurality of wells, wherein each well is associated with one specific target microRNA from the plurality of microRNAs, and wherein each well comprises a plurality of primers for the loop mediated amplification, wherein one or more of the primers for loop mediated amplification within each well includes the nucleotide sequence that is specific to the pair of donor and acceptor template or a complement to the nucleotide sequence that is specific to the pair of donor and acceptor template and therefore specific to one target microRNA of the plurality of microRNAs associated with that well.

The multiwell reaction substrate may include a polymerase having strand displacement activity within each well.

As mentioned, any of these systems may also include a multiwell plate reader for performing, in parallel, loop-mediated isothermal amplification to detect target microRNA in each of a plurality of wells of a multiwell reaction substrate. For example, the reader may be configured so that each well is associated with one specific target microRNA from the plurality of microRNAs. The multiwell plate reader may include: thermal control circuitry configured to maintain the plurality of wells at a temperature of between 60-70° C., wherein the control circuitry comprises a board having a plurality of thermal control elements configured to surround individual wells of the multiwell reaction substrate, one or more light sources configured to illuminate wells of the multiwell reaction substrate, a plurality of optical detectors, wherein each optical detector is configured to monitor a well of the multiwell reaction substrate, and a communication module configured to transmit sample data collected from the plurality of optical detectors to a remote processor.

In some variations the first solution mixture (e.g., preloaded into a reaction vessel) may be lyophilized. Similarly the multiwell plate may be pre-loaded with lyophilized components for the LAMP assay(s).

Any of the systems for detecting a plurality of microRNAs in parallel from a patient sample containing microRNA using a multiplexed ligation and detection technique described herein may include: a first solution mixture comprising a plurality of pairs of donor template and acceptor template, wherein each pair of donor template and acceptor template is specific to a target microRNA of the plurality of microRNAs because a 5′ end of a donor template and a 3′ end of an acceptor template in each pair comprise regions that are complimentary to adjacent portions of the target microRNA and further wherein one or both of the 3′ end of the donor template and the 5′ end of the acceptor template comprises one or more nucleotide sequence that is specific to the pair of donor and acceptor template; and a multiwell plate reader for performing, in parallel, loop-mediated isothermal amplification to detect target microRNA in each of a plurality of wells of a multiwell reaction substrate, wherein each well is associated with one specific target microRNA from the plurality of microRNAs, the multiwell plate reader comprising: thermal control circuitry configured to maintain the plurality of wells at a temperature of between 60-70° C., wherein the control circuitry comprises a board having a plurality of thermal control elements configured to surround individual wells of the multiwell reaction substrate, one or more light sources configured to illuminate wells of the multiwell reaction substrate, a plurality of optical detectors, wherein each optical detector is configured to monitor a well of the multiwell reaction substrate, and a communication module configured to transmit sample data collected from the plurality of optical detectors to a remote processor.

As mentioned above, any of these systems may include a multiwell reaction substrate (which may be pre-loaded with any of the components for the assay, e.g., in concentrated and/or lyophilized form). Further, the first solution mixture is lyophilized.

In general, any of the devices (e.g., plate reader devices) may include a communication module that is a wireless communication module (e.g., Bluetooth).

Any of the systems described herein may also include control logic (e.g., software, such as an application) that comprises a non-transitory computer-readable storage medium storing a set of instructions capable of being executed by a processor (e.g., on a computer such as a handheld device, e.g., smartphone) to control the operation of the multiwell plate reader, and that when executed by the smartphone, causes the smartphone to: identify and wirelessly communicate with the multiwell plate reader; associate a multiwell reaction substrate with a patient; start a detection assay in the multiwell plate reader; receive optical data from the multiwell plate reader, wherein the optical data comprises optical information from the plurality of optical detectors; and connect to a remote server to transmit and receive information about the optical data.

The set of instructions, when executed by the smartphone, may cause the processor (e.g., smartphone) to transmit an alert when the detection assay is completed. The set of instructions when executed by the processor may causes the processor to save data for later transmission to the remote server, and/or present information about the optical data on a display of the smartphone, and/or receive optical data from the multiwell plate reader at periodic intervals for a predetermined period of time.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A schematically illustrates one example of a pair of templates, an acceptor and donor template (oligonucleotide) for annealing to an miRNA of interest to from a specific LAMP template as described herein.

FIGS. 1B-1D schematically illustrate a multiplexed ligation method to form the specific LAMP templates for parallel detection, as described herein. In FIG. 1B, a plurality of donor (on left) and acceptor (on right) templates are schematically shown. For convenience, only a single pair of donor and acceptor templates is included for a particular micro RNA. In FIG. 1C, the multiplexing mixture is schematically illustrated, including a mixture of donor and acceptor template portions directed to specific target microRNAs; as shown by the arrow, a patient sample having total RNA (and therefore including microRNAs) may be added to the multiplexing mixture. FIG. 1D schematically illustrates splinting of target microRNAs to the specific donor and acceptor templates by hybridization, following by ligation (e.g., using a T4 DNA ligase) to form the specific LAMP templates for target microRNAs present in the sample.

FIG. 1E schematically illustrates LAMP templates and primers for an exemplary method of detecting microRNAs from a patient sample following-up on the illustrations of FIGS. 1B-1D. From the patient sample that was assayed by the multiplexing reaction shown in FIG. 1C-1D, five microRNAs (MiRNA(1), MiRNA(2), MiRNA(3), MiRNA(4), MiRNA(5)) were detected by the splinting hybridization and ligated to form template for LAMP amplification. The resulting templates include at least one distinct region (in this example, in the F1c region of the donor template portion) that can be used to selectively amplify the template corresponding to individual microRNAs by use of the specific LAMP primer directed to this region (e.g., the FIP); the remaining LAMP primers are general or generic to all of the templates, as shown.

FIG. 2 illustrates an another variation of a multiplexing method that includes two ligation steps, rather than one, in order to form the template for LAMP.

FIG. 3 shows results from assaying for miRNA using a multiplexed microRNA detection assay as described herein with a high ratio of ligase (e.g., SplintR) enzyme to oligonucleotide.

FIG. 4 shows results from performing a multiplexed microRNA detection assay as described herein with ligation reaction using T4 (DNA) ligase.

FIG. 5 shows results from performing a multiplexed microRNA detection assay as described herein for miR-1 muscle specific miRNA in various tissues using a multiplexed microRNA detection assay as described herein.

FIG. 6 shows results from assaying for miR-122 liver specific miRNA in various tissues using a multiplexed microRNA detection assay as described herein.

FIG. 7 shows results from assaying for miR124 brain specific miRNA in various tissues using a multiplexed microRNA detection assay as described herein.

FIG. 8 shows results from assaying for miR-16 marker for haemolysed plasma in various samples using a multiplexed microRNA detection assay as described herein; the inset in FIG. 8 shows the raw results for exemplary samples, which were optically read.

FIG. 9 schematically illustrates the detection of specific microRNAs by the generation of LAMP inner primers only in presence of a target miRNA in which an RNA splinteded ligation step is included prior to amplification using LAMP.

FIG. 10 schematically illustrates the detection of specific microRNAs by the generation of LAMP inner primers only in presence of a target miRNA in which a DNA splinteded ligation step is included prior to amplification using LAMP.

FIGS. 11A-11C illustrates one variation of a prototype device which hosts the miRNA assay system while sending information and results of each sample to a remote server. FIG. 11A shows a front perspective view of one configuration of a plate-reader device for performing at least the LAMP portion of the assay as described herein, in an closed (left) and open (right) configuration. FIG. 11B is an enlarged view of the exemplary plate reader apparatus of FIG. 11A. FIG. 11C is a section through the exemplary device shown in FIGS. 11A-11B.

FIG. 11D is another variation of a device for detecting a micro-RNA profile in a biologic fluid, in accordance with a first exemplary embodiment of the present disclosure.

FIG. 12 is a flowchart illustrating some operational features of an apparatus and for detecting and analyzing a patient's microRNA profile.

FIG. 13 is a schematic diagram illustrating one variation of a circuit board layout for a shield element integrating multiple features and controlled by a processor which may form part of an apparatus as described herein.

FIG. 14 is a schematic illustration of one variation of the integrating shield component of FIG. 13.

FIG. 15 shows one example of a prototype shield for a variation of an apparatus for detecting microRNAs as described herein.

FIG. 16 is a schematic diagram illustrating one variation of a circuit board layout for a temperature control (e.g., heater) board that may be used as part of an apparatus for detecting microRNAs as described herein.

FIG. 17 is a schematic illustration of one variation of the temperature-controlling board of FIG. 16.

FIG. 18 is a prototype of one variation of the temperature-controlling board of FIGS. 16-17.

FIG. 19 is a schematic diagram illustrating one variation of a circuit board layout for a sensor board (illumination/detection) that may be used as part of an apparatus for detecting microRNAs as described herein.

FIG. 20 is a schematic illustration of one variation of the sensor board of FIG. 19.

FIGS. 21 and 22 show front and back views of a prototype of one variation of a sensor board that may be used as part of an apparatus for detecting microRNAs as described herein.

FIG. 23 is a flowchart illustrating one variation of a test flow for detecting, analyzing and reporting a patient's microRNA profile and/or a diagnosis based on the profile.

FIG. 24 is a flowchart illustrating the analysis and operations that may be performed on a patient's microRNA profile in a remote server.

FIG. 25 is a chart illustrating processes that may be performed for detecting and analysis of a microRNA profile from a biologic fluid.

FIGS. 26A-26C are variations of a diagram illustrating associations between micro-RNAs and certain diseases or disorders (e.g., cancers).

FIG. 27 depicts a flowchart of one variations of a method of microRNA detection using the methods and apparatuses described herein.

### DETAILED DESCRIPTION

Described herein are apparatuses (including device and methods), compositions, kits, and methods for detecting and analyzing a small RNA. Such detection and analysis may be useful for diagnosing, prognosticating (e.g. predicting a risk for getting), or treating a disease or syndrome, or analyzing response to a disease treatment, or performing another analysis.

Small RNAs called microRNAs (or miRNA) are produced from DNA found in animal and plant cells and in viruses. MiRNAs are small (e.g., about 22 nucleotides) non-coding RNA molecules. Hundreds of such microRNAs have been identified and sequenced thus far. They have been shown to be involved in various biological processes such as gene expression and post-transcriptional modification. The action of a single miRNA may have an effect on dozens of different genes, RNAs, or proteins. MiRNA expression is thought to reflect the health of a person and knowing miRNA expression may be especially useful for determining a health or disease status of a person (or another other organism). Their expression may be helpful in diagnosing, prognosticating (e.g. predicting a risk for getting), or treating a disease or syndrome, or analyzing response to a disease treatment, or performing another analysis. In some cases, it is desirable that a health care profession or technician analyze an expression of microRNAs (e.g., from a blood or other patient sample) very quickly, such as at the time the patient is being treated, referred to as the point of care (POC). In such cases, health care decisions may be quickly made and implemented. Although a having an assay that gives results quickly in order to provide results useful at the time of care (e.g., an assay that will detect a small miRNA quickly) is a minimum requirement, it is not sufficient. In addition, such an assay should, ideally, be relatively easy to perform and not require extensive training of personnel or complicated machinery to perform. An assay should, ideally, be cost effective and minimize cost per assay. Finally, such an assay should be both specific and sensitive as a false positive or a missed diagnosis may have significant negative consequences and could result in delayed or unnecessary treatment that could be costly or dangerous. It is highly challenging to provide an assay that can meet some or even most of these goals. In particular, even a seemingly small improvement in any of these or other metrics for an assay for making health care or other decisions is highly desirable.

In the past, performing an RNA assay has generally required expensive reagents, specialized and expensive equipment, extensive technician training, and a significant amount of time, making them less than desirable as quick and reliable assays. Improvements in one metric often meant comprising on another one. For example, while it is less expensive (at least initially) to perform an assay using a very small patient sample and relatively smaller amounts of expensive reagents, stochastic (random) events begin to become important and skew the results, making the assay less reliable. For example, reverse transcription polymerase chain reaction (RT-PCR) which is commonly used to analyze RNA has been notorious for creating artifacts, and much care has gone into improving its reliability, often at the expense of comprising other desirable factors. For example, one improvement that reduces random events with RT-PCR and its associated problems, was the development of digital PCR, which involves dividing a sample into multiple portions and performing a PCR reaction on each portion. Although the reliability of RT-PCR was improved, the assay became considerably more complex.

Described herein are apparatuses, compositions, kits and methods for detecting and analyzing small RNAs such as miRNAs although any RNA may be detected or analyzed. The apparatuses, compositions, kits, and methods provide improved assays such as having greater sensitivity, greater specificity, less background, lower cost, etc. The assays described herein take advantage of desirable properties of enzymes that are able to recognize discontinuous pieces of DNA that are hybridized to an RNA molecule and to ligate the pieces of DNA to form a single piece of DNA that can be readily be amplified. The assays also take advantage of an amplification procedure utilizing a simple isothermal procedure, loop-mediated isothermal amplification procedure to quickly amplify the DNA.

As used herein, “amplify” in reference to a nucleic acid sequence refers to increasing the number of copies of a nucleic acid sequence.

As used herein, “nucleic acid sequence” refers to a string of nucleotide bases attached by phosphodiester bonds, for example DNA has deoxynucleotides, i.e. combinations of adenine (A), guanine (G), cytosine (C), and thymine (T) molecules attached by covalent phosphodiester bonds, RNA, mRNA, and microRNA has ribonucleotide, i.e. combinations of adenine (A), guanine (G), cytosine (C), and uracil (U) nucleotide molecules attached by covalent phosphodiester bonds.

As used herein, “hand held” or “handheld” in reference to an electronic device refers to having the capability to be operated while being held in human hands, such that a hand held device is lightweight and/or small in size. Examples of “hand held” or “handheld” devices include a video camera, a laptop computer, a net-book, a tablet, a smart device, cell phone, a personal digital assistant (PDA), and the like. In particular, handheld may refer to a smart phone such as an iPhone™, Android™ phone, or the like. A smartphone may generally refer to wireless handheld device that includes one or more processors and may be used to transmit and/or receive information by one or more means, including Bluetooth, ultrasound, Zigbee and Ultra Wideband (UWB), etc. A “smartphone” may refer to an electronic device that can be cordless (unless while being charged), mobile (easily transportable) and capable of connecting to wireless receivers such as Wi-Fi, 3G, 4G, Bluetooth etc., including but not limited to devices such as a Blackberry, iPad, iPod Touch, iPod, iPhone, Droid, Android-based devices, etc. In some embodiments, a smart device functions as a processor.

As used herein, “optical detector” or “optical set up” refers to components that when used together provide an optical pathway for movement of optical energy, for example, beginning with an LED that emits optical energy, an optical fiber for capturing and transmitting optical energy, and a light energy collector for detecting optical energy for further analysis.

As used herein, “optical pathway” refers to the movement of optical energy (or light energy) through an optical system, for example, a continuous light pathway where optical energy moves from one end of the pathway to another end. One example of an optical pathway includes optical energy emitted by an LED that activates a fluorescent molecule that in turn emits optical energy that is captured by one end of an optical fiber for transmission to the other end of the optical fiber where the optical energy optionally passes through an emission filter for detection by a photodiode. As another example, light energy emitted from an LED light source travels through a sample well area into a sample well and is absorbed by a fluorescent molecule, and the fluorescent molecule emits light energy which is captured by an optical fiber which in turns allows transmission of the light energy to a light energy collector, such as a photodiode (PD).

As used herein, “battery power” in reference to a power supply for an electronic device refers to obtaining electrical energy from a battery in the form of DC. In general, any of the apparatuses described herein may be battery powered and/or wall powered.

As used herein, the term “processor” refers to a device that performs a set of steps according to a program (e.g., a digital computer). Processors, for example, include Central Processing Units (“CPUs”), small CPUs such as microcontrollers, electronic devices, and systems for receiving, transmitting, storing and/or manipulating digital data under programmed control.

As used herein, the term “target” in reference to an amplified nucleic acid sequence (e.g., microRNA) may refer to the source or original nucleic acid in a sample, such that when an amplified nucleic acid sequence is detected by the devices and methods described herein the target is found in the sample. For example, a particular microorganism can have a target nucleic acid, which when detected by the devices and methods described herein signifies that the microorganism is present in the sample. As another example, the target can be a cancer marker, amplification of a nucleic acid encoding the cancer marker, identifies that the cancer marker is present in the sample.

As used herein, “electrical communication” in reference to electrical components refers to a conductive pathway (e.g., wire) attaching two or more components. As used herein, “wireless communication” or “wireless network” in reference to a device refers to the capability of a device to transmit information, such as the results obtaining from using a device of the present disclosure, without the use of a physical wire.

As used herein, “chamber” or “well” in reference to a sample, such as a biological sample chamber or sample well, refers to an area capable of holding a biological sample (and reagents such as primers) in a distinct area. A multiwell reaction substrate may refer to a plate or other apparatus having multiple wells.

As used herein, “light source” in reference to an illuminating (illumination) light source refers to an excitation light source for exciting electrons in a fluorescent molecule. As used herein, “detecting” in reference to an optical signal may include, but is not limited to light emitted by a fluorescent compound (e.g., sensing an optical signal emitted from the fluorescent compound). Detecting may also include detecting non-florescent, e.g., colorimetric, turbidometric, etc., signals.

As used herein, “light-emitting diode” or “LED” refers to a semiconductor device that when electrically stimulated emits a form of electroluminescence as optical energy. As used herein, “organic light-emitting diode” or “OLED” may refer to a light-emitting diode (LED) in which the emissive layer comprises a thin-film of organic compounds for emitting optical energy or “light”.

As used herein, a “microRNA” or “miRNA” typically refers to a ribonucleic acid (RNA) molecule, for one example, approximately 22 nucleotides in length. In one embodiment, miRNA sequences bind to complementary sequences in the 3′ UTR of target mRNAs, usually resulting in silencing of the target mRNA, so that the target mRNA is not translated.

As used herein, a “fluorescent molecule” or “fluorophore” or “fluorophores molecule” or “fluorescent dye” in general refers to a molecule capable of excitation, i.e. activation, under conditions for emitting an optical energy emission, i.e. signal, for example, synthetic dyes, orange fluorescent dyes (stain) having exemplary optimal excitation wavelengths (i.e. spectra) in the 530 nm to 570 nm range and exemplary emission wavelengths in the 545-583 nm range, such as orange SYTO® 81, SYTO®-82, and cyanine dyes, asymmetrical cyanine dyes, green fluorescent dyes (stain), such as SYBR® dyes, i.e., SYBR Green I and II, and green SYTO® dyes, etc. For the purposes of the present disclosure, a fluorescent molecule is capable of binding to a nucleic acid sequence. In some embodiments, the biological sample comprises a fluorescent compound, wherein the fluorescent compound is selected from the group consisting of SYBR™ Brilliant Green, SYBR™ Green I, SYBR™ Green II, SYBR™ gold, SYBR™ safe, EvaGreen™, a green fluorescent protein (GFP), fluorescein, ethidium bromide (EtBr), thiazole orange (TO), oxazole yellow (YO), thiarole orange (TOTO), oxazole yellow homodimer (YOYO), oxazole yellow homodimer (YOYO-1), SYPRO® Ruby, SYPRO® Orange, Coomassie Fluor™ Orange stains, and derivatives thereof. These dyes are generally available commercially, and many of them can be made as described by Deligeorgiev et al., Recent Pat. Mat. Sci. 2: 1-26 (2006).

As used herein, an “optically activated fluorescent molecule” “optically activated fluorescent molecule” refers to a fluorescent molecule illuminated (i.e. excitation) under conditions for releasing energy as emitted light (i.e. emission) measured spectrally as wavelengths i.e. spectral profiles. In other words, light comprising wavelengths capable of exciting a fluorescent molecule, i.e. excitation light, causing the molecule to release emission energy capable of detection, i.e. captured, using a device of the present disclosures.

As used herein, “optical signal” may refer to any energy (e.g., photo-detectable energy) emitted from a sample.

As used herein, the term “primer” may refer to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer may first be treated to separate its strands before being used to prepare extension products. A primer may be an oligodeoxyribonucleotide. The primer may be sufficiently long to prime the synthesis of extension products in the presence of the inducing agent. The exact lengths of the primers will depend on many factors, including temperature, source of primer and the use of the method. A primer may also be referred to as a probe.

As used herein, the term “probe” may refer to a molecule (e.g., an oligonucleotide, whether occurring naturally as in a purified restriction digest or produced synthetically, recombinantly or by PCR amplification), that is capable of hybridizing to another molecule of interest (e.g., another oligonucleotide). When probes are oligonucleotides they may be single-stranded or double-stranded. Probes may be useful in the detection, identification and isolation of particular targets (e.g., gene sequences).

As used herein, “conventional QPCR” and “QPCR” refer to “quantitative PCR,” that for the purposes of the present disclosure is a real-time PCR analysis, such as real-time PCR reactions that are performed by a Taqman® thermal cycling device and reaction assays by Applied Biosystems. As used herein, “conventional PCR” and “PCR” refer to a nonquantitative PCR reaction, such as those reactions that take place in a stand-alone PCR machine without a real-time fluorescent readout.

As used herein, “isothermal amplification” refers to an amplification step that proceeds at one temperature and does not require a thermocycling apparatus.

As used herein, a heating element may refer to any electronic heater (e.g., resistive heating elements) including but not limited to semiconductor materials used as heating elements. As used herein, the term “photodiode” or “PD” refers to a solid-state light detector type including, but not limited to PN, PIN, APD and CCD.

As used herein, the terms “memory device,” and “computer memory” refer to any data storage device that is readable by a computer, including, but not limited to, random access memory, hard disks, magnetic (e.g., floppy) disks, zip disks, compact discs, DVDs, magnetic tape, and the like.

As used herein, the terms “optical detector” and “photo-detector” may refer to a device that generates an output signal when exposed to optical energy. Thus, in its broadest sense, the term “optical detector system” refers devices for converting energy from one form to another for the purpose of measurement of a physical quantity and/or for information transfer. Optical detectors include but are not limited to photomultipliers and photodiodes, as well as fluorescence detectors.

As used herein, “semiconductor” refers to a material that is neither a good conductor of electricity (such as copper) nor a good insulator (such as rubber) used in providing miniaturized components for taking up less space, faster and requiring less energy than larger components. Examples of common semiconductor materials are silicon and germanium and the like. As used herein, the term “TTL” stands for Transistor-Transistor Logic, a family of digital logic chips that comprise gates, flip/flops, counters etc. The family uses zero Volt and five Volt signals to represent logical “0” and “1” respectively. As used herein a circuit board may refer to a rigid or flexible planar substrate onto which one or more circuit elements are attached.

As used herein, “battery” may refer to a device that stores chemical energy and makes it available in an electrical form. Batteries comprise electrochemical devices such as one or more galvanic cells, fuel cells or flow cell, examples include, lead acid, nickel cadmium, nickel metal hydride, lithium ion, lithium polymer, CMOS battery and the like. As used herein, “CMOS battery” refers to a battery that maintains the time, date, hard disk and other configuration settings in the CMOS memory.

As used herein, “electronic power supply” refers to an electronic device that produces a particular DC voltage or current from a source of electricity such as a battery or wall outlet whereas using a wall outlet requires a “power supply” for converting AC into DC.

As used herein, “power supply” or “power adaptor” refers to an electrical system that converts AC current from the wall outlet into the DC currents required by computer and electronic device circuitry. Any of the power supplies described herein may be electronic power supplies.

As used herein, the term “target,” when used in reference to microRNA may refer to the molecules (e.g., nucleic acid) to be detected. Thus, the “target” is sought to be sorted out from other molecules (e.g., nucleic acid sequences) or is to be identified as being present in a sample through its specific interaction.

The terms “sample” and “specimen” are used herein in their broadest sense, and may include a biological sample and an environmental sample. Patient samples may include all types of samples obtained from humans and other animals, including but not limited to, body fluids such as urine, blood, fecal matter, cerebrospinal fluid (CSF), semen, and saliva, as well as solid tissue. Biological samples may be animal, including human, fluid or tissue.

As used herein, the term “oligonucleotides” or “oligos” refers to short sequences of nucleotides. As used herein, the terms “thermal cycler” or “thermal cycler” refer to a programmable thermal cycling machine, such as a device for performing PCR.

As used herein, the term “amplification reagents” may refer to those reagents (such as DNA polymerase, deoxyribonucleotide triphosphates, buffer, etc.), necessary for nucleic acid sequence amplification.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural state or source. An isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell genome in proximity to neighboring genes; RNA sequences, such as a specific mRNA sequence encoding a specific protein, are found in the cell as a mixture with numerous other mRNAs that encode a multitude of proteins. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “coding region” when used in reference to a structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets that specify stop codons (i.e., TAA, TAG, and TGA).

As used herein the term “portion” when in reference to a nucleotide sequence or nucleic acid (as in “a portion of a given nucleotide sequence” or a “portion of a nucleic acid”) refers to fragments of that sequence or that nucleic acid. The fragments may range in size from four nucleotides to the entire nucleotide sequence or nucleic acid minus one nucleotide.

The term “gene” may refer to a nucleic acid (e.g., DNA) sequence that comprises coding sequences necessary for the production of a polypeptide or precursor. It is intended that the term encompass polypeptides encoded by a full length coding sequence, as well as any portion of the coding sequence, so long as the desired activity and/or functional properties (e.g., enzymatic activity, ligand binding, etc.) of the full-length or fragmented polypeptide are retained. The term also encompasses the coding region of a structural gene and the sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as “5′ untranslated sequences.” The sequences that are located 3′ (i.e., “downstream”) of the coding region and that are present on the mRNA are referred to as “3′ untranslated sequences.” The term “gene” encompasses both cDNA and genomic forms of a gene. A genomic form of a genetic clone contains the coding region interrupted with non-coding sequences termed “introns” or “intervening regions” or “intervening sequences.” A subset of gene is “virulence and marker” genes or VMGs that refers to genes associated with virulence or used as markers for any specific reason. Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

Nucleic acid (e.g., DNA) molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” and “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification and hybridization reactions.

Equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur restricted between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

As used herein, “amplification” in reference to a method or apparatus described herein typically refers to amplifying a template or target nucleic acid sequence comprising the steps of hybridizing an amplification nucleic acid, such as a primer, to its complementary target sequence or sample nucleic acid sequence, also termed template nucleic acid sequence, in the presence of amplification reagents, free nucleic acids, and a polymerase, for example a BST polymerase for loop-mediated isothermal amplification, which results in the duplication of said complementary nucleic acid sequence then repeating these steps until amplification is detected or stopped. Amplification may be detected by a device of the present disclosures as fluorescent molecules become incorporated into the amplifying sequence or amplified sequence, or by removal of an optical quenching element to allow optical detection. Amplification may have a start time or point and an end time or point. Amplification may be considered a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribonucleotide or deoxyribonucleotide) specificity. Template specificity is frequently described in terms of “target” specificity. Target sequences are “targets” in the sense that they are sought to be sorted out from other nucleic acid. Amplification techniques have been designed primarily for this sorting out.

As used herein, the term “template” may refer to a sequence (e.g., polynucleotide sequence) including a microRNA sequence originating from a sample that is analyzed for the presence of “target.”

As used herein, “multiplexed” may refer to the simultaneous and grouped processing of multiple (e.g., more than 2, more than 3, more than 4, more than 5, more than 6, more than 7, more than 8, more than 9, more than 10, more than 15, more than 20, more than 30, more than 40, more than 50, more than 60, more than 70, more than 80, more than 90, etc.) targets, such as microRNAs within a single pool or collection. Multiplexed processing as described herein may be performed in parallel within the same container, typically without interference between the various targets, e.g., microRNAs.

As used herein an acceptor template may be an acceptor template DNA sequence that includes a portion (e.g., one half) of a template to be amplified by, e.g., LAMP. An acceptor template may include, for example, a 5′ region that includes three or more distinct regions of nucleotide sequences (e.g., B1, B2, B3 regions) used to form primers for LAMP amplification; a distinct 3′ region may include a portion (e.g., approximately half) of a target microRNA.

As used herein a donor template may be an acceptor template pDNA sequence (phosphorylated at the 5′ end) that includes a portion (e.g., approximately one half) of the microRNA template to be amplified by LAMP. A donor template may include, for example, a 3′ region that includes three or more distinct (non-overlapping) regions of nucleotide sequences (e.g., F1c, F2c, F3c regions) that may be used to form primers for LAMP amplification; a distinct 5′ region may include a portion (e.g., approximately half) of a target microRNA. The other half of the target microRNA may be fused to the 3′ end of the acceptor template so that when the 3′ end of the acceptor template is fused to the 5′ end of the donor template, the ligation of the two results in a full-length template (microRNA template) for amplification by LAMP.

As used herein sets or “pairs” of donor template and acceptor template typically refer to an individual donor template sequence and acceptor template sequence between which a full-length sequence complementary to an individual or particular target microRNA may be formed by combining (ligating) the donor and acceptor templates of the pair as described herein in the presence of the particular target microRNA.

A nucleotide region that is specific to a pair of donor and acceptor templates may refer to a sequence (and typically not a sequence that is identical to or complimentary to the microRNA sequence that the pair is targeting) that is different from and distinct from (e.g., having more than a few different nucleotide sequences) from other similarly-located regions of other donor, acceptor or full-length templates. For example, a 3′ end of the donor template and the 5′ end of the acceptor template may comprise one or more nucleotide sequence that is specific to the pair of donor and acceptor template and distinct from other similarly-located (at nucleotide positions relative to other donor and/or acceptor templates. In general, all of the donor templates directed to different target microRNAs may include similar or identical polynucleotide sequences, with the exception of the 5′ end including the region complimentary to a portion (e.g., half) of the different target microRNAs, and, in some variations, one or more F1c, F2c, and/or F3c regions at the 3′ end of the donor template. In general, all of the acceptor templates that are directed to different target microRNAs may include similar or identical polynucleotide sequences, with the exception of the 3′ end including the region complimentary to a portion (e.g., half) of the different target microRNAs, and, in some variations, one or more B1, B2 and/or B3 regions at the 5′ end of the donor template. In some variations, all of the donor and acceptor template pairs are identical with the exception of the target microRNA specific regions and one or more of the B1 and B2 regions at the 5′ end of the acceptor templates. In general, each pair of donor and acceptor template corresponding to a specific target microRNA may include a unique sequence that is different from any of the donor and target pair at one or more B1, B2, B3, F1c, F2c, F3d regions.

In general, a ligase as described herein may ligate ssDNA oligonucleotides splinted by a ssRNA. The term “splint ligase” may refer to an enzyme that is capable of ligating at least two ssDNA polynucleotides splinted by a complementary ssRNA polynucleotide and is capable of achieving ligation in less than 6 hours at molar concentrations of enzyme that are not absolutely required to be in molar excess compared to substrate. For example, see U.S. patent application 2014/0179539. The RNA splint ligase, single stranded polynucleotide and/or splint RNA may be immobilized on a matrix such as a reaction surface, or a magnetic bead to facilitate automated protocols and multiplexing reactions.

The term “polynucleotide” may include DNA, RNA or part DNA and part RNA. The polynucleotides when used in a ligation reaction with an RNA splint are preferably single stranded and may be partially or wholly complementary to at least a portion of the RNA splint. An example of a polynucleotide described herein is a ssDNA oligonucleotide comprising at least 8 nucleotides.

### Part I

Described herein are methods, apparatuses and compositions for detecting and analyzing small RNAs (e.g., microRNAs) that generally involve obtaining a sample containing RNA; annealing the RNA of interest in the sample to template DNA oligonucleotides complementary to the RNA of interest if the target RNA(s) are present; joining (ligating) the complementary DNA oligonucleotides to each other; and amplifying any joined (ligated) product to create multiple copies indicating the presence of the target RNA, and assaying an aspect of the amplification as an indication that the RNA of interest was present in the sample. Additional steps may be performed in addition to these steps and in some variations only a subset of these steps may be performed. Additionally, a sample may not contain an RNA of interest and an assay as described herein may be performed on such a sample to show that the RNA is not present in the sample. Such samples are generally assayed similarly as those containing an RNA of interest (although with different results) and are considered as samples or samples containing an RNA of interest as described herein.

The step of obtaining a sample containing an RNA of interest may include obtaining a synthetic RNA or a naturally occurring RNA. A synthetic RNA may be, for example, synthesized in vitro using an enzyme, a nucleic acid synthesizer, etc. A naturally occurring RNA may come from any biological sample such as a biopsy, blood, cerebrospinal fluid, fecal, pericardial fluid, plasma, pleural fluid, saliva, sputum, urine, etc. but in some particular examples is a blood sample for performing a point-of-care assay. A sample may be handled or treated such as to purify or partially purify the sample or to preserve the sample and prevent sample degradation before a subsequent step is performed (e.g., before its RNA is annealed to the oligonucleotides). For example, a blood sample may be centrifuged to enrich (separate) a sample into a blood plasma (liquid) portion and a blood cell portion, and either sample may be used for analysis as described herein.

After obtaining a patient or other sample, the sample containing the RNA of interest is generally annealed to DNA oligonucleotides that are complementary to the RNA of interest. The RNA of interest may be any RNA, but in general may be small (e.g., fewer than 500, fewer than 400, fewer than 300, fewer than 200, fewer than 100, fewer than 50, or fewer than 25 nucleotides. In some particular examples, it may be an miRNA such as those known in the art or yet to be discovered and may be 25, 24, 23, 22, 21, or 20 nucleotides in length. In some examples, it may be an miRNA such listed herein (and described in greater detail below).

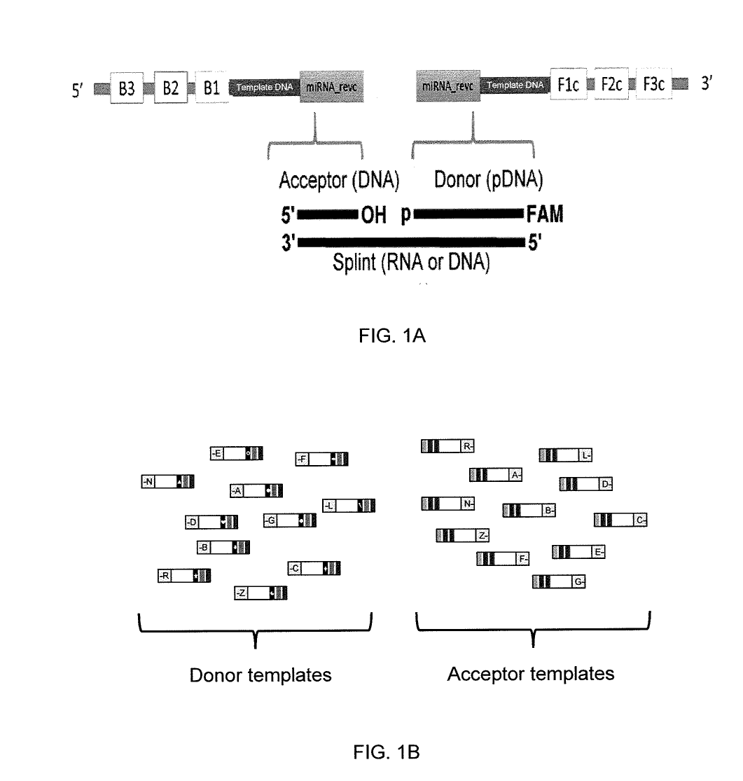

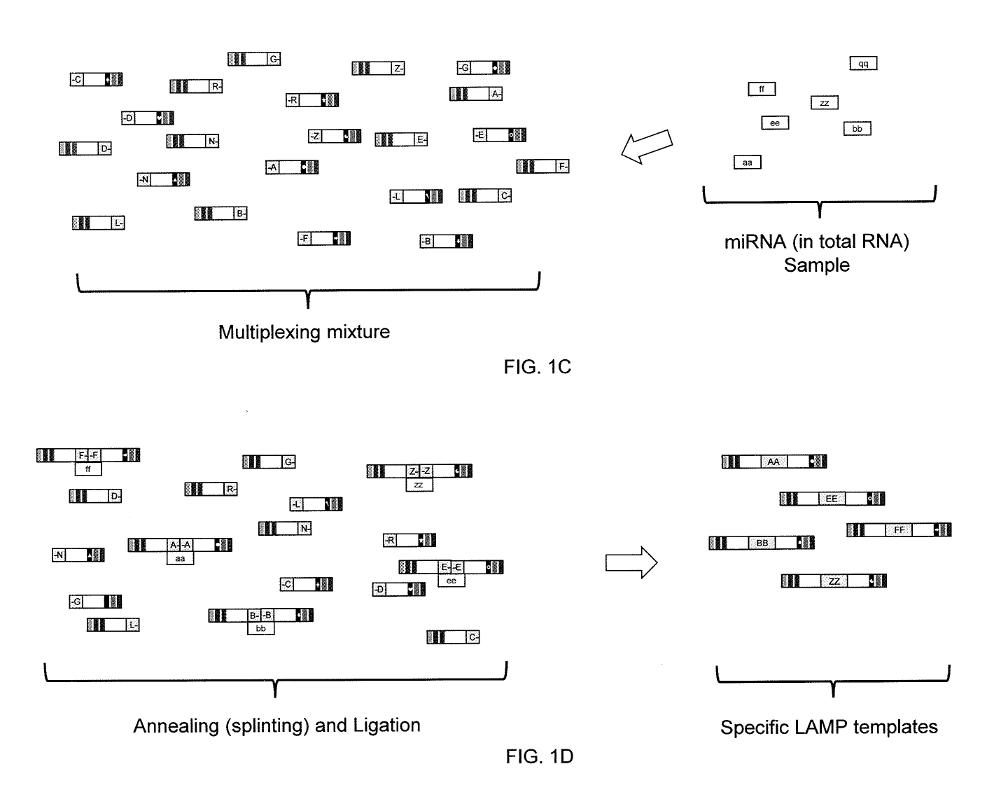

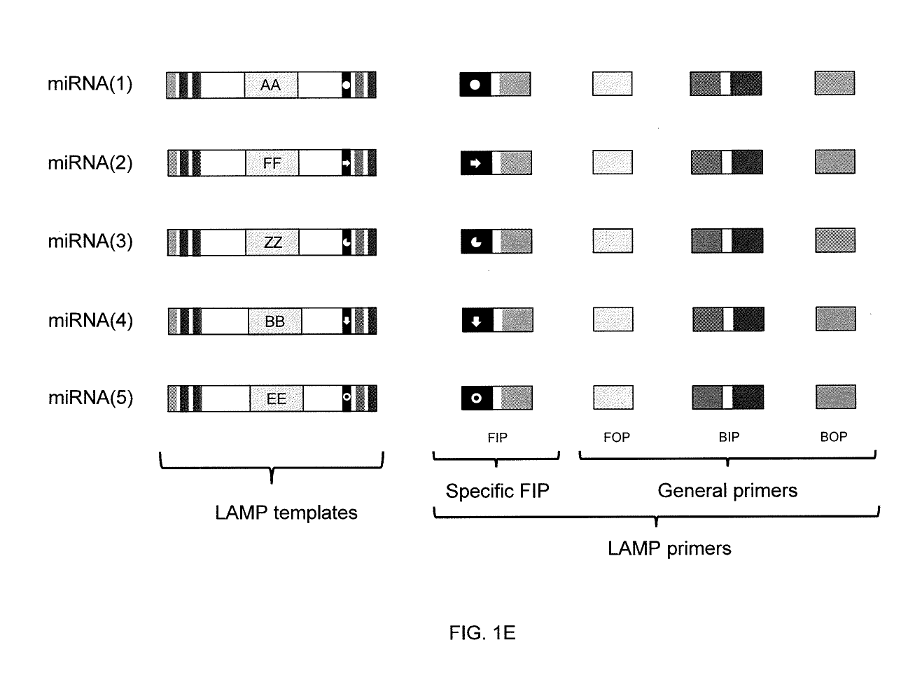

A sample containing an RNA of interest is incubated with a pair of DNA oligonucleotides, referred to herein as acceptor (DNA) and donor (pDNA) to allow the DNA oligonucleotides and RNA of interest to anneal to each other. This annealing prepares the oligonucleotides for subsequent steps by bringing them close to each other (which is utilized in the upcoming ligation step) and incorporating additional sequences (e.g., B3, B2, B1, F1c, F2c, and F3c sequence regions) into a template molecule (which is utilized in the upcoming amplification step). The target RNA may be referred to as a splint because it generally holds or splints the pair of DNA oligonucleotides close to each other, as shown in FIG. 1. Although the sample may contain many other DNA or RNA molecules, in a reaction that is highly specific, only an RNA or DNA that is complementary to both of the pair of DNA oligonucleotides will splint (hold) them together. Thus, when an RNA of interest is present the reactions described in the subsequent steps will proceed. An RNA or DNA that is not of interest, but which may nonetheless splint together the pair of oligonucleotides, will also allow the reaction to proceed (through the subsequent steps) but will create a false positive result. FIG. 1A also shows a schematic of the oligonucleotides and this part of the process. Acceptor (DNA) and donor (pDNA) each contain additional DNA sequences that will be used in later steps of the process. As indicated above, after annealing the RNA of interest in the sample to DNA oligonucleotides complementary to the RNA of interest, the next (or subsequent step) may be joining (ligating) the complementary DNA oligonucleotides to each other.

FIGS. 1B-1E schematically illustrate the first part of the methods for detecting microRNAs using a multiplexing assay, the second part of method is LAMP amplification. For example, in FIG. 1B, a plurality of pairs of acceptor and donor templates (really template “halves”) are shown schematically, with the donor templates on the right and acceptor templates on the left. For each pair of donor and acceptor template, the donor templates includes a complement of a first region of a specific target microRNA at its 5′ end, and the acceptor template include a complement of a second region from the same microRNA, where the second region is immediately adjacent to the first region. Each pair of donor and acceptor templates is specific to a particular target microRNA, and the compliment portions will hybridize to the target microRNA. In some variations the same target microRNA may be targeted by different pairs of donor and acceptor templates, for example, by choosing different region (or by differently dividing) the same microRNA.

As mentioned, in general one or more regions of either (or both) the donor and acceptor templates may include a region having a unique (with respect to the other donor and acceptor templates directed to different microRNAs) polynucleotide sequence, e.g., in a region of the 5′ end of the acceptor template and/or a 3′ end region of the donor template, such as in one or more of the B1, B2, B3, F1c, F2c, or F3c regions (schematically illustrated in FIG. 1A). The unique sequence(s) may allow for one or more LAMP primer to be generated that is specific and necessary for amplification of a particular target microRNA. In FIG. 1B, the F1c region of the donor template for each of the different pairs of templates is different, and can be used to generate specific forward inner primers (FIPs) for each pair, allowing specific detection of the microRNA associated with each template from the pooled (multiplexed) mixture. In FIG. 1B, each of the F1c regions are shown having a different symbol, indicating a different sequence. In this example, eleven different pairs of donor and acceptor are illustrated, each targeting a different microRNA, and each having a different F1c region in the donor template, although more donor and acceptor pairs may be used, targeting additional microRNAs.

The donor and acceptor pairs (in multiple copies) may be mixed together to form a multiplexing mixture, as illustrated schematically in FIG. 1C. This mixture may be liquid or solid (e.g., lyophilized) and provided as part of an apparatus or kit for performing the methods described herein. In some variations the mixture includes one or more buffers (e.g., pH buffers), salts, detergents, ATP, etc. which may be useful for maintaining the oligonucleotides of the donor and acceptor templates and/or for future ligation and/or LAMP steps, as described herein.

FIG. 1C also illustrates the addition of a patient sample including RNA (e.g., microRNAs) from which specific microRNAs will be detected. In this example, the patient sample includes copies of microRNAs (schematically illustrated in FIG. 1C). In FIGS. 1B-1E, the microRNA sequence is schematically indicated by lower-case letters (e.g., aa, bb, cc, dd, etc.), while the compliment is illustrated as upper-case letters (e.g., AA, BB, CC, DD, etc.); the adjacent portions of the microRNA sequence (complimentary sequence) that are divided up between the donor and acceptor templates are indicated by a dash (e.g., A- on the acceptor template indicates a compliment of a first region of the “aa” microRNA, and -A on the donor template indicates a compliment of a second, adjacent region, of the “aa” microRNA).

Once the sample, including microRNA has been added to the multiplexing mixture, a hybridization step (e.g., following heating to get the microRNA single stranded) may then be performed, as shown in FIG. 1D, so that target microRNA may splint to the donor and acceptor DNA templates. Thereafter, ligation using a ligase such as SplintR or T4 ligase may be performed to generate the full-length lamp templates, as shown in FIG. 1D (right side). These templates may be used along with template-specific primers, as illustrated in FIG. 1E, to amplify any of the target microRNAs from the multiplexing mixture using LAMP, as will be discussed and illustrated below. In FIG. 1E, the target microRNAs detected from the multiplexed solution by the donor and acceptor templates have formed LAMP templates. Five exemplary microRNAs were formed in this example (miRNA(1)-miRNA(5)), and for each of these a specific LAMP primer, a forward inner primer, FIP, may be generated based on each of the different F1c regions in the donor template, as schematically illustrated. The remaining three LAMP templates (forward outer primer or FOP, backwards inner primer or BIP and backwards outer primer or BOP) may be the same among all of these templates. In some variations, one or more of the other primers may be different, e.g., different FOP, BIP, and/or BOP may be used to differentially amplify other templates (e.g., corresponding to different microRNAs). As mentioned herein, in addition to the four primers described above, other primers (e.g., two additional “loop” primers may be used.

An annealed sample (e.g., as described above and herein) is generally ligated in a ligation buffer. The ligation buffer may be a buffered solution such as known in the art. However, in some variations the composition of the buffer may be optimized and may contain a salt, manganese chloride, and ATP within a range of concentrations as described herein, which may enhance the formation of the template and later amplification. In some variations the ligation buffer includes dithiothreitol (DTT). In some variations, the ligation buffer includes polyethylene glycol (PEG). In some variations such a ligation buffer lacks magnesium chloride. Use of a ligation buffer lacking magnesium chloride but containing manganese chloride reduced false positives. Manganese chloride may be present at or around a final concentration of 5 mM such as 1-5 mM, 6-10 mM or greater than 10 mM. A buffer may contain any suitable salt or combination of salts configured to control pH, such as around 7.5-7.7. For example, a buffer may contain HEPES, MES, MOPS, NaCl, Tris-HCl, Tris base, etc. In a particular example, the salt is Tris-HCl. A ligation buffer may contain ATP greater than 10 uM, around 10 uM or below 10 uM (e.g., between 1 to 10 uM, between 5 um and 10 uM, less than 10 uM, less than 9 uM, less than 8 uM, less than 7 uM, less than 6 uM, less than 5 uM or any amount in between. Use of a buffer with a relatively low level of ATP reduced false positives. In some variations, a ligation buffer may include relatively low levels of ATP, low levels of Mn++. In some variations, such a ligation buffer may further include T4 ligase.

Samples may be ligated using any DNA ligase able to ligate the oligonucleotides. For example, PBCV-1 DNA ligase (also known as Chlorella virus ligase or commercially as SplintR ligase; New England Biolabs Inc.), T4 DNA ligase, E. coli DNA ligase, etc. may be used. In some particular examples, either PBCV-1 DNA ligase (also known as Chlorella virus ligase or commercially as SplintR ligase) or T4 DNA ligase may be used. T4 DNA ligase may be used, such as around 0.5 U/l (or less than 1 U/ul, less than 5 U/ul, etc.).

In one variation, a sample may be ligated as follows. A tube containing an annealed oligonucleotides (e.g., as described herein) is placed on ice and 2.2 ul of a mixture of SplintR enzyme (NEB), T4 DNA ligase (or another ligase) in Manganese Ligation buffer 50 mM Tris-HCl, 5 mM MnCl2, 10 uM ATP, and 10 mM DTT are added. (SplintR may be added for example to a concentration of 10 nM SplintR). Samples may be incubated at 20 C to allow ligation, and the enzyme heat inactivated at 65° C. for 20 min.

In another example that has been optimized for multiplexed ligation to form multiple target microRNA templates from multiple different acceptor and donor templates, a patient sample containing total RNA is mixed with <5 nM, each of a variety of donor template and acceptor template within a single reaction vessel (e.g., tube). Thus a variety of multiple pairs of donor templates and acceptor templates (each variety directed to a specific target microRNA to be detected) may be included. The donor template and acceptor template may be DNA oligos (e.g., approximately 100 nt or less in length) and typically include adjacent regions of target miRNA sequence in their 3′ and 5′ ends, respectively. The donor DNA oligo is modified to have a phosphate group at its 5′ end. The mix of total RNA and many different miRNA (multiplexing) donor and acceptor DNA oligos may then be heated to 95° C. for 2 mins (e.g., to denature the RNA), and then steadily cooled down to room temperature for annealing to take place only when target miRNAs are present in sample. Approximately 4 nM or less of the ligase (e.g., <4 nM of SplintR or T4 DNA ligase) may be included or added together with a 1× ligation buffer containing 50 mM Tris-HCl pH 7.5, <5 uM ATP, 5 mM MnCl2, and 10 mM DTT. Ligation may then take place for approximately 30 min at 30° C., followed by inactivation of the ligase enzyme at 65° C. for 20 min. The ligation product may then serve as the template for a LAMP reaction using known methods, and described in examples below. The ligation products thus from distinct templates directed to different miRNAs and these templates differ in sequences between their B1, B2, B3, F1c, F2c, or F3c regions in a way that allows ligation to happen in this multiplexed manner while allowing specific parallel LAMP to detect only target microRNAs in specific wells of a multiwell reaction substrate to which an aliquot of the master mix (multiplex mixture) is added. The template specific (and therefore miRNA specific) LAMP primers may be arranged in a known pattern within the wells of the multiwell reaction substrate (e.g., configured as a 96 well plate), generating signal only if the template was indeed ligated thanks to target miRNA presence.

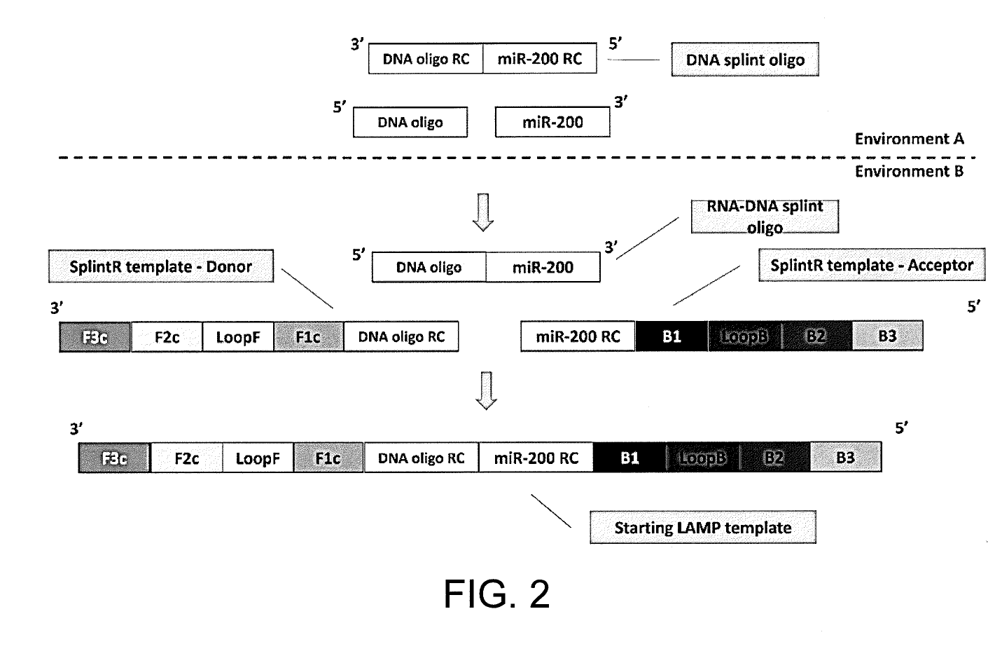

The method for multiplexed detection of microRNAs described above may be considered a two-part method. The first part is a multiplexed ligation in which partial templates for amplification by LAMP are hybridized by using an RNA “splint” including the target microRNAs to form competent templates that are amplified by the second part, involving the isothermal amplification by LAMP. FIG. 2 shows another variation of the first part of this method, in which part one is also divided up into two parts. First, a bridge oligo linked to a reverse compliment of the target microRNA sequence (e.g., having the reverse compliment of the target microRNA at the 5′ end and a DNA oligo at the 3′ end) is used as a splint for ligation of any target microRNA in a patient sample. The annealing and splinting (ligation with SplintR or T4 DNA ligase) may be accomplished as described herein. If the target microRNA is present it will therefore be annealed to the bridging DNA, and this new splint (of the target microRNA and bridging DNA) may be used as a longer (and potentially more specific and robust) splint for combining target and acceptor halves of a template pair, similar to what is described above. In this example, either the target or acceptor template halves may include at the appropriate 5′ or 3′ end the full length compliment of the target microRNA, while the other of the acceptor or target template halves is complementary to the bridging DNA. In some variations the donor or acceptor template which includes the compliment of the target microRNA may also include a specific (unique to the specific target microRNA) sequence that may be used as a LAMP primer.

For example, in FIG. 2, a bridge DNA oligo is the reverse complement of target miRNA sequence at its 5′ and of a DNA oligo at its 3′ end. Annealing of miRNA plus the DNA oligo to this bridge DNA sequence is followed by ligation of the two, producing an RNA-DNA chimeric product. This chimeric product will anneal to the donor and acceptor template halves (two DNA oligonucleotides) which incorporate additional sequences (B3, B2, B1, F3c, F2c, F1c) as described above, and the chimeric product will therefore act as a bridge to bring them together and allow their ligation into the molecule that can be amplified through LAMP. In some variations the first step (forming the DNA-RNA chimera splint) may be performed before the second part (forming the complete template). Alternatively, in some variations the first and second steps of part one are performed in the same chamber with the donor and acceptor templates, and may overlap in time.

As mentioned above, in any of these methods, after the ligation step a sample may be amplified, such as to create or increase the signal and aid in detecting the amplifying the joined (ligated) product to create multiple copies.

Amplification may be performed using loop-mediated isothermal application (LAMP) essentially as described by Tomita et al. Nat Protocol 2008; 3(5)877-82. Loop-mediated isothermal amplification (LAMP) is an amplification procedure in which the reaction can be processed at a constant temperature by one type of enzyme. The LAMP method is able to amplify a few copies of DNA to a tremendous amount in less than an hour. This technique is characterized by the use of 4-6 different primers specifically designed to recognize 6-8 distinct regions on the target gene; the reaction process proceeds at a constant temperature (e.g., 60-65° C.) and is completed within 60 min using the strand displacement reaction. Furthermore, in a LAMP assay, all steps from amplification to detection are conducted within one reaction tube under isothermal conditions.

These advantages can be used to prevent contamination, which can occur in PCR during the transfer of samples containing amplicons from tubes to gels for electrophoretic confirmation and preclude the need for complicated temperature control, as required for PCR. Therefore, the LAMP assay does not require well-equipped laboratories to be performed, and the procedure may be easily standardized among different laboratories. Unlike PCR, a denatured template is not required and DNA is generated in large amounts in a short time and positive LAMP reactions can be visualized with the naked eye. The main advantage of this technique is its simplicity; only a relatively constant temperature is needed as the amplification proceeds under isothermal conditions. The LAMP method employs a DNA polymerase and a set of four specially constructed primers that recognize six distinct sequences on the target DNA. An inner primer with sequences of sense and anti-sense strands of the target initiates LAMP. A pair of ‘outer’ primers then displaces the amplified strand with the help of a polymerase (e.g., Bst DNA polymerase) which has a high displacement activity, to release a single stranded DNA, which then forms a hairpin to initiate the starting loop for cyclic amplification. Amplification proceeds in cyclical order, each strand being displaced during elongation with the addition of new loops with every cycle. The final products are stem loop DNAs with several inverted repeats of the target and cauliflower-like structures with multiple loops due to hybridization between alternately inverted repeats in the same strand. The reaction can be accelerated by using two extra loop primers.

A set of two inner and two outer primers is required for LAMP. All four primers are used in the initial steps of the reaction, but in the later cycling steps only the inner primers are used for strand displacement synthesis. The outer primers are known as F3 (or forward outer primer, FOP) and B3 (or backward outer primer, BOP) while the inner primers are forward inner primer (FIB) and backward inner primer (BIP). Both FIP and BIP contains two distinct sequences corresponding to the sense and antisense sequences of the template DNA, one for priming in the first stage and the other for self-priming in later stages. By using an additional set of two loop primers, forward loop primer (LF) and backward loop primer (LB), the LAMP reaction time can be further reduced. The size and sequence of the primers may be chosen so that their melting temperature (Tm) is between 60-65° C., the optimal temperature for Bst polymerase. The F1c and B1c Tm values may be a little higher than those of F2 and B2 to form the looped out structure. The Tm values of the outer primers F3 and B3 have to be lower than those of F2 and B2 to assure that the inner primers start synthesis earlier than the outer primers. Additionally, the concentrations of the inner primers are higher than the concentrations of the outer primers (Notomi et al. 2000). Furthermore, it is critical for LAMP to form a stem-loop DNA from a dumb-bell structure. Various sizes of loop between F2c and F1c and between B2c and B1c have been examined and best results are given when loops of 40 nucleotides (40 nt) or longer are used. The size of template DNA may be an important factor that LAMP efficiency depends on, because the rate limiting step for amplification is strand displacement DNA synthesis. Various target sizes were tested and the best results were obtained with 130-200 by DNAs.

LAMP relies on auto-cycling strand displacement DNA synthesis which is carried out at 60-70° C. (e.g., 60-65° C.) for 45-60 min in the presence of, e.g., Bst DNA polymerase, dNTPs, specific primers and the DNA template. The mechanism of the LAMP amplification reaction includes three steps: production of starting material, cycling amplification and elongation, and recycling. To produce the starting material, inner primer FIB hybridizes to F2c in the target DNA and initiates complementary strand synthesis. Outer primer F3 hybridizes to F3c in the target and initiates strand displacement DNA synthesis, releasing a FIP-linked complementary strand, which forms a looped-out structure at one end. This single stranded DNA serves as template for BIP-initiated DNA synthesis and subsequent B3-primed strand displacement DNA synthesis leading to the production of a dumb-bell form DNA which is quickly converted to a stem-loop DNA. This then serves as the starting material for LAMP cycling, the second stage of the LAMP reaction. During cycling amplification, FIP hybridizes to the loop in the stem-loop DNA and primes strand displacement DNA synthesis, generating as an intermediate one gapped stem loop DNA with an additional inverted copy of the target sequence in the stem, and a loop formed at the opposite end via the BIP sequence. Subsequent self-primed strand displacement DNA synthesis yields one complementary structure of the original stem-loop DNA and one gap repaired stem-loop DNA with a stem elongated to twice as long and a loop at the opposite end. Both of these products then serve as templates for BIP-primed strand displacement in the subsequent cycles, the elongation and recycling step. The final product is a mixture of stem-loop DNA with various stem length and cauliflower-like structures with multiple loops formed by annealing between alternately inverted repeats of the target sequence in the same strand.

Several methods can be used to detect positive LAMP reactions, including e.g., agarose gel electrophoresis, with the gel stained by an intercalating agent such as ethidium bromide. Alternatively, given the large amount of LAMP product generated, products can be directly visualized in the reaction tube by a florescent, turbidometric or colorimetric methods. For example, after incorporation of SYBR Green I stain which has high binding affinity to DNA, product may be directly visualized. In some variations, addition of a fluorescent detection reagent (FDR) to the LAMP reaction mixture before starting the amplification allows the product to be directly visualized under UV illumination and reduces contamination. Calcein in the FDR combines initially with manganese ions and remains quenched. As pyrophosphate ions are produced as a by-product of the LAMP reaction, they bind with and remove manganese from the calcein, which results in detectable fluorescence which indicates the presence of the target genes. Alternatively, a low molecular weight PEI can be added to the LAMP product after centrifugation (e.g., for 10 s at 6000 rpm) to form an insoluble PEI-product complex containing the hybridized fluorescently labelled probe. Reaction tubes can then be visualized with a conventional UV illuminator or by fluorescence microscopy. Another method for detection of positive LAMP reactions is to monitor the increased turbidity in the reaction mixture in real-time with a turbidimeter. The turbidity is derived from precipitation of magnesium pyrophosphate generated as a by-product and this correlates with the amount of DNA amplified.

Thus, in any of the methods described herein, in general, after amplification samples can be detected using a fluorescence detector, such as a qPCR machine or convention fluorescence detector. In some variations, the detection may be qualitative and not quantitative, and may be performed by a simple technique that does not require quantification of the results, but merely determining if a resulting signal (visual, e.g., colorimetric indicator) is above positive or negative (or above a threshold), e.g., within a time period, including a predetermined time period.

In a particular example, a sample may be annealed using the techniques and oligonucleotides described herein. Briefly, samples may be annealed by combining 0.1 ul each of 1 uM acceptor and a donor DNA oligonucleotide having the specific sequences as indicated above (final concentration 5 nM), 1 ul RNA sample and water (with 0-20% additive (Betaine 1M or DMSO 10%) to a final volume of 17.8 ul were placed in microcentrifuge tubes and denatured for 2 min at 90° C. in an aluminum block in the presence of Betaine 1M or DMSO 10% or even no additive. The mix may be allowed to reach room temperature by removing the aluminum block from the heat source and allowing it to sit on the bench at room temperature until the blocks cools (e.g., for 25 min).

Samples may be ligated as follows. The tubes were placed on ice and 2.2 ul of a mixture of SplintR enzyme (NEB) in Manganese Ligation buffer 7.5-7.7 was added to a final concentration of 10 nM SplintR enzyme, 50 mM Tris-HCl, 5 mM MnCl2, 10 uM ATP, and 10 mM DTT. Samples were incubated at 20 C to allow ligation, and the enzyme heat inactivated at 65° C. for 20 min.

Samples may be amplified using Bst and related thermopile buffer (NEB). Alternatively, a 2× reaction mix containing 50 ul enzyme, 10 ul 10 mM MgSO4, 100 ul 5M betaine, 80 ul 2.5 mM dNTPS, and 10 ul water made to a total volume of 250 ul made be used. Alternatively, 1 ul of this sample may be amplified using techniques known in the art, such as, e.g., amplified using loop-mediated isothermal application (LAMP) essentially as known in the art.

In this example, levels of raw calcein fluorescence intensity from the LAMP amplification step were measured in a qPCR machine, however other quantification/detection techniques may be used. Fluorescence was plotted against sample cycle sampling; with each cycle equal to 2 minutes, and the expected fluorescence pattern obtained. Another conventional fluorescence analysis machine could be used rather than a qPCR machine for detecting the fluorescence from the calcein in the LAMP amplified sample.

### MiRNAs and Exemplary Templates

In general, the donor and acceptor templates described herein may be oligos (e.g., having 180 bp or less (e.g., 160 bp or less, 150 bp or less, 140 bp or less, 130 bp or less, 120 bp or less, 110 bp or less, 100 bp or less, 90 bp or less, etc.). As described above the sequence of the donor and acceptor regions may be optimized for the LAMP procedure and may include a portion of the target microRNA (or a compliment of the target microRNA) and may include sequence regions specifically adapted for forming the LAMP primers.

Described herein are exemplary sequences and methods and techniques for generating sequences, that may be used as discussed above. In one example, the main portion of the template (donor and acceptor and resulting whole template combining the donor and acceptor once the target microRNA sequence has been ligated as described above) is based on an intergeneic region derived from zebrafish, initially utilized to find the LAMP template. This sequence has been assessed (e.g., using commercial primer explorer software) for candidate loop primer sequences. Although initially proposed templates accommodated “space” for loop primers to bind, the sequences in-between the B1, B2 and F1, F2 regions resulted in low melting temperature loop primers (<55° C.).

Therefore, the sequence of zebrafish was modified by adding an Illumina Adapter previously tested as template in between the B1, B2 and F1, F2 regions. Following the test of the new sequence (e.g., with software, Primer explorer version 4), a set of 20 candidate loop primers pair combinations were identified. The LB, LF primer pairs did not have significant differences. Candidates with the best features were selected.

Table 1, below, provides some examples of microRNAs which are illustrated herein. This list is not exhaustive, and the same techniques for determining the microRNAs to be targeted may be applied to virtually any microRNA.

TABLE 1
exemplary miRNAs:

| Name | Sequence | SEQ ID NO |
|---|---|---|
| hsa-miR-141-3p | UAACACUGUCUGGUAAAGAUGG | SEQ ID |
| | | NO: 1 |
| hsa-miR-200b-3p | UAAUACUGCCUGGUAAUGAUGA | SEQ ID |
| | | NO: 2 |
| hsa-miR-801 | GAUUGCUCUGCGUGCGGAAUCGAC | SEQ ID |
| | | NO: 3 |
| hsa-miR-142-3p | UGUAGUGUUUCCUACUUUAUGGA | SEQ ID |
| | | NO: 4 |
| hsa-miR-451a | AAACCGUUACCAUUACUGAGUU | SEQ ID |
| | | NO: 5 |
| hsa-miR-1-3p | UGGAAUGUAAAGAAGUAUGUAU | SEQ ID |
| | | NO: 6 |
| hsa-miR-124-3p | UAAGGCACGCGGUGAAUGCC | SEQ ID |
| | | NO: 7 |
| mmu-miR-122 | UGGAGUGUGACAAUGGUGUUUG | SEQ ID |
| | | NO: 8 |
| mmu-miR-17-5p | CAAAGUGCUUACAGUGCAGGUAG | SEQ ID |
| | | NO: 9 |
| hsa-miR-16-5p | UAGCAGCACGUAAAUAUUGGCG | SEQ ID |
| | | NO: 10 |
| hsa-miR-26a-5p | UUCAAGUAAUCCAGGAUAGGCU | SEQ ID |
| | | NO: 11 |
| hsa-miR-23a-3p | AUCACAUUGCCAGGGAUUUCC | SEQ ID |
| | | NO: 12 |
| hsa-miR-210-3p | CUGUGCGUGUGACAGCGGCUGA | SEQ ID |
| | | NO: 13 |
| hsa-miR-375 | UUUGUUCGUUCGGCUCGCGUGA | SEQ ID |
| | | NO: 14 |

In order to construct the new SplintR traps, we have initially tried to examine if the new mature sequences can work with a custom template which had been proven to have the most successful design, upon appropriate modifications. The custom template had been appropriately modified to facilitate a ligation protocol (e.g., using SplintR or T4 ligase, as described above). For some of the designed LAMP templates (which may be referred to herein as “traps”), primer sequences have been modified, as indicated. In some cases some primers were designed to avoid hairpin structures with the miRNA sequence. Relevant notes have been placed below each template along with each new, miRNA specific primers.

For example, a custom template (LF,LB loops added) is illustrated in Table 2:

| label | 5′pos | 3′pos | Tm | 5′dG | 3′dG | GCᵣₐₜₑ | Sequence | SEQ ID NO. |
|---|---|---|---|---|---|---|---|---|
| F3 | 940 | 959 | 59.70 | −5.75 | −4.02 | 0.50 | GAGCACGCATACTCGCATAT | SEQ ID NO: 15 |
| B3 | 1119 | 1138 | 61.20 | −6.42 | −4.91 | 0.55 | GCGTCTGAAACCTCGATTGC | SEQ ID NO: 16 |
| FIP | | | | | | | GCGCTCCTGTCAGCTCTGA- | SEQ ID NO: 17 |
| | | | | | | | ATCACACGCACACGCG | |
| BIP | | | | | | | GCTTCACGGATCAGATACCAGC- | SEQ ID NO: 18 |
| | | | | | | | TGCAGATTTCCCGTTTGAGG | |
| F2 | 962 | 977 | 60.35 | −5.07 | −7.51 | 0.69 | ATCACACGCACACGCG | SEQ ID NO: 19 |
| F1c | 1002 | 1020 | 63.61 | −7.27 | −4.60 | 0.63 | GCGCTCCTGTCAGCTCTGA | SEQ ID NO: 20 |
| B2 | 1099 | 1118 | 60.12 | −5.66 | −4.86 | 0.50 | TGCAGATTTCCCGTTTGAGG | SEQ ID NO: 21 |
| B1c | 1057 | 1078 | 62.67 | −5.26 | −6.24 | 0.55 | GCTTCACGGATCAGATACCAGC | SEQ ID NO: 22 |

The primers in Table 2 are the general primers that may not be optimized for some miRNAs. Described below are examples of specific acceptor and donor templates for the exemplary microRNAs from table 1, above, as well as notes regarding each. The identities of the sequences below are provided. The sequences listed below correspond to single oligonucleotide. The names and ( ) around a name and region are included to identity different regions and not to indicate separate oligonucleotides.

A. hsa-miR-141-3p:

(SEQ ID NO: 1)

UAACACUGUCUGGUAAAGAUGG

An acceptor template for hsa-miR-141-3p (referred to as SEQ ID NO: 23 or hsa-miR-141-3p_revComp_Acceptor) includes LF,LB loops which have been added. For example, the sequence is: SEQ ID NO: 23:

GCGTCTGAAACCTCGATTGCTGCAGATTTCCCGTTTGAGGCGTATCGGC
TCTTCTGCTTGGB1GCTGGTATCTGATCCGTGAAGCCACGTCACCCATC
TT

In this example, the different regions (B3, B2, LoopB, B1, and portion complementary to the hsa-miR-141-3p microRNA are: B3:GCGTCTGAAACCTCGATTGC, B2:TGCAGATTTCCCGTTTGAGG, LoopB:CGTATCGGCTCTTCTGCTTGG, B1GCTGGTATCTGATCCGTGAAGC, CACGTCACCCATCTT (where the underlined portion is complimentary to the hsa-miR-141-3p). Optimal secondary structure, following salt adjustment at 50 mM, has a minimum free energy of ΔG=−4.75 kcal/mol @37° C. and ΔG=−9.38 kcal/mol @25° C. Structures of the template have been also checked @25 C.° C. but not all conformations are accepted (loops are formed at the acceptor part). Although hairpins are formed, the 3′ end of the template where the miRNA is located remain accessible in all secondary structure. 7 first nts of the miRNA are placed on the acceptor part. Note that LB loops have been changed in order to avoid hairpins with the miRNA at the 3′end. For example, a

A donor template for hsa-miR-141-3p (referred to as SEQ ID NO: 24 or hsa-miR-141-3p_revComp_Donor) also includes LF,LB loops added. SEQ ID NO: 24:

TACCAGACAGTGTTATCCAGGCGCTCCTGTCAGCTCTGATCGTATCG
GCTCTTCTGCTTCGCGTGTGCGTGTGATATATGCGAGTATGCGTGCTC

In this example, the different regions (portion complimentary to the hsa-miR-141-3p, F1c, LoopF region, F2c, F3c) are:

TACCAGACAGTGTTA

TCCAG (where the underlined portion is complimentary to the hsa-miR-141-3p), F1c: GCGCTCCTGTCAGCTCTGA, LoopF: TCGTATCGGCTCTTCTGCTT, F2c: CGCGTGTGCGTGTGAT, and F3c:ATATGCGAGTATGCGTGCTC.

Optimal secondary structure, following salt adjustment at 50 mM, has a minimum free energy of ΔG=−3.10 kcal/mol @37° C. and ΔG=−6.74 kcal/mol @25° C. Structures of the template have been also checked @25 C but not all conformations are accepted (loops are formed at the donor part). The 5′ end of the template where the miRNA is located remains accessible in all secondary structure. 15 last nts of the miRNA are placed on the donor part. Note that LF loops have been changed in order to avoid hairpins with the miRNA at the 5′end.

B. hsa-miR-200b-3p

(SEQ ID NO: 2)

(TAATACTGCCTGGTAATGATGA)

An acceptor template for hsa-miR-200b (referred to as hsa-miR-200b-3p_revComp_Acceptor) is shown in SEQ ID NO: 25:

GCGTCTGAAACCTCGATTGCTGCAGATTTCCCGTTTGAGGCGTATGC
CGTCTTCTGCTTGGGCTGGTATCTGATCCGTGAAGCCACGTCACTCAT
CATTAC

This acceptor template has regions B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, and CACGTCACTCATCATTAC. Optimal secondary structure, following salt adjustment at 50 mM, has a minimum free energy of ΔG=−3.17 kcal/mol @37° C. and ΔG=−7.21 kcal/mol @25° C. All structures are checked at both temperatures and do not form undesirable loops, hairpins. 10 first nts of the miRNA are placed on the acceptor part.

A donor template for hsa-miR-200b-3p (referred to as SEQ ID NO: 26 or hsa-miR-200b-3p_revComp_Donor) is SEQ ID NO: 26:

CAGGCAGTATTATCCAGGCGCTCCTGTCAGCTCTGATCGTATGCCGTC
TTCTGCTTCGCGTGTGCGTGTGATATATGCGAGTATGCGTGCTC

In this example, the different regions (including underlined portion complimentary to the hsa-miR-200b-3p) include:

CAGGCAGTATTA

TCCAG, F1c: GCGTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure, following salt adjustment at 50 mM, has a minimum free energy of ΔG=−3.10 kcal/mol @37° C. Optimal secondary structure, following salt adjustment at 50 mM, has a minimum free energy of ΔG=−7.10 kcal/mol @25° C. Structures of the template have been also checked @25 C but not all conformations are accepted (several loops are formed at the donor part). 12 last nts of the miRNA are placed on the donor part.

C. hsa-miR-801

(SEQ ID NO: 3)

(GAUUGCUCUGCGUGCGGAAUCGAC)

An acceptor template for hsa-miR-801 (also referred to as hsa-miR-801_rev_comp_Acceptor) is SEQ ID NO: 27:

GCGTCTGAAACCTCGATTGCTGCAGATTTCCCGTTTGAGGCGTATG
CCGTCTTCTGCTTGGGCTGGTATCTGATCCGTGAAGCCACGTCACG
TCGATTCCGCAC

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, and CACGTCACGTCGATTCCGCAC. Optimal secondary structure, following salt adjustment at 50 mM, has a minimum free energy of ΔG=−3.17 kcal/mol @37° C. and ΔG=−7.46 kcal/mol @25° C. All structures are checked at both temperatures. The folding of the acceptor trap is better at 37° C. than 25° C. where we do see a hairpin formed in the 3′ end of the template (miRNA first 13 nts)

A donor template for hsa-miR-801 (also referred to as hsa-miR-801_revComp_Donor) is SEQ ID NO: 28:

GCAGAGCAATTCCAGCTGACCGCGCTCCTGTCAGTCGTATGCCGTCTT
CTGCTTCGCGTGTGCGTGTGATATATGCGAGTATGCGTGCTC

In this example, the different regions (including underlined portion complimentary to the hsa-miR-801) include:

GCAGAGCAATT

CCAG, F1c: CTGACCGCGCTCCTGTCAG, LoopF: TCGTATGCCGTCTTCTGCTT, Fc2: CGCGTGTGCGTGTGAT, F3c: ATATGCGAGTATGCGTGCTC. Optimal secondary structure, following salt adjustment at 50 mM, has a minimum free energy of ΔG=−3.06 kcal/mol @37° C. and ΔG=−6.73 kcal/mol @25° C. Structures of the template have been also checked @25 C but not all conformations are accepted (hairpins are formed at the 5′ donor part). 11 last nts of the miRNA are placed on the donor part. Note that the loop sequence and FIP region have been changed for this template pair.

(SEQ ID NO: 4)

D. hsa-miR-142-3p (TGTAGTGTTTCCTACTTTATGGA)

An acceptor template for hsa-miR-801 (also referred to as hsa-miR-142-3p_rev_comp_Acceptor) is SEQ ID NO: 29:

GCGTCTGAAACCTCGATTGCTGCAGATTTCCCGTTTGAGGCGTATGCCGT
CTTCTGCTTGGGCTGGTATCTGATCCGTGAAGCCCAGTCCATCCATAAAG
TAG

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, and CCAGTCCA

TCCATAAAGTAG

Optimal secondary structure, following salt adjustment at 50 mM, has a minimum free energy of ΔG=−4.04 kcal/mol @37° C. and ΔG=−7.69 kcal/mol @25° C. All structures are checked at both temperatures and do not form undesirable loops, hairpins. 12 first nts of the miRNA are placed on the acceptor part.

A donor template for hsa-miR-801 (also referred to as hsa-miR-142-3p_rev_comp_Donor) is SEQ ID NO: 30:

GAAACACTACATCCAGGCGCTCCTGTCAGCTCTGATCGTATGCCGTCTTC
TGCTTCGCGTGTGCGTGTGATATATGCGAGTATGCGTGCTC

In this example, the different regions (including underlined portion complimentary to the hsa-miR-801) include:

GAAACACTACA

TCCAG, F1c: GCGCTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure, following salt adjustment at 50 mM, has a minimum free energy of ΔG=−3.10 kcal/mol @37° C. and ΔG=−7.01 kcal/mol @25° C. Structures of the template have been also checked @25 C. The folding although gets worse, the 5′ end where the miRNA is located remains accessible. 11 last nts of the miRNA are placed on the donor part.

(SEQ ID NO: 5)

E. hsa-miR-451a (AAACCGTTACCATTACTGAGTT)

An acceptor template for hsa-miR-801 (also referred to as hsa-miR-451a_rev_comp_Acceptor) is SEQ ID NO: 31:

GCGTCTGAAACCTCGATTGCTGCAGATTTCCCGTTTGAGGCGTATGCCGT
CTTCTGCTTGGGCTGGTATCTGATCCGTGAAGCCCAGTCCATGAACTCAG
TAAT

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, CCAGTCCATGAACTCAGTAAT. Optimal secondary structure, following salt adjustment at 50 mM, has a minimum free energy of ΔG=−4.04 kcal/mol @37° C. and ΔG=−7.69 kcal/mol @25° C. All structures are checked at both temperatures and do not form undesirable loops, hairpins. 11 first nts of the miRNA are placed on the acceptor part.

An exemplary donor template sequence (also referred to as hsa-miR-451a_rev_comp_Donor) is: SEQ ID NO: 32:

GGTAACGGTTTTCCAGGCGCTCCTGTCAGCTCTGATCGTATCGGCTCTTC
TGCTTCGCGTGTGCGTGTGATATATGCGAGTATGCGTGCTC

In this example, the different regions (including underlined portion complimentary to the microRNA are: GGTAACGGTTTTCCAG, F1c: GCGCTCCTGTCAGCTCTGA, Loop F: TCGTATCGGCTCTTCTGCTT, F2c: CGCGTGTGCGTGTGAT, F3c: ATATGCGAGTATGCGTGCTC. Optimal secondary structure, following salt adjustment at 50 mM, has a minimum free energy of ΔG=−3.10 kcal/mol @37° C. and ΔG=−6.94 kcal/mol @25° C. Structures of the template have been also checked @25 C. The folding although gets worse, the 5′ end where the miRNA is located remains accessible. 11 last nts of the miRNA are placed on the donor part. The sequence of the loop F has been modified in this example.

(SEQ ID NO: 11)

F. hsa-miR-26a-5p (TTCAAGTAATCCAGGATAGGCT)

An acceptor template for hsa-miR-26a-5p (also referred to as hsa-miR-26a-5p_rev_comp_Acceptor) is SEQ ID NO: 33:

GCGTCTGAAACCTCGATTGCTGCAGATTTCCCGTTTGAGGCGTATGCCGT
CTTCTGCTTGGGCTGGTATCTGATCCGTGAAGCGTATGGTAGCCTATCCT
G

The different regions of the acceptor template include: B3: GCGTCTGAAACCTCGATTGC, B2:

TGCAGATTTCCCGTTTGAGG, LoopB: CGTATGCCGTCTTCTGCTTGG, B1: GCTGGTATCTGATCCGTGAAGC, and GTATGGTAGCCTATCCTG. Optimal secondary structure, following salt adjustment at 50 mM, has a minimum free energy of ΔG=−1.4 kcal/mol @37° C. and ΔG=−4.84 kcal/mol @25° C. Structures of the template have been also checked @25 C. The folding gets worse, but the 3′ end where the miRNA is located remains accessible in almost all possible RNAs foldings. 11 first nts of the miRNA are placed on the acceptor part.

An exemplary donor template sequence (also referred to as hsa-miR-26a-5p_rev_comp_Donor) is: SEQ ID NO: 34:

GATTACTTGAATCGAGGCGCTCCTGTCAGCTCTGATCGTATGCCGTCTTC
TGCTTCGCGTGTGCGTGTGATATATGCGAGTATGCGTGCTC

In this example, the different regions (including underlined portion complimentary to the microRNA are: GATTACTTGAATCGAG, F1c: GCGCTCCTGTCAGCTCTGA, LoopF: TCGTATGCCGTCTTCTGCTT, F2c :CGCGTGTGCGTGTGAT, F3c: ATATGCGAGTATGCGTGCTC. Optimal secondary structure, following salt adjustment at 50 mM, has a minimum free energy of ΔG=−2.19 kcal/mol @37° C. and ΔG=−6.21 kcal/mol @25° C. Structures of the template have been also checked @25 C. However at that temperature the miRNA is inaccessible at the donor part. 11 last nts of the miRNA are placed on the donor part.

(SEQ ID NO: 6)

G. hsa-miR-1-3p (UGGAAUGUAAAGAAGUAUGUAU)

An acceptor template for Mir-1-3p (also referred to as Mir-1-3p_revComp_Acceptor) is SEQ ID NO: 35:

GCGTCTGAAACCTCGATTGCTGCAGATTTCCCGTTTGAGGCGTATGCCGT
CTTCTGCTTGGGCTGGTATCTGATCCGTGAAGCGTCCTCCCATACATACT
TC

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, and GTCCTCCCATACATACTTC. Optimal secondary structure has a minimum free energy of ΔG=−1.4 kcal/mol @37° C., ΔG=−4.84 kcal/mol @25° C. and salt adjustment at 50 mM. The 3′ end of the template where the miRNA revComp is located remains accessible in all secondary structures at both temperatures. 11 first nts of the miRNA are placed in the acceptor part. The underlined nts are different than the ones in the previous versions of the template for let7 which have been changed them in order to avoid hairpins in the 3′ end.

An exemplary donor template sequence (also referred to as Mir-1-3p_revComp_Donor) is: SEQ ID NO: 36:

TTTACATTCCATCGTCGCGCTCCTGTCAGCTCTGATCGTATGCCGTCTTC
TGCTTCGCGTGTGCGTGTGATATATGCGAGTATGCGTGCTC

In this example, the different regions (including underlined portion complimentary to the microRNA are: TTTACATTCCATCGTC, F1c: GCGCTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure has a minimum free energy of ΔG=−1.82 kcal/mol @37° C., ΔG=−4.77 kcal/mol @25° C. and salt adjustment at 50 mM. The 5′ end of the template where the miRNA revComp is located remains accessible in all secondary structures at both temperatures. 11 last nts of the miRNA are placed in the donor part.

(SEQ ID NO: 7)

H. hsa-miR-124-3p (UAAGGCACGCGGUGAAUGCC)

An acceptor template for Mir-124-3p (also referred to as Mir-124-3p_revComp_Acceptor) is SEQ ID NO: 37:

GCGTCTGAAACCTCGATTGCTGCAGATTTCCCGTTTGAGGCGTATGCC
GTCTTCTGCTTGGGCTGGTATCTGATCCGTCTTCGTCACCAAAGGCATT
CACCGC

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTCTTCG, and TCACCAAAGGCATTCACCGC. Optimal secondary structure has a minimum free energy of ΔG=−1.4 kcal/mol @37° C., ΔG=−4.71 kcal/mol @25° C. and salt adjustment at 50 mM. The 3′ end of the template where the miRNA revComp is located remains accessible in all secondary structures at both temperatures. 12 first nts of the miRNA are placed in the acceptor part. In this example, the B1 region has been changed compared to other templates (avoiding a hairpin structure with the reverse complement of mir-124-3p's 12 nts at 3′end); different nts are underlined. The resulting BIP has also changed.

An exemplary donor template sequence (also referred to as Mir-124-3p_revComp_Donor) is: SEQ ID NO: 38:

GTGCCTTATCCGCGCTCCTGTCAGCTCTGATCGTATGCCGTCTTCTGCT
TCGCGTGTGCGTGTGATATATGCGAGTATGCGTGCTC

In this example, the different regions (including underlined portion complimentary to the microRNA are: GTGCCTTATCC, F1c: GCGCTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure has a minimum free energy of ΔG=−1.82 kcal/mol @37° C., ΔG=−4.77 kcal/mol @25° C. and salt adjustment at 50 mM. The 5′ end of the template where the miRNA revComp is located remains accessible in all secondary structures at both temperatures. 8 last nts of the miRNA are placed in the donor part.

(SEQ ID NO: 8)

I. mmu-miR-122 (UGGAGUGUGACAAUGGUGUUUG)

An acceptor template for Mir-122 (also referred to as Mir-122_revComp_Acceptor) is SEQ ID NO: 39:

GCGTCTGAAACCTCGATTGCTGCAGATTTCCCGTTTGAGGCGTATGCCGT
CTTCTGCTTGGGCTGGTATCTGATCCGTGAAGCGTGCAAATCAAACACCA
TT

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, GTGCAAATCAAACACCATT. Optimal secondary structure has a minimum free energy of ΔG=−1.4 kcal/mol @37° C., ΔG=−4.84 kcal/mol @25° C. and salt adjustment at 50 mM. The 3′ end of the template where the miRNA revComp is located remains accessible in all secondary structures at both temperatures. 11 first nts of the miRNA are placed in the acceptor part.

An exemplary donor template sequence (also referred to as Mir-122_revComp_Donor) is: SEQ ID NO: 40:

GTCACACTCCATCCTCGCGCTCCTGTCAGCTCTGATCGTATGCCGTCTT
CTGCTTCGCGTGTGCGTGTGATATATGCGAGTATGCGTGCTC

In this example, the different regions (including underlined portion complimentary to the microRNA are: GTCACACTCCATCCTC, F1c: GCGCTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure has a minimum free energy of ΔG=−1.82 kcal/mol @37° C., ΔG=−4.77 kcal/mol @25° C. and salt adjustment at 50 mM. The 5′ end of the template where the miRNA revComp is located remains accessible in all secondary structures at both temperatures. 11 last nts of the miRNA are placed in the donor part.

(SEQ ID NO: 9)

J. mmu-miR-17-5p (CAAAGUGCUUACAGUGCAGGUAG)

An acceptor template for Mir-17-5p (also referred to as Mir-17-5p_revComp_Acceptor) is SEQ ID NO: 41:

GCGTCTGAAACCTCGATTGCTGCAGATTTCCCGTTTGAGGCGTATGCCG
TCTTCTGCTTGGCGACCATTCTGATCCGTGAAGCTATCTCCTCTACCTG
CACT

The different regions of the acceptor template include: B3: GCGTCTGAAACCTCGATTGC, B2: TGCAGATTTCCCGTTTGAGG, LoopB: CGTATGCCGTCTTCTGCTTGG, B1: CGACCATTCTGATCCGTGAAGC, TATCTCCTCTACCTGCACT. Optimal secondary structure has a minimum free energy of ΔG=−1.87 kcal/mol @37° C., ΔG=−5.5 kcal/mol @25° C. and salt adjustment at 50 mM. The 3′ end of the template where the miRNA revComp is located remains accessible in all secondary structures at both temperatures. 11 first nts of the miRNA are placed in the acceptor part. In this example the B1 region has been changed (which also avoids a hairpin structure with the reverse complement of mir-17-5p's 11 nts at 3′end). The altered nts are underlined. BIP has also been changed.

An exemplary donor template sequence (also referred to as Mir-17-5p_revComp_Donor) is: SEQ ID NO: 42:

GTAAGCACTTTGAGGTCGCGCTCCTGTCAGCTCTGATCGTATGCCGTCT
TCTGCTTCGCGTGTGCGTGTGATATATGCGAGTATGCGTGCTC

In this example, the different regions (including underlined portion complimentary to the microRNA are: GTAAGCACTTTGAGGTC, F1c: GCGCTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure has a minimum free energy of ΔG=−1.82 kcal/mol @37° C., ΔG=−5.36 kcal/mol @25° C. and salt adjustment at 50 mM. The 5′ end of the template where the miRNA revComp is located remains accessible in all secondary structures at both temperatures. 12 last nts of the miRNA are placed in the donor part.

(SEQ ID NO: 10)

K. hsa-miR-16-5p (UAGCAGCACGUAAAUAUUGGCG)

An acceptor template for Mir-16-5p (also referred to as Mir-16-5p_revComp_Acceptor) is SEQ ID NO: 43:

GCGTCTGAAACCTCGATTGCTGCAGATTTCCCGTTTGAGGCGTATGCCG
TCTTCTGCTTGGGCTGGTATCTGATCCGTGAAGCTATCTGGTCGCCAAT
ATTT

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, TATCTGGTCGCCAATATTT. Optimal secondary structure has a minimum free energy of ΔG=−1.4 kcal/mol @37° C., ΔG=−3.97 kcal/mol @25° C. and salt adjustment at 50 mM. The 3′ end of the template where the miRNA revComp is located remains accessible in all secondary structures at both temperatures. 11 first nts of the miRNA are placed in the acceptor part.

An exemplary donor template sequence (also referred to as Mir-16-5p_revComp_Donor) is: SEQ ID NO: 44:

ACGTGCTGCTATAATTCTCCTCCTGTGTCGTCTGATCGTATGCCGTCTT
CTGCTTCGCGTGTGCGTGTGATATATGCGAGTATGCGTGCTC

In this example, the different regions (including underlined portion complimentary to the microRNA are: ACGTGCTGCTATAATT, F1c: CTCCTCCTGTGTCGTCTGA, LoopF: TCGTATGCCGTCTTCTGCTT, F2c: CGCGTGTGCGTGTGAT, F3c: ATATGCGAGTATGCGTGCTC. Optimal secondary structure has a minimum free energy of ΔG=−1.82 kcal/mol @37° C., ΔG=−4.15 kcal/mol @25° C. and salt adjustment at 50 mM. The 5′ end of the template where the miRNA revComp is located remains accessible in all secondary structures at both temperatures. 11 last nts of the miRNA are placed in the donor part. The F1c region in this example has been changed (which also avoids a hairpin structure with the reverse complement of mir-16-5p's 11 nts at 5′end). The altered nts are underlined. FIP has also been changed.

(SEQ ID NO: 12)

L. hsa-miR-23a-3p (AUCACAUUGCCAGGGAUUUCC)

An acceptor template for Mir-23a-3p (also referred to as Mir-23a-3p_revComp_Acceptor) is SEQ ID NO: 45:

GCGTCTGAAACCTCGATTGCTGCAGATTTCCCGTTTGAGGCGTATGCCG
TCTTCTGCTTGGGCTGGTATCTGATCCGTGAAGCCCTGGAAATCCC

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, and CCTGGAAATCCC. Optimal secondary structure has a minimum free energy of ΔG=−3.74 kcal/mol @37° C., ΔG=−7.38 kcal/mol @25° C. and salt adjustment at 50 mM. The 3′ end of the template where the miRNA revComp is located remains accessible in all secondary structures at both temperatures. 9 first nts of the miRNA are placed in the acceptor part.

An exemplary donor template sequence (also referred to as Mir-23a-3p_revComp_Donor) is: SEQ ID NO: 46:

TGGCAATGTGATTCCTCGCGCTCCTGTCAGCTCTGATCGTATGCCGTC
TTCTGCTTCGCGTGTGCGTGTGATATATGCGAGTATGCGTGCTC

In this example, the different regions (including underlined portion complimentary to the microRNA are: TGGCAATGTGATTCCTC, F1c: GCGCTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure has a minimum free energy of ΔG=−1.82 kcal/mol @37° C., ΔG=−4.77 kcal/mol @25° C. and salt adjustment at 50 mM. The 5′ end of the template where the miRNA revComp is located remains accessible in all secondary structures at both temperatures. 12 last nts of the miRNA are placed in the donor part.

M. hsa-miR-210-3p

(SEQ ID NO: 13)

(CUGUGCGUGUGACAGCGGCUGA)

An acceptor template for Mir-210-3p (also referred to as Mir-210-3p_revComp_Acceptor) is SEQ ID NO: 47:

GCGTCTGAAACCTCGATTGCTGCAGATTTCCCGTTTGAGGCGTATGCCGT
CTTCTGCTTGGGCTGGTATCTGATCCGTGAAGCCCTTCAGCCGCT

The different regions of the acceptor template include: B3: GCGTCTGAAACCTCGATTGC, B2:

TGCAGATTTCCCGTTTGAGG, LoopB: CGTATGCCGTCTTCTGCTTGG, B1: GCTGGTATCTGATCCGTGAAGC, and CCTTCAGCCGCT. Optimal secondary structure has a minimum free energy of ΔG=−3.74 kcal/mol @37° C., ΔG=−7.38 kcal/mol @25° C. and salt adjustment at 50 mM. The 3′ end of the template where the miRNA revComp is located remains accessible in all secondary structures at both temperatures. 9 first nts of the miRNA are placed in the acceptor part.

An exemplary donor template sequence (also referred to as Mir-210-3p_revComp_Donor) is: SEQ ID NO: 48:

GTCACACGCACAGTTTATCTCCTCTACTCAGCCCTCATCGTATGCCGTCT
TCTGCTTCGCGACACGCACACATATATGCGAGTATGCGTGCTC

In this example, the different regions (including underlined portion complimentary to the microRNA are: GTCACACGCACAGTTTAT, F1c: CTCCTCTACTCAGCCCTCA, LoopF: TCGTATGCCGTCTTCTGCTT, F2c: CGCGACACGCACACAT, F3c: ATATGCGAGTATGCGTGCTC. Optimal secondary structure has a minimum free energy of ΔG=−3.7 kcal/mol @37° C., ΔG=−6.59 kcal/mol @25° C. and salt adjustment at 50 mM. The 5′ end of the template where the miRNA revComp is located remains accessible in all secondary structures at both temperatures. 13 last nts of the miRNA are placed in the donor part. In this example, F1c has been changed (which avoids a hairpin structure with the reverse complement of mir-210-3p's 13 nts at 5′end). The altered nts are underlined. F2c has also been changed, and FIP has also been changed.

N. hsa-miR-375

(SEQ ID NO: 14)

(UUUGUUCGUUCGGCUCGCGUGA)

An acceptor template for Mir-375 (also referred to as Mir-375_revComp_Acceptor) is SEQ ID NO: 49:

GCGTCTGAAACCTCGATTGCTGCAGATTTCCCGTTTGAGGCGTATGCCGT
CTTCTGCTTGGGCTGGTATCTGATCTTACTAGCTTGGTTGGTCACGCGAG
CC

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCTTACTAGC, and TTGGTTGGTCACGCGAGCC. Optimal secondary structure has a minimum free energy of ΔG=−1.54 kcal/mol @37° C., ΔG=−5.35 kcal/mol @25° C. and salt adjustment at 50 mM. The 3′ end of the template where the miRNA revComp is located remains accessible in all secondary structures at both temperatures. 11 first nts of the miRNA are placed in the acceptor part. B1 has been changed (which also avoids a hairpin structure with the reverse complement of mir-375's 11 nts at 3′end). The altered nts are underlined. Thus, BIP has been changed.

An exemplary donor template sequence (also referred to as Mir-375_revComp_Donor) is: SEQ ID NO: 50:

GAACGAACAAATCTAGGCGCTCCTGTCAGCTCTGATCGTATGCCGTCTTC
TGCTTCGCGTGTGCGTGTGATATATGCGAGTATGCGTGCTC

In this example, the different regions (including underlined portion complimentary to the microRNA are: GAACGAACAAATCTAG, F1c: GCGCTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure has a minimum free energy of ΔG=−2.28 kcal/mol @37° C., ΔG=−6.15 kcal/mol @25° C. and salt adjustment at 50 mM. The 5′ end of the template where the miRNA revComp is located remains accessible in all secondary structures at both temperatures. 11 last nts of the miRNA are placed in the donor part.

### Alternative Templates and Primers for SplintR and Other Traps:

We have utilized developed LAMP templates in order to design modified acceptor, donor templates according to the multiplexing (e.g., ligation) protocol described herein. For example, custom LAMP templates are described. The target DNA sequence can be omitted depending on the secondary structure of the acceptor, donor templates.

A second custom template has been modified to this protocol. Additional modified acceptor, donor sequences are referred to as custom template 3. The modified sequences are shown and described in Table 3: Modified LAMP protocol, SplintR ligase Step:

| label | 5′pos | 3′pos | Tm | 5′dG | 3′dG | GCrate | Sequence | SEQ ID NO: |
|---|---|---|---|---|---|---|---|---|
| F3 | 940 | 959 | 59.70 | −5.75 | −4.02 | 0.50 | GAGCACGCATACTCGCATAT | SEQ ID NO: 51 |
| B3 | 1119 | 1138 | 61.20 | −6.42 | −4.91 | 0.55 | GCGTCTGAAACCTCGATTGC | SEQ ID NO: 52 |
| FIP | | | | | | | GCGCTCCTGTCAGCTCTGA- | SEQ ID NO: 53 |
| | | | | | | | ATCACACGCACACGCG | |
| BIP | | | | | | | GCTTCACGGATCAGATACCAGC- | SEQ ID NO: 54 |
| | | | | | | | TGCAGATTTCCCGTTTGAGG | |
| F2 | 962 | 977 | 60.35 | −5.07 | −7.51 | 0.69 | ATCACACGCACACGCG | SEQ ID NO: 55 |
| F1c | 1002 | 1020 | 63.61 | −7.27 | −4.60 | 0.63 | GCGCTCCTGTCAGCTCTGA | SEQ ID NO: 56 |
| B2 | 1099 | 1118 | 60.12 | −5.66 | −4.86 | 0.50 | TGCAGATTTCCCGTTTGAGG | SEQ ID NO: 57 |
| B1c | 1057 | 1078 | 62.67 | −5.26 | −6.24 | 0.55 | GCTTCACGGATCAGATACCAGC | SEQ ID NO: 58 |

### O. Mir148b

Mir148b is another exemplary microRNA. An acceptor template for Mir148b (also referred to as Mir148b_revComp_Acceptor) is SEQ ID NO: 59:

GCGTCTGAAACCTCGATTGCTGCAGATTTCCCGTTTGAGGCGTATGCCGT
CTTCTGCTTGGGCTGGTATCTGATCCGTGAAGCCACGTCACACAAAGTTC
T

The different regions of the acceptor template include: B3: GCGTCTGAAACCTCGATTGC, B2: TGCAGATTTCCCGTTTGAGG, LoopB: CGTATGCCGTCTTCTGCTTGG, B1: GCTGGTATCTGATCCGTGAAGC, and CACGTCACACAAAGTTCT. Optimal secondary structure has a minimum free energy of ΔG=−7.78 kcal/mol @37° C. Optimal secondary structure, following salt adjustment at 50 mM, has a minimum free energy of ΔG=−3.17 kcal/mol @37° C. Although hairpins are formed, the 3′ end of the template where the miRNA is located remain accessible in all secondary structure. 10 first nts of the miRNA are placed in the acceptor part.

An exemplary donor template sequence (also referred to as Mir148b_revComp_Donor) is: SEQ ID NO: 60:

GTGATGCACTGATCCAGGCGCTCCTGTCAGCTCTGATCGTATGCCGTCTT
CTGCTTCGCGTGTGCGTGTGATATATGCGAGTATGCGTGCTC

In this example, the different regions (including underlined portion complimentary to the microRNA are: GTGATGCACTGATCCAG, F1c: GCGCTCCTGTCAGCTCTGAm LoopF: TCGTATGCCGTCTTCTGCTT, F2c: CGCGTGTGCGTGTGAT, and F3c: ATATGCGAGTATGCGTGCTC. Optimal secondary structure has a minimum free energy of ΔG=−7.16 kcal/mol @37° C. Optimal secondary structure, following salt adjustment at 50 mM, has a minimum free energy of ΔG=−3.11 kcal/mol @37° C.

### P. Let7

An acceptor template for Let7 (also referred to as Let7_revComp_Acceptor) is SEQ ID NO: 61:

GCGTCTGAAACCTCGATTGCTGCAGATTTCCCGTTTGAGGCGTATGCCGT
CTTCTGCTTGGGCTGGTATCTGATCCGTGAAGCCACGTCACAACTATACA
AC

The different regions of the acceptor template include: B3: GCGTCTGAAACCTCGATTGC, B2: TGCAGATTTCCCGTTTGAGG, LoopB: CGTATGCCGTCTTCTGCTTGG, and GCTGGTATCTGATCCGTGAAGCCACGTCACAACTATACAAC. Optimal secondary structure has a minimum free energy of ΔG=−8.00 kcal/mol @37° C. Optimal secondary structure, following salt adjustment at 50 mM, has a minimum free energy of ΔG=−3.17 kcal/mol @37° C.

An exemplary donor template sequence (also referred to as Let7_revComp_Donor) is: SEQ ID NO: 62:

CTACTACCTCATCCAGGCGCTCCTGTCAGCTCTGATCGTATGCCGTCTTC
TGCTTCGCGTGTGCGTGTGATATATGCGAGTATGCGTGCTC

In this example, the different regions (including underlined portion complimentary to the microRNA are: CTACTACCTCATCCAG, F1c: GCGCTCCTGTCAGCTCTGA, LoopF: TCGTATGCCGTCTTCTGCTT, F2c: CGCGTGTGCGTGTGAT, F3c: ATATGCGAGTATGCGTGCTC. Optimal secondary structure has a minimum free energy of ΔG=−7.25 kcal/mol @37° C. Optimal secondary structure, following salt adjustment at 50 mM, has a minimum free energy of ΔG=−3.10 kcal/mol @37° C. Although hairpins are formed, the 3′ end of acceptor and the 5′end of the donor where the miRNA parts are located remain accessible in all secondary structures. 11 first nts of the miRNA are placed in the acceptor and the rest in the donor sequence. All the secondary structures, computed with mFold, have significantly improved following salt adjustment.

Another custom template (Custom template 4) has been modified accordingly to facilitate the protocols described herein. For example, loops in critical acceptor/donor miRNA binding sites are avoided. For example, one variation of an FIP sequence (SEQ ID NO: 63) is:

ACCTAGTCGCAATGCCAGCTTTTCCATCCACAATGAGAAGGAA.

Optimal secondary structure has a minimum free energy of ΔG=1.9 kcal/mol @ 60° C.

Similarly, a variation of BIP sequence (SEQ ID NO: 64) is:

GGGTGGGTGTTGATGGGACTGTTTTTCAGAAGACTTGGTCTCTGT

Optimal secondary structure has a minimum free energy of ΔG=1.01 kcal/mol @ 60° C.

### Q. Mir148b

Various different variations of acceptor and donor templates for Mir 148b are provided for illustration. For example, an acceptor template for Mir148b (also referred to as Mir148b_revComp) is SEQ ID NO: 65: AGCATCTCCAAGTACTCCATTCAGAAGACTTGGTCTCTGTGCGTTGCTTGAGCAGTTACCAGTCCCATC AACACCCACCCGATCGGAAGAGCTCGTATGCACCTAGTCGCAATGCCAGCAAGAGCTGTGAGGTTGGC TTCCTTCTCATTGTGGATGGACAAAGTTCTGTGATGCACTGA. This template includes B3, B2, LoopB, B1, F1c, LoopF, and F2c regions. Optimal secondary structure has a minimum free energy of ΔG=0.49 kcal/mol @ 60° C.

Similarly, another variation of a whole template for Let7 microRNA (Let7_revComp) that may be used as a control is SEQ ID NO: 66: AGCATCTCCAAGTACTCCATTCAGAAGACTTGGTCTCTGTGCGTTGCTTGAGCAGTTACCAGTCCCATC AACACCCACCCGATCGGAAGAGCTCGTATGCACCTAGTCGCAATGCCAGCAAGAGCTGTGAGGTTGGC TTCCTTCTCATTGTGGATGGAACTATACAACCTACTACCTCA. This template includes B3, B2, LoopB, B1, F1c, LoopF, and F2c regions. Optimal secondary structure has a minimum free energy of ΔG=0.49 kcal/mol @ 60° C.

Another variation of a Mir 148b Acceptor template (also referred to as Mir148b_revComp_Acceptor) is SEQ ID NO: 67: AGCATCTCCAAGTACTCCATTCAGAAGACTTGGTCTCTGTGCGTTGCTTGAGCAGTTACCAGTCCCATC AACACCCACCCCCACAAAGTTCTG. Optimal secondary structure has a minimum free energy of ΔG=−2.29 kcal/mol @ 37° C.

Another variation of a Mir 148b Donor (also referred to as Mir148b_revComp_Donor) is SEQ ID NO: 68: TGATGCACTGACCACCTAGTCGCAATGCCAGCAAGAGCTGTGAGGTTGGCTTCCTTCTCATTGTGGATG GAGAGATCATTGCCAGTAGGT. Optimal secondary structure has a minimum free energy of ΔG=−2.51 kcal/mol @ 37° C.

Another variation of Mir148b that may be used (including as a positive or negative control) is a whole template, and may be referred to as (Mir148b_revComp_Whole) is SEQ ID NO: 69:

AGCATCTCCAAGTACTCCATTCAGAAGACTTGGTCTCTGTGCGTTGCTTG
AGCAGTTACCAGTCCCATCAACACCCACCCCCACAAAGTTCTGTGATGCA
CTGACCACCTAGTCGCAATGCCAGCAAGAGCTGTGAGGTTGGCTTCCTTC
TCATTGTGGATGGAGAGATCATTGCCAGTAGGT

This template variation includes B3, B2, LoopB, B1, F1c, LoopF, F2c and F1c regions. Optimal secondary structure has a minimum free energy of ΔG=0.49 kcal/mol @ 60° C.

Another variation of an acceptor template for Let7 (which may be referred to as Let7_revComp_Acceptor) is SEQ ID NO: 70: AGCATCTCCAAGTACTCCATTCAGAAGACTTGGTCTCTGTGCGTTGCTTGAGCAGTTACCAGTCCCATC AACACCCACCCCCAACTATACAAC. Optimal secondary structure has a minimum free energy of ΔG=−2.29 kcal/mol @ 37° C.

This may be paired with a donor Let7 template (referred to herein as Let7_revComp_Donor) having SEQ ID NO: 71: CTACTACCTCAGGACCTAGTCGCAATGCCAGCAAGAGCTGTGAGGTTGGCTTCCTTCTCATTGTGGATG GAGAGATCATTGCCAGTAGGT. Optimal secondary structure has a minimum free energy of ΔG=−2.51 kcal/mol @ 37° C.

The full-length Let7 template (which may be used as a control and is referred to as Let7_revComp_Whole) is SEQ ID NO: 72:

AGCATCTCCAAGTACTCCATTCAGAAGACTTGGTCTCTGTGCGTTGCTTG
AGCAGTTACCAGTCCCATCAACACCCACCCCCAACTATACAACCTACTAC
CTCAGGACCTAGTCGCAATGCCAGCAAGAGCTGTGAGGTTGGCTTCCTTC
TCATTGTGGATGGAGAGATCATTGCCAGTAGGT.

Optimal secondary structure has a minimum free energy of ΔG=0.49 kcal/mol @ 60° C.

### Examples

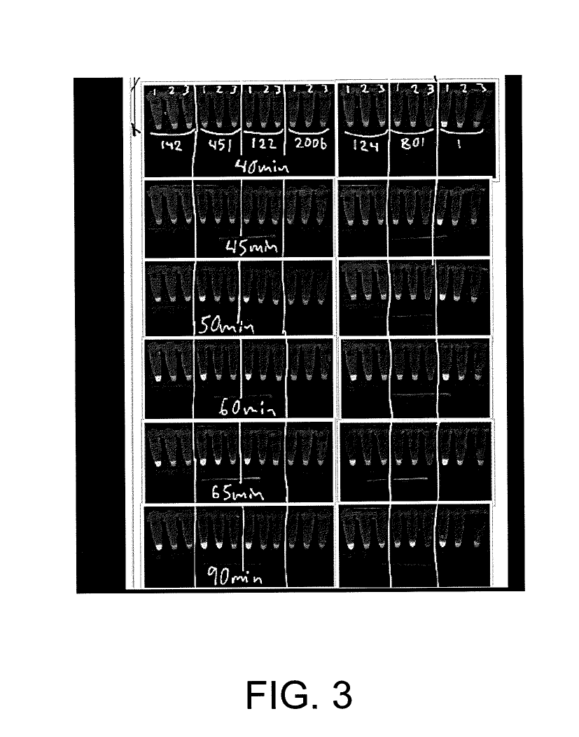

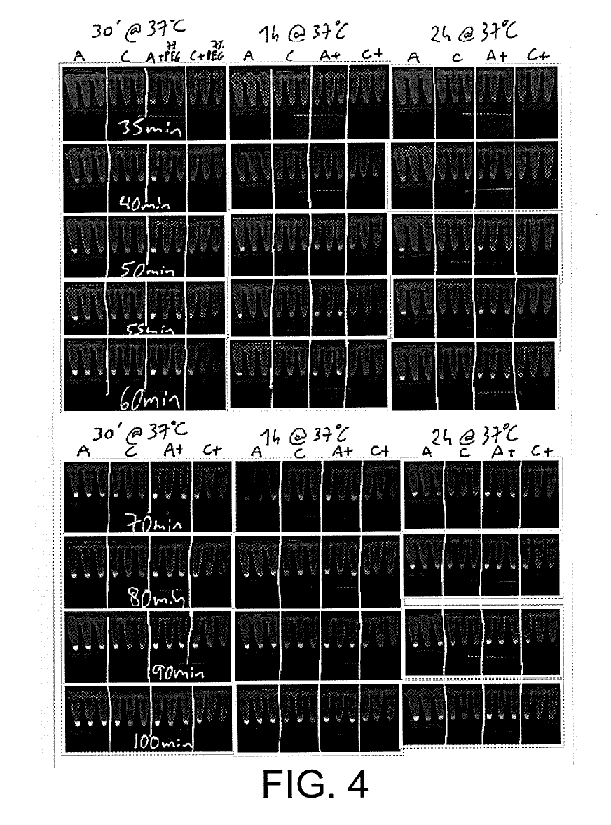

FIG. 3 shows results from performing a multiplexed assay as described herein using a high ratio of ligase (e.g., SplintR ligase) to DNA (8.4 nM ligase to 0.5 nM oligonucleotide; 16:1 ligase: DNA oligonucleotide) and assayed at 40, 45, 50, 60, 65, and 90 minutes. The assay was performed using 100 fmol spiked synthetic RNA oligonucleotides of hsa-miR142-3p, hsa-mR-451a, mmu-miR-122, hsa-miR-200b-3p, hsa-miR-124-3p, hsa-miR-801, and hsa-miR-1-3p with the sequences as described herein. Sample 2 is negative control (water). Sample 3 is a negative control with a (wrong) synthetic sequence of another miRNA oligonucleotide. Each reaction ligation reaction contained 2 ul 10× ligase buffer, 2 ul Acceptor (DNA) (5 uM), 2 ul Donor (pDNA), (5 uM), 1 ul RNA oligo (or water), and 0.16 ul SplintR ligase 1.05 uM), and 12.8 ul water. Ligations were performed at 20° C. for 30 minutes. Following ligation, LAMP reactions were performed using 6.25 ul Eiken 2× reaction mix, 0.5 ul 20 uM BIP c2, 0.5 ul 20 uM FIP c2, 0.625 10 uM B3 C2, 0.625 ul 10 uM F3c2, and 2 ul water to a total volume of 10.5 ul. Results of calcein detection are shown in FIG. 3. hsa-miR142-3p, hsa-mR-451a, mmu-miR-122, hsa-miR-124-3p, and hsa-miR-1-3p all show a strong positive signal (in tube 1) over background levels in the negative controls (tubes 2 and 3) at 60 65 minutes, and 90 minutes. In some cases, signal can be detected as early as 40 minutes (hsa-miR-1-3p).

FIG. 4 shows results from performing an assay for miR-1 as described herein using a high ratio of T4 DNA ligase, low ATP, and Mn++ without Mg++ in the ligation step. Some samples (indicated with a +) contained PEG. The assay was performed as described herein, except for the DNA ligation step which reaction was performed using the following: A): 2 ul 10 ligase buffer, 5 ul Acceptor oligo (1 uM), 5 ul Donor (p) oligo (1 uM), 1 ul RNA oligo (100 fmol), and 0.25 ul T4 DNA ligase (40 U/ul) to a final concentration of 0.5 U/ul and 6.75 ul water to a final volume of 10 ul. A+): (including PEG): 2 ul 10 ligase buffer, 5 ul Acceptor oligo (1 uM), 5 ul Donor (p) oligo (1 uM), 1 ul RNA oligo (100 fmol), and 0.25 ul T4 DNA ligase (40 U/ul) to a final concentration of 0.5 U/ul, 2.8 ul PEG 8000 50%, and 3.95 ul water to a final volume of 20 ul. C): 2 ul 10 ligase buffer, 0.1 ul Acceptor oligo (1 uM), 0.1 ul Donor (p) oligo (1 uM), 1 ul RNA oligo (100 fmol), and 0.25 ul T4 DNA ligase (40 U/ul) to a final concentration of 0.5 U/ul and 16.75 ul water to a final volume of 20 ul. C+): (including PEG): 2 ul 10 ligase buffer, 0.1 ul Acceptor oligo (1 uM), 0.1 ul Donor (p) oligo (1 uM), 1 ul RNA oligo (100 fmol), and 0.25 ul T4 DNA ligase (40 U/ul) to a final concentration of 0.5 U/ul, 2.8 ul PEG 8000 50%, and 13.75 ul water to a final volume of 20 ul. LAMP was performed using: 6.25 ul Eiken 2× reaction mix, 0.5 ul BIP c2 (20 uM), 0.5 ul FIP c2 (20 uM), 0.625 ul B3c2 (10 uM), 0.625 ul F3c2 (10 uM) and 2 ul water to a total volume of 10.5 ul. Results were assayed at 35, 40, 50, 55, 60, 70, 80, 90, and 100 minutes and are shown in FIG. 3.

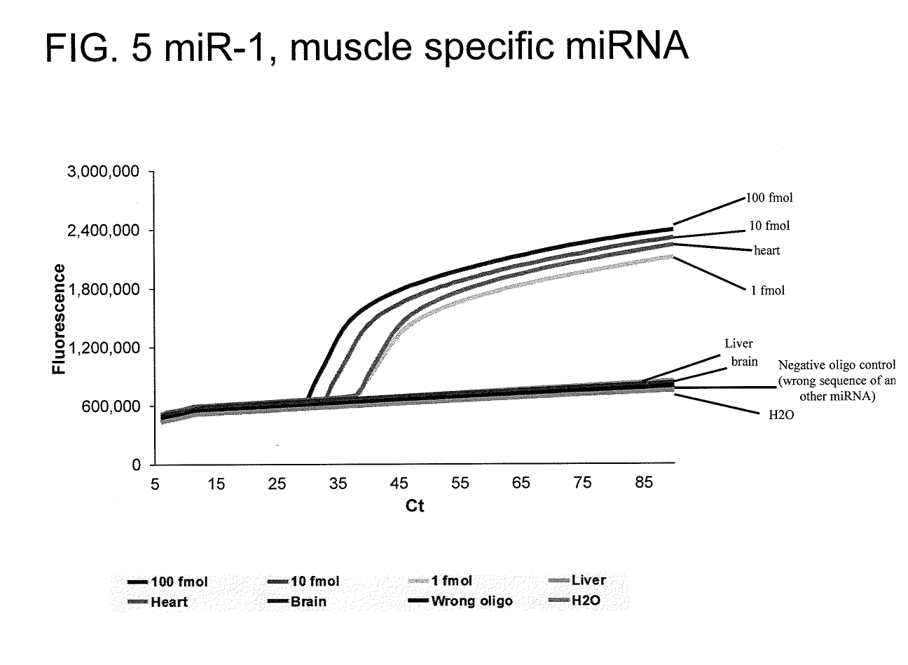

FIG. 5 shows analysis of miR-1, a muscle specific miRNA, in cardiac (heart) muscle. Samples (heart, brain, liver, and buffer control) were annealed and ligated in the presence or absence (negative controls) of miR-1 oligonucleotides using the techniques and oligonucleotides described herein. Briefly, samples were annealed by combining 0.1 ul each of 1 uM acceptor and a donor DNA oligonucleotide having the specific sequences as indicated above (final concentration 5 nM), 1 ul RNA sample with Betaine 1M and water to a final volume of 17.8 ul were placed in microcentrifuge tubes and denatured for 2 min at 90° C. in an aluminum block. The mix was allowed to reach room temperature by removing the aluminum block from the heat source and allowing it to sit on the bench at room temperature until the block cooled (e.g., for 25 min). Samples were ligated as follows. The tubes were placed on ice and 2.2 ul of a mixture of SplintR enzyme (NEB) in Manganese Ligation buffer pH 7.5-7.7 was added to a final concentration of 10 nM SplintR enzyme, 50 mM Tris-HCl, 5 mM MnCl2, 10 uM ATP, and 10 mM DTT. Samples were incubated at 20 C to allow ligation, and the enzyme heat inactivated at 65° C. for 20 min. 1 ul of this sample was amplified using loop-mediated isothermal application (LAMP) essentially as described by Tomita et al. Nat Protocol 2008; 3(5)877-82. Doi: 10.1038/nprot.2008.57. Levels of raw fluorescence intensity from the LAMP amplification step were measured in a qPCR machine. Fluorescence was plotted against sample cycle sampling; with each cycle equal to 2 minutes, and the expected fluorescence pattern obtained. Another conventional fluorescence analysis machine could be used rather than a qPCR machine for detecting the fluorescence from the calcein in the LAMP amplified sample). As predicted, miR-1 was only detected in total RNA samples from mouse cardiac muscle. Liver and brain do not express miR-1 and was not detected in these samples. Different concentrations (1 fmol, 10 fmol, and 100 fmol) of synthetic RNA oligonucleotides mimicking miR-1 presence at different levels was spiked into buffer as positive controls and detected. Negative control tests using a (wrong) synthetic sequence of another miRNA oligonucleotide and a test using no RNA input (just water) were negative.

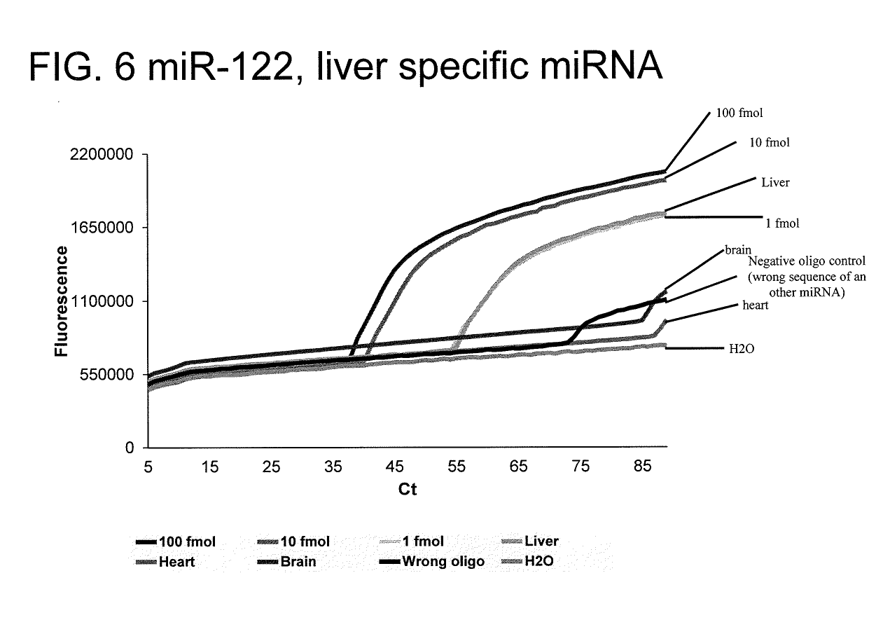

FIG. 6 shows analysis of miR-122, liver specific miRNA in liver tissue Samples (heart, brain, liver, and buffer control) were annealed and ligated in the presence or absence (negative controls) of miR-122 oligonucleotides as described for FIG. 5. As predicted, miR-1 was only detected in total RNA samples from mouse liver tissue. Heart and brain do not express miR-1 and was not detected in these samples. Different concentrations (1 fmol, 10 fmol, and 100 fmol) of synthetic RNA oligonucleotides mimicking miR-122 presence at different levels was spiked into buffer as positive controls and detected. Negative control tests using a wrong synthetic sequence of another miRNA oligonucleotide to control for non-specific effects and a test using no RNA input (just water) were negative.

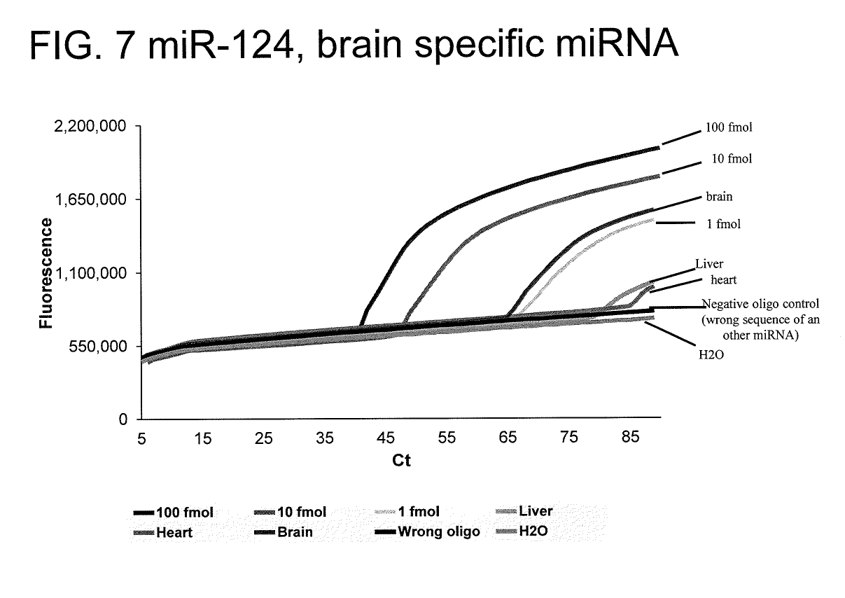

FIG. 7 shows analysis of miR-124, brain specific miRNA in brain. Samples (heart, brain, liver, and buffer control) were annealed and ligated in the presence or absence (negative controls) of miR-124 oligonucleotides as described for FIG. 5 As predicted, miR-124 was only detected in total RNA samples from mouse brain. Heart and liver do not express miR-1 and was not detected in these samples. Different concentrations (1 fmol, 10 fmol, and 100 fmol) of synthetic RNA oligonucleotides mimicking miR-124 presence at different levels was spiked into buffer as positive controls and detected. Negative control tests using a wrong synthetic sequence of another miRNA oligonucleotide to control for non-specific effects and a test using no RNA input (just water) were negative.

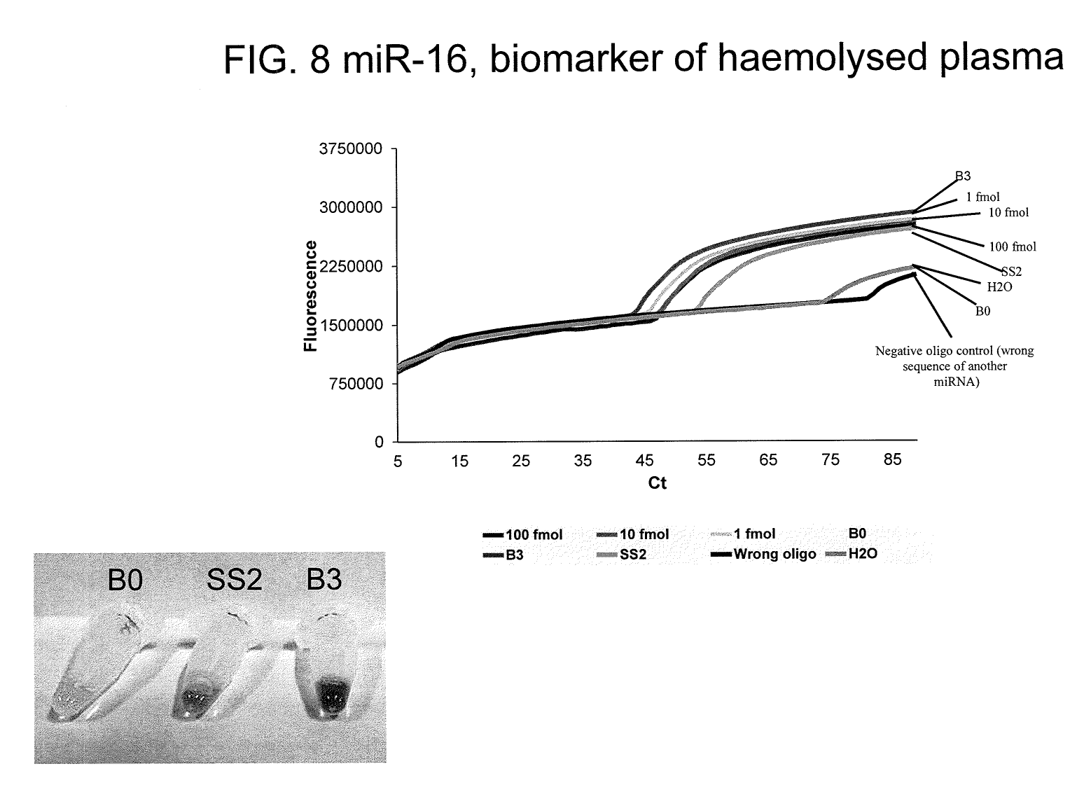

FIG. 8 shows analysis of miR-16, a biomarker of haemolysed plasma Samples (human plasma samples with and without being haemolysed and buffer control) were annealed and ligated in the presence or absence (negative controls) of miR-16 oligonucleotides as described for FIG. 5. As predicted, miR-16 was only detected in haemolysed samples. Non-hemolysed plasma samples do not express miR-16 and it was not detected in these samples. Different concentrations (1 fmol, 10 fmol, and 100 fmol) of synthetic RNA oligonucleotides mimicking miR-16 presence at different levels was spiked into buffer as positive controls and detected. Negative control tests using a wrong synthetic sequence of another miRNA oligonucleotide to control for non-specific effects and a test using no RNA input (just water) were negative. Inset shows the relatively intensities of plasma samples analyzed without (B0), a lot (B3-artificially caused) of haemolysis and with naturally haemolysed human plasma (SS2).

### Part II

In addition to the methods described above, in which the LAMP template is generated by splinted ligation using a target microRNA to allow appropriate ligation of two specifically designed template fragments, also described herein are methods and apparatuses (systems, devices, compositions, kits, etc.) in which the inner primers necessary for LAMP act as the microRNA-specific “sensor”. In this variation, one or both inner primers (traditionally referred to as FIP and BIP) are formed only in the presence of the target microRNA from two pieces (e.g., F1Cy and F2y); the sequence (or complementary sequence) of the target microRNA may form all or part of a hinge region and/or part of the template FLP/BLP recognition sequence for the LAMP template.

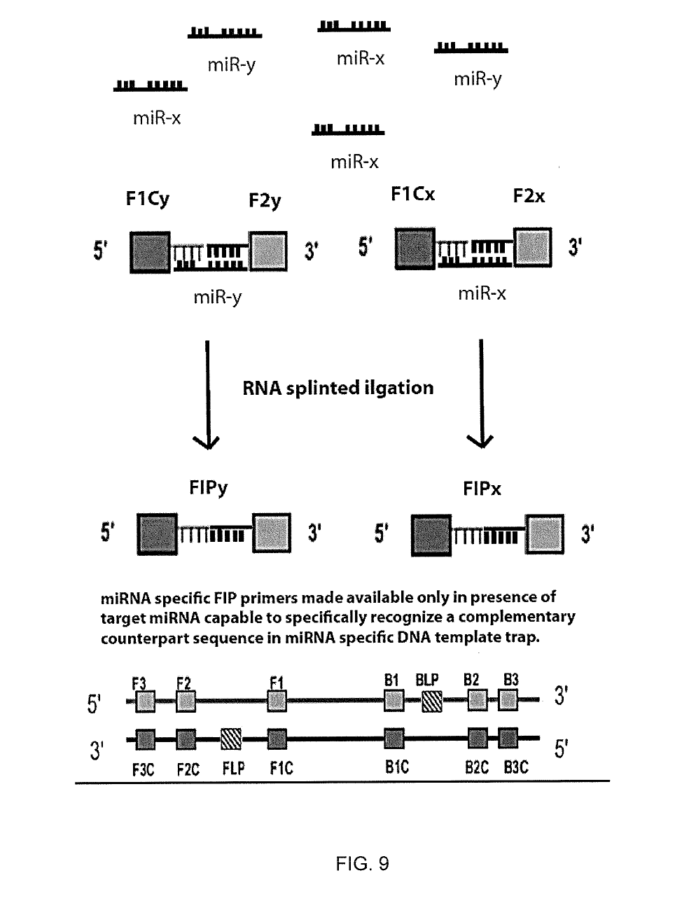

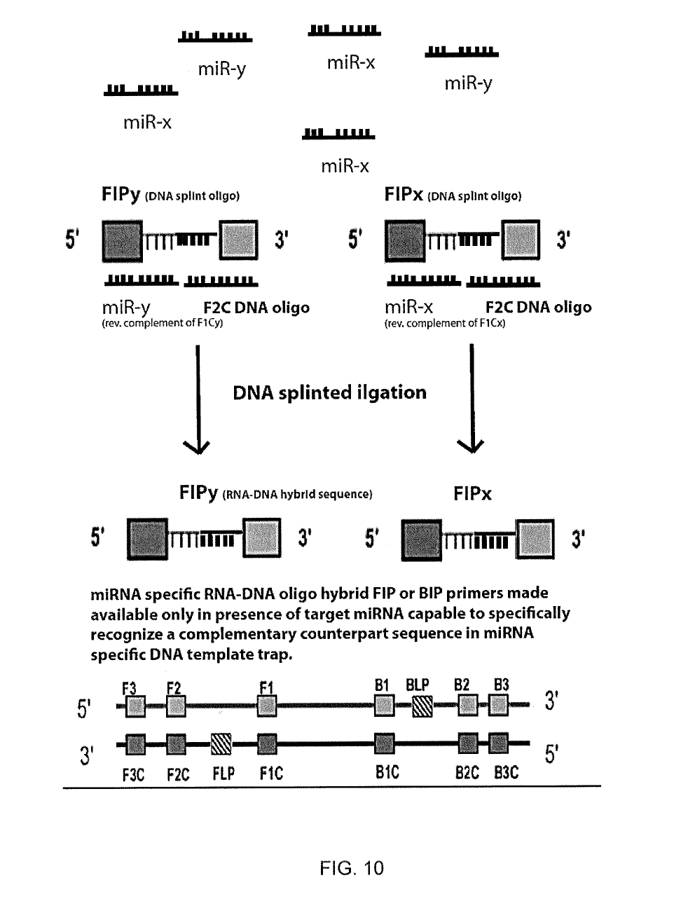

In this technique, ligatable ends (e.g., DNA oligos) are not 100 nt long, containing complementary regions for both half the miRNA and then FIP BIP and F3 and B3 primers as described in Part I, above. Instead they are very short (e.g., 20-30 nt each), complementary to part of the miRNA of interest (e.g., approximately half, divided between an A/T region) and when joined together (only in presence of the target miRNA) they produce the FIP and/or BIP (long inner primers typically >40 nt) required for LAMP. So, as opposed to the technique of part I, in which ligation mediated by the microRNA results in the LAMP template, in this variation, the template is provided with miRNA-specific FIP and/or BIP primer complementary sites. These exact FIP BIP primers will be the ligation product only in miRNA presence. Thus, in this method the LAMP (amplification) is prepared in each assay (well, chamber, region, etc.) without complete inner primers (or with only one inner primer) and thus will only result in amplification of a signal if the ligation could produce that missing inner primer (FIP or BIP). As described below (in FIGS. 9 and 10), this technique may be performed with or without an RNA splint; for example instead of an RNA splint (as shown in FIG. 9), it the target miRNA may be joined to a DNA oligo using a DNA splint.

FIG. 9 schematically illustrates one variation of this technique. In this example, the inner primer necessary for a LAMP procedure is provided only when the two partial inner primers (F1Cy and F2y) are ligated, which can only occur in the presence of the specific microRNA (e.g., miR-y in one example, or miR-x in the other example). Ligation to form the micro-RNA specific inner primer is specific to the target microRNA (e.g., miRx or miRy). In some variations only a portion of a microRNA target is needed, for example, as few as a unique 5 nt portion of the microRNA may be used.

In general, each half of the inner primer portion (e.g., F1Cy and F2y) include a portion of the target microRNA sequence (similar to what was described above). In the F1Cy example in FIG. 9, the miR-y microRNA sequence (or a portion thereof such as a 3′ region of the target miRNA, miR-y) may be complimentary to a 3′ region of the F1Cy nucleotide and an adjacent region (e.g., a 5′ region of the target miRNA, miR-y) may be complimentary to the 5′ end region of the second half of the inner primer, F2y. Thus, RNA splinted ligation (using a ligation enzyme such as splinter ligase or T4 ligase) may be used to fuse F1Cy and F2y to form FIPy. The resulting inner primer has a complementary sequence to the target microRNA (miR-y), and this region may form a part of the hinge region between the end of the inner primer. As indicated in FIG. 9, thereafter, LAMP may proceed as indicated. In this example, the F1C (e.g., F1Cy) and F2 (e.g., F2y) DNA oligos are designed such that the of 3′ of F1C and the 5′ of F2 are reverse complement in sequence to part or entire sequence of target miRNA so it can act as a splint in order to allow the ligation of F1C to F2. The ligation product constitutes a FIP specific to and capable to anneal a template (that is also miRNA specific) which can be amplified through LAMP only in the presence of this inner primer plus BIP inner primer and 2 outer primers. Both FIP and BIP can be such ligation products specific to a target miRNA.

Alternatively, the method may be performed such that DNA splinted ligation of miRNA to F2 is used. This variations is illustrated in FIG. 10. In FIG. 10, a bridge DNA oligo is the reverse complement of target miRNA sequence at its 5′ and of F2 DNA oligo at its 3′ end. Annealing of miRNA plus F2 to this bridge DNA sequence is followed by ligation whose product is the FIP primer specific to recognize and allow the amplification of a miRNA specific template through LAMP.

In both cases (RNA splinting and DNA splinting), the absence of miRNA from biological sample will mean absence of one or both inner primers for LAMP and therefore no amplification and fluorescence signal will be produced by the assay.

In use, the methods described above may be used to detect multiple microRNAs in parallel. An apparatus performing this methods may be configured to receive, for example, a tissue sample that may be prepared (e.g., homogenized, filtered, etc.) and distributed to multiple test chambers (well, regions, etc.). Test chambers may include all reagents necessary to perform the ligation (forming the inner primer as described above in part II, or forming the template as described in part I). The chamber may be thermally controlled to the proper ligation temperature(s). In some variations the same chamber(s) may also include the components necessary to perform the amplification (LAMP) procedure, including enzyme, primers, dNTPs, etc. The apparatus may then present for visual inspection the contents of the chamber, and/or may be illuminated (or visualized under ambient light) for visual inspection. In some variations an image may be taken and analyzed for the resulting product.

### Part III

As mentioned above, described herein are apparatuses that may be used to perform any of the methods described herein. In particular, described herein are apparatuses (devices, methods, kits, assays) that may be used to detect and report the presence or absence of microRNAs from a patient sample, such as a blood sample, or the like.

For example, described herein are apparatuses (systems, kits, devices, assays) that include a one or more multiwall plates (multiwall reaction substrates), which may be pre-loaded with some of the components described herein, such as the LAMP primers, enzyme, etc. (which may be lyophilized/dried, or the like), and devices (‘readers’) for controlling and reading the LAMP assay, and/or control or application software/hardware/firmware for regulating the assay and for controlling the results.

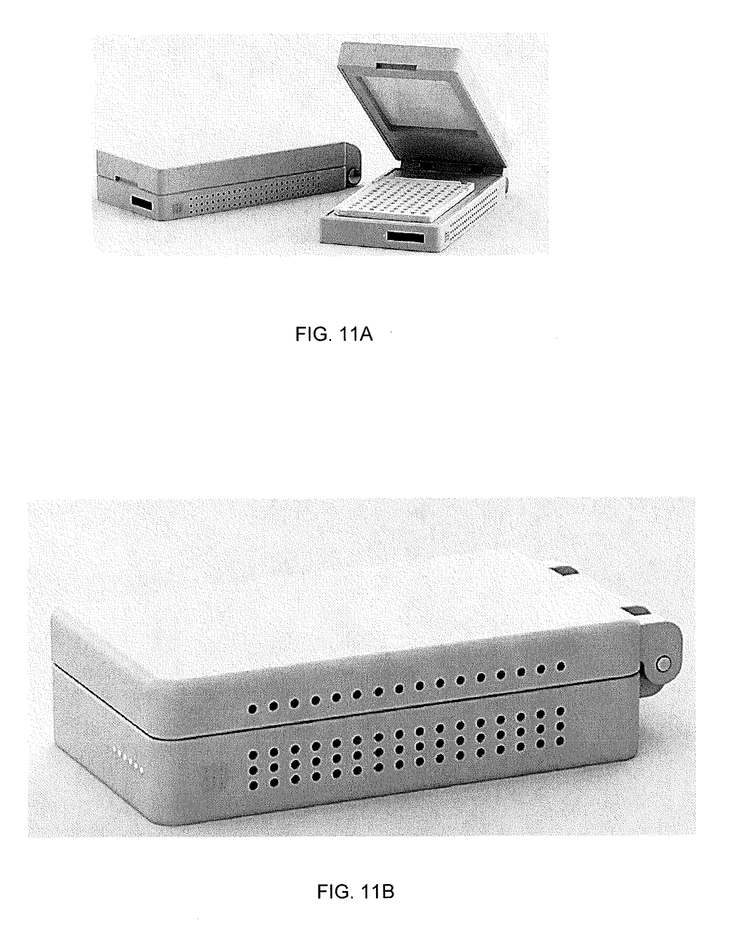

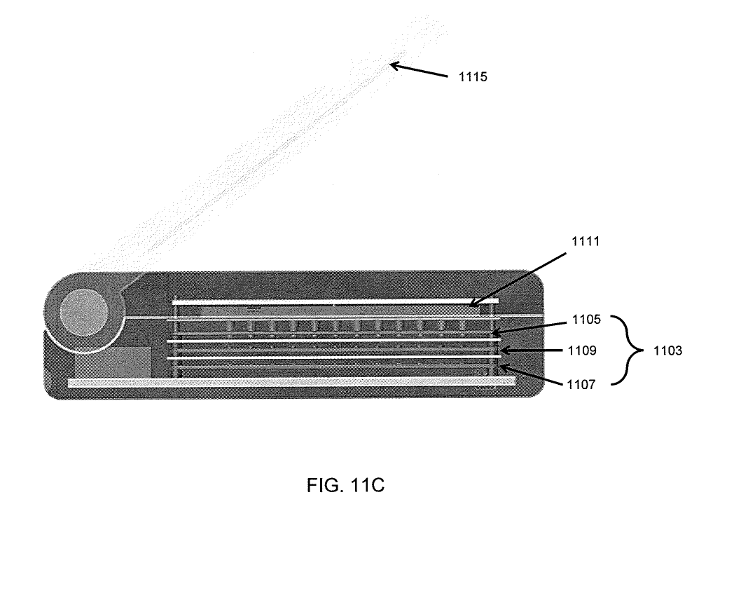

FIGS. 11A-11C illustrate one variation of a device (configured as a multiwall plate reader) configured to coordinate LAMP amplification and to detect target microRNA in any of a plurality of wells of a multiwell reaction substrate, wherein each well is associated with one specific target microRNA from the plurality of microRNAs. In general, a multiwell plate reader may include thermal control circuitry configured to maintain the temperature of each of the wells of a multiwall reaction substrate between about 60-70° C. (e.g., between about 60° C. and 65° C.) for performing the isothermal amplification (LAMP). The control circuitry may include a board having a plurality of thermal control elements configured to surround individual wells of the multiwell reaction substrate as described herein. The apparatus may also include one or more light sources configured to illuminate wells of the multiwell reaction substrate. The light source may be an LED, fiber optic (light pipe) or the like. The apparatus may also include a plurality of optical detectors, wherein each optical detector is configured to monitor a well of the multiwell reaction substrate.

Each of the apparatuses described herein is also typically configured so that it may communicate with one or more remote controllers and/or one or more remote servers. For example, in some variations the device may communicate wirelessly with a remote, handheld device such as a smartphone. Thus, in general, the apparatuses described herein may include one or more wireless communication modules that is configured to transmit sample data collected from the plurality of optical detectors to a remote processor. The wireless communication module may include Bluetooth, ultrasound, UWB, radio, or any other wireless communication technique known to be effective for the transmission of data from the patient to a remote server and (following analysis) back to the end user (e.g. patient and/or physician). For example, FIG. 11A illustrates one example of a system as described herein. In FIG. 11A, the system includes a plate reader having a housing enclosing a storage region for holding, regulating the temperature, and “reading” data from a multiwell reaction substrate (such as a 96 well dish). The device may be operated with a mobile application and may store, process and analyze each test. In FIG. 11B, the device is shown closed, having a lid that is also thermally insulated/controlled. FIG. 11C is a side view of one variation of a device such as the one described in FIGS. 11A and 11B. In FIG. 11C, the device is shown in partial section, showing the layered electronic boards 1103. In this example, the microwell reaction substrate sits within the boards, and in fact the wells may be at least partially held within holes through one of the boards, with heating elements on board, as will be described in greater detail below.

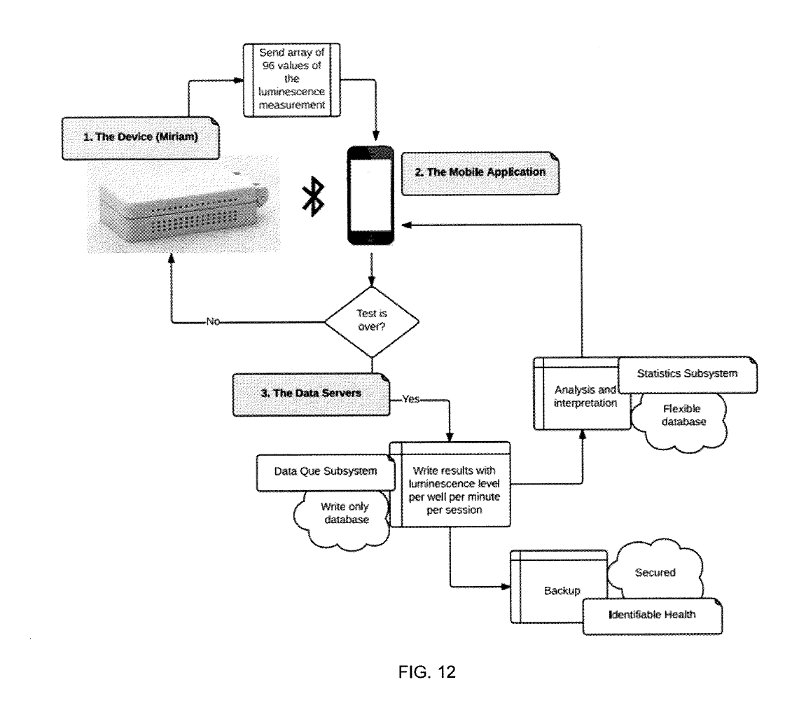

FIG. 12 illustrates a general diagram of the process, showing the operation of one example of a device (referred to as a “Miriam” device) communicating with a handheld (smartphone) device. Handheld device may be paired with the device for reliable and quick communication. The handheld device may be running an app or other control circuitry for controlling/regulating the plate reader portion of the system.

The plate reader device may be portable and lightweight (e.g., less than 10 pounds, less than 9 pounds, less than 8 pounds, less than 7 pounds, less than 6 pounds, less than 5 pounds, less than 4 pounds, less than 3 pounds, less than 2 pounds, etc.). In some variations the machine communicates with a phone wirelessly using a Bluetooth low energy protocol (BLE). In some variations a smartphone (or other handheld device) may communicate with the device by creating a BLE bridge with another chip.

For example, in some variations a laboratory technician may use the machine (reader) to: run a test cycle, store results, analyze results, and/or transmit the results (or report) to/from a physician, electronic medical record, patients, or the like. In addition, there are some tasks that the device may perform without human intervention, such as keeping a constant temperature inside chamber (e.g., between 60-70 degrees), broadcasting enqueued data to a smartphone or other hand held device, when available, writing current test status to memory (e.g., EEPROM) so the app gets alerted when a power failure happens, running hardware tests to ensure all components are healthy, and the like.

As mentioned above, the apparatus may include one or more of: a controller (e.g., a microcontroller such as the Arduino Mega1 microcontroller), circuit boards such as those described below, a wireless communications module (e.g., HC-06 Bluetooth chip), and a housing (including a lid, etc.). These components are described in greater detail herein.

The apparatus may be configured (and/or controlled by a control app/software) to sample each well with a predetermined frequency, such as once per minute, for an hour.

Thus, any of the apparatuses described herein may include or be configured to operate with, a program, software, application, or the like (hereafter “mobile application”) that may operate to control a handheld device such as a smartphone (e.g., a phone application compatible with the iOS 7+ and Android 4.4+ operating systems). The application may require a user ID and password that may be provided from an authorized third party to a patient, physician, lab, hospital, etc. The user of the mobile application may be able to: locate and list all the devices that are nearby (e.g., via Bluetooth communication); create a bridge to control a single or multiple devices; check the status of a device by ensuring all internal components pass all predefined tests; associate a plate with a patient; start a test (assay); receive luminosity results from a device for each session (e.g., at predetermined time periods such as per minute); connect to one or more servers to store the measurement results per session per minute; connect to one or more servers to receive analysis and interpretation of the results; get/sent/make an alert when the test is done; and disconnect from a device.

In addition, an app may periodically run some tasks by itself without human intervention, such as: verifying that the current session is authorized; gathering data from the Miriam machine after a test is completed; checking that a connection to an outside server can be established; saving data that is scheduled to be sent in an internal queue; sending test data to the cloud whenever an Internet connection is available.

The apparatuses described herein may generally operate with one or more remote servers. A remote server may be composed of several microservers that accept, store and process data from the mobile app clients. The servers may be configured to receive the data and may be secure to prevent unauthorized release or exposure of patient (and particularly patient-identifiable) data. The apparatus and/or servers may include a data gathering subsystem composed of one or more data gathering servers that contain a specialized application programming interface (API) that receives data from remote clients over the HTTP protocol. This subsystem may be responsible for decompressing and validating the integrity of the data sent by the client using a signature mechanism and route it to an internal data queue server. The apparatus and/or remote server(s) may also include a Data Queue Subsystem. A Data Queue Subsystem may receive data from a Data Gathering Subsystem and stores it in a processing queue waiting to be processed for another system. This sub-system may correlate data patterns with diseases, and return a list of diseases that match the data pattern. In general, the data pattern refers to the pattern of visual data received from the system (e.g., read from a plate), including data read over time.

Although there may be many ways to configure the electronics of the plate reader device to achieve the features described above, described herein (and illustrated in FIGS. 11A-22) are both general categorical descriptions as well as specific embodiments. For example, in general, the plate reader devices described herein may include a plurality of three or more printed circuit boards (both stiff and/or flexible circuit boards) that are typically arranged in a parallel arrangement (e.g., a stack) and are configured so that the plate (the multiwell reaction substrate) is held within the stack (within the arrangement) so that the wells may be directly heated from one or more of the boards, e.g., one or more boards including heating elements thereon.

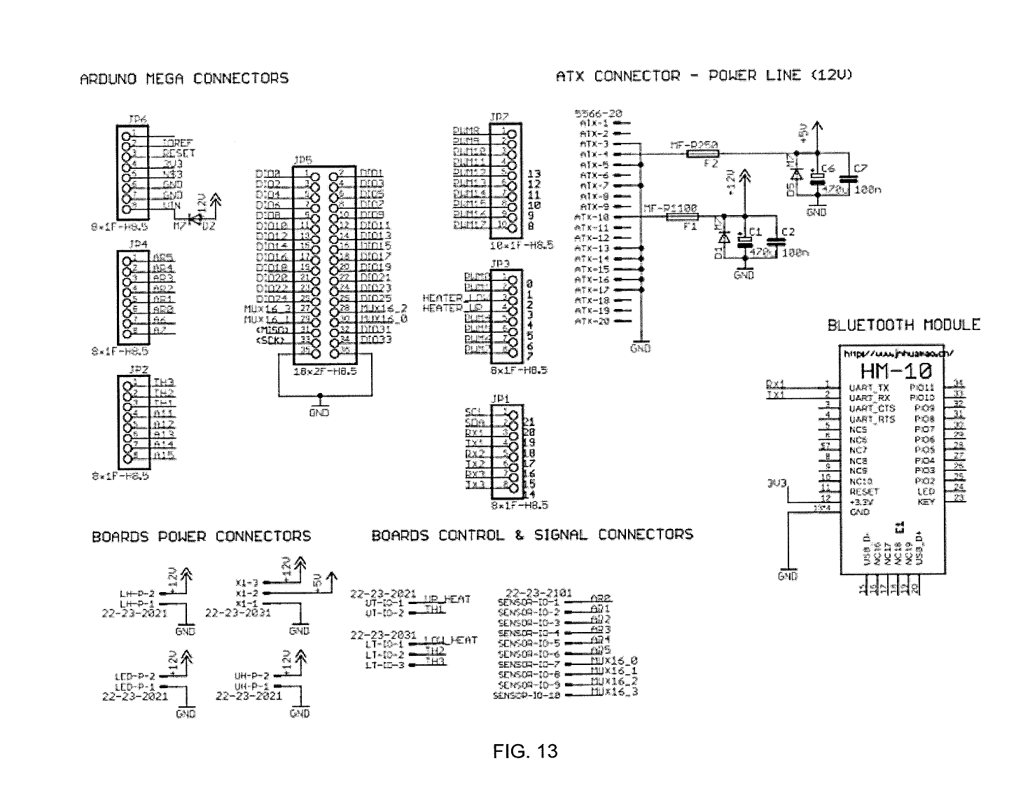

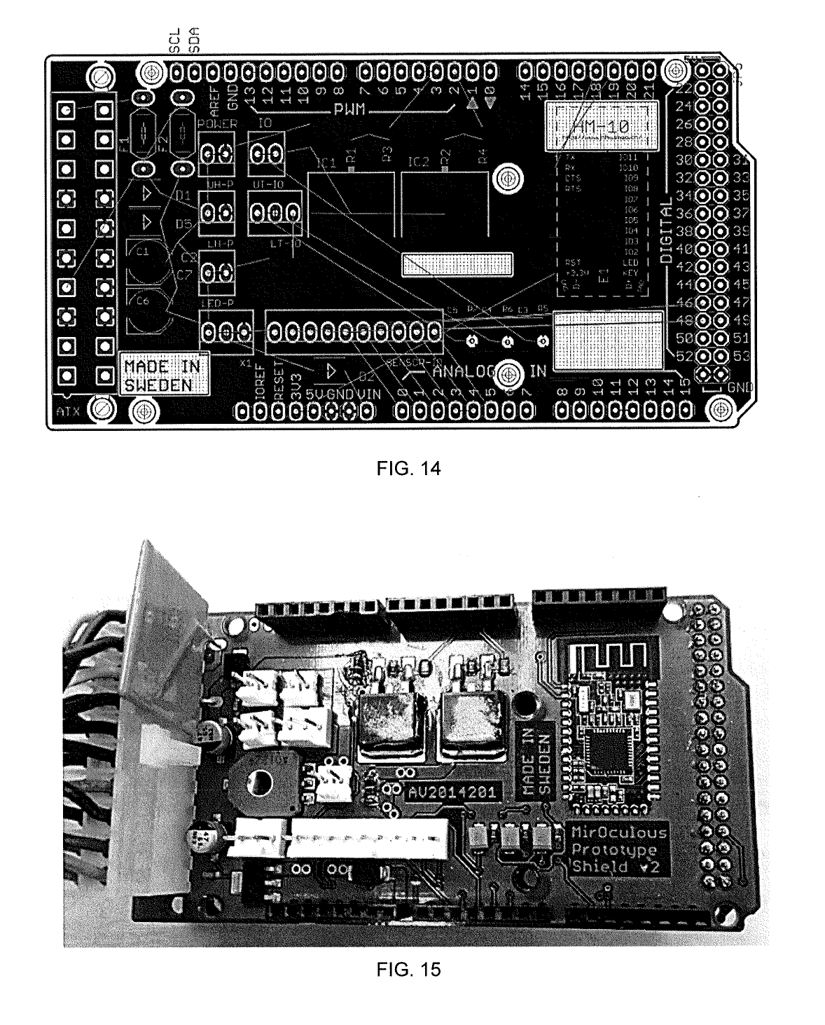

For example, as shown in FIG. 11C, one embodiment of the device includes a stack of circuit boards 1105, 1107, 1109 arranged in parallel. The multiwell reaction substrate sits in the device housing so that it projects into and through opening in one or more of the circuit boards. In FIG. 11C, the wells of the multiwell reaction substrate pass through circuit board 1105. In this example, the device includes 3 custom designed boards and 1 custom designed shield that connect to and are controlled by a processor (e.g., an Arduino microcontroller). The shield in this example integrates the custom circuit boards and the wireless communication (e.g., Bluetooth) module and integrates into the controller. For example, FIG. 13 illustrates one example of the layout of a shield including board control and signal connectors, power connectors, connection to the wireless communication module and connection to the controller. FIG. 14 is a schematic illustration showing the connections of the shield, while FIG. 15 is a picture of a prototype shield.

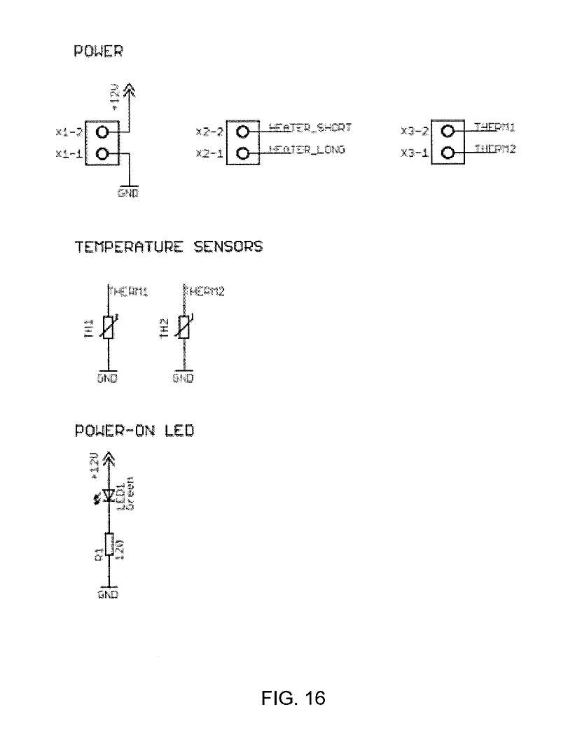

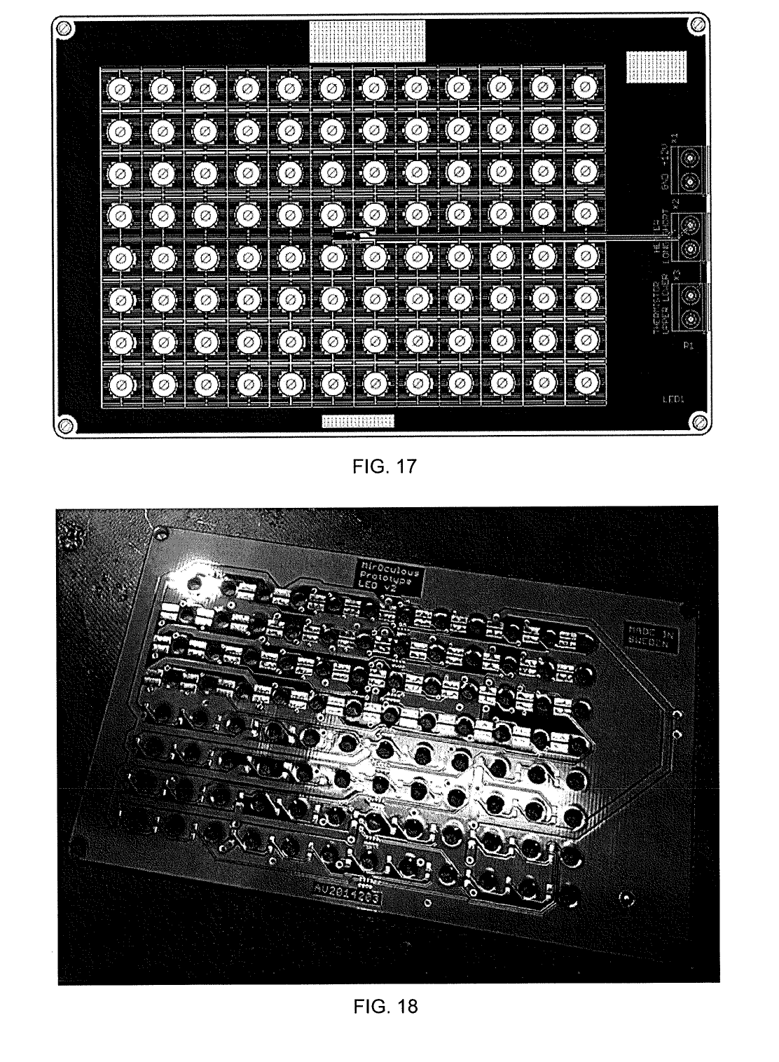

One or more of the circuit boards includes controlled heating elements (e.g., resistive heaters, thermistors, etc.). In some variations multiple circuit boards may include heating elements, for example, one in the lid/cover (“upper board” 1115) and one in the base of the device (“lower board” 1105). Either or both boards may also be referred to as temperature control boards. For example, the upper board (lid temperature control board) heats at a specific controlled temperature (within one e.g., within +/−2 degrees, 1.5 degrees, 1 degree, 0.8 degrees, 0.5 degrees, etc. of a target temp) to avoid condensation of the reaction mixture within the multiwell plate. The lower board (base temperature control board) typically heats at the specific controlled temperature (e.g., within +/−2 degrees, 1.5 degrees, 1 degree, 0.8 degrees, 0.5 degrees, etc.) and the objective is to heat the plate to regulate the temperature of the reaction (e.g., the LAMP reaction). The electronic design of the upper board and the lower board may be substantially identical. For example, FIG. 16 schematically illustrates the elements that may be present on the temperature control board(s), including power to the heating elements, temperature sensors (e.g., thermistors) providing control feedback, and one or more indicator lights (LEDs). The board(s) may also include the heating elements. FIG. 17 is a schematic showing connections for the lower board (base temperature control board). Note that the null symbol (Ø) on the figure indicates open regions, into which individual wells may be positioned. FIG. 16 shows a prototype of this board, configured to heat 96 individual wells.

The device may also include a sensor board 1109 which may hold the illuminating (light source) element(s) as well as individual detectors to detect an optical signal from each well of the multiwell plate. For example, the sensor board may consist of two arrays and a series of multiplexers for an LED array and a photodiode array. An array of LEDs, e.g., one per well on the plate, may be configured to shines light at an appropriate intensity and wavelength or wavelengths to illuminate each well. In some variations only one or a few light sources may be used, as light pipes (e.g., optical waveguides including but not limited to fiber optics, etc.) may be included to route light to each well. The controller may be configured to regulate the applied light and may illuminate the well only briefly, during imaging, or continuously. The sensor(s) may be a photodiode array. For example, the apparatus may include (e.g., on the sensor board) an array of photodiodes, one per well on the plate, that absorbs the light to identify changes in luminosity within the well. Signals collected from the sensors may be digitized (e.g., using an A/D converter that is separate from or integrated as part of the controller) and stored, processed or transmitted.

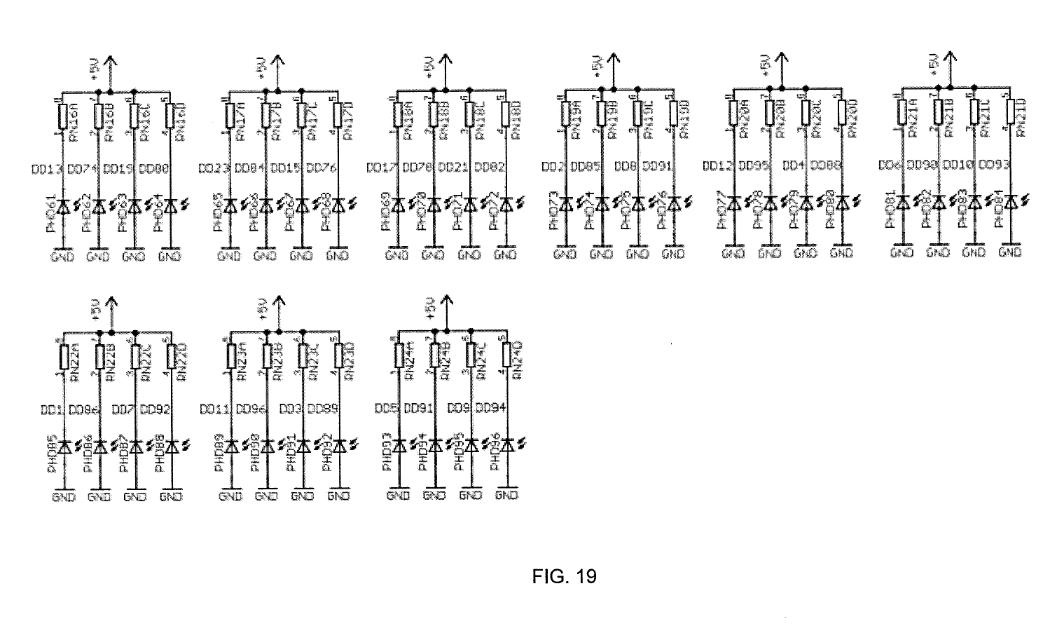

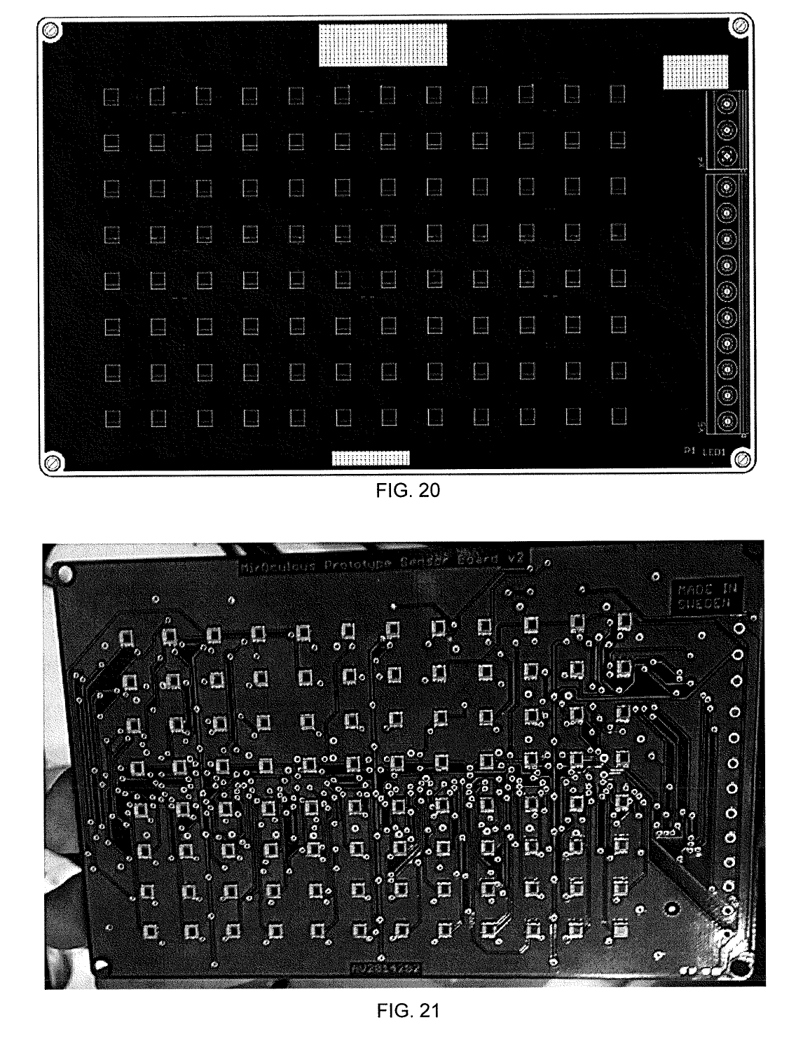

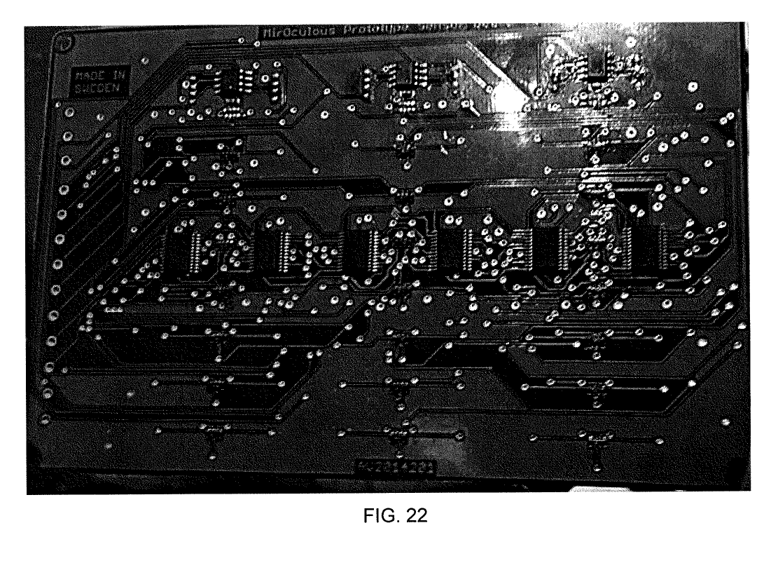

FIG. 19 shows as a schematic illustration of one variation of one variation of a layout of a sensor board, showing a partial array of LEDs. FIG. 20 is an example of a schematic of a sensor board, and FIGS. 21 and 22 shows a prototype of a sensor board including both LEDs and photodiodes (front and back).

In any of the variations described herein, the assay may be a two- or three- (or more) part assay. For example, a multiplexed assay may include an initial step using the pooled sample and the partial templates (donor and acceptor templates) to form the complete templates for amplification and detection using LAMP, as discussed above. In some variations, the initial portions (forming the full-length templates to be detected) may be performed separately from the multiwell plate and then added to the plates for parallel amplification and detection. The ligation portion (the first part(s)) may be performed in a separate reaction vessel, for example, and ligation may take place at different temperatures. Thus, in some variations the device may accommodate this by including a temperature control and/or timer(s) that are adapted for formation of complete template, as described above. For example, the device may include a separate chamber or chambers configured to control the temperature to the ranges and times useful for control of the ligation step(s), including heat inactivation. In some variations the device includes a separate heating region (chamber) into which the vessel holding the ligation mixture is heated/cooled. In some variations the same temperature control board(s) used for the LAMP portion of the assay may be used to control the temp for the ligation portion of the assay.

As mentioned, any of the apparatuses described herein may also include control logic configured as software, hardware, or firmware. For example, the plate reader devices described herein may wirelessly communicate and receive control instructions from a handheld device such as a smartphone being controlled by an application (software). This application is typically configured so that the controlling (executable) logic is stored in a non-transitory computer-readable storage medium as a set of instructions capable of being executed by a device (and particularly a handheld device such as a smartphone) to control the operation of the multiwell plate reader, and that when executed by the handheld device (e.g., smartphone), causes the smartphone to prepare for the assay, perform the assay (e.g., control the temp, sample, receive signals, etc.), process the received information and/or transmit the received information to one or more remote servers, and/or stop the assay. In some variations the application may also inform the user (doctor, patient, technician, etc.) of the results or where to receive the results of the assay.

For example, the application may be configured to operate as a mobile application program. This application may program and control the functionalities of the plate reader device through a smartphone being controlled (e.g., executing) the application. The control software may run several processes, including, for example: (INIT State) idle the apparatus controller, waiting for actions; (VERIFICATION PROCESS) initiating the assay, e.g., checking the status of the device (error checking), setting the temperature, etc., testing the temperature, testing the sensors, checking the wireless connectivity (Bluetooth test), and receiving confirmation from the device that the verification has finished; (SENSOR TEST) requesting/initiating a sensor test, performing a reading with all photodiodes (PDs), without any LEDs activated as test to give control output, putting an LED on do another PD reading and submit the data as output, receiving confirmation from the device that the test of sensors is complete, as well as any output described above; (HEAT BOARDS) requesting/initiating heating of the temperature control boards, initializing the PID for any heaters (cover temperature control board and/or base temperature control board) to the values for MIDDLE TEMP and UPPER TEMP (e.g., default values of 65° C. for the middle temperature and 90° C. for the upper temperature), receiving notification and/or passing notification to operator that the device is at temperature (and to introduce sample); (CANCEL) requesting/initiating cancelling of any assays, resetting parameters, setting temperatures IDLE_MIDDLE_TEMP and IDLE_UPPER_TEMP to the temperature control board(s), receiving confirmation that the cancellation/stop is complete, setting IDLE as a new state for the device.

The application may also set the temperature of the device. For example, the application may set the temperature for the upper temperature control board. The application may transmit a command to the device (e.g., “U 95”) to reset and update the PID. The device may respond with the value given and gives the same in the display (e.g., “UPPER BED UPDATE 95”). The application may also set the temperature of the middle or base temperature control device (e.g., send a command ‘M 65’), which may reset and update the PID. The device may respond with the value given and gives the same in the display (e.g., “MIDDLE BED UPDATE 65”).

The application may also include one or more status or help screens. For example, the application may transmit to the device a command (e.g., “h”) to print or display all the states in a one second interval. The states may be output to a serial port, and/or may be displayed sequentially.

The application may also instruct the device to display or print the temperature (e.g., temperatures of both boards in variations having two temperature control boards). For example, the application may transmit a command for printing to the device (e.g., “P”), which may send the temperature of upper board and middle board as output and display them. The device may respond with the temperature of the boards in degrees Celsius (or Fahrenheit, based on settings.

The application may control the sampling of the wells during an assay, and may otherwise coordinate the assay. For example, the apparatus may send a command to read the plates during an assay (“R”) to the device. This may put the LEDs on and read photodiodes one or multiple times (and average the multiple readings to get a single output, e.g., reading five times and calculate the average); the output from each well may then be send as output. The well values may include an indicator of their location (e.g., well number corresponding to position on the multiwell plate). The device may then indicate that the measurements are complete (e.g., “Measurement Done”).

In some variations the device and application also include a debug mode. The application may instruct the device to enter the debug mode (“D”), and the device may periodically send (e.g., every 1 s) all information about the temperature, thermoresistor values and the PWM for both boards, for debugging purposes.

In operation, the apparatus may require an operator to provide proof of identity and an indication of how or where the data (test results) are to be handled and/or presented. For example, the apparatus may be operated to require customer registration. A customer may be the operator and/or the subject/patient being tested (e.g., the source of the patient sample). For example, the system may require the customers to send proof of identity documentation and contact information, e.g., to the server. The device and/or application may be configured to require validation from the server before it can be operated to run an assay. Registration may be done automatically or aided by a support team (e.g., over a phone). The system may therefore regulate which customers and/or patients may be tested. As used herein a customer may be a patient, but is much more likely to be a clinic, hospital, laboratory, or caregiver (e.g., physician, nurse, etc.) running multiple assays of different patients. Thus, the customer may be provided with a code/identifying element that unlocks operation (or indicates the level of operation) of the device and/or app.

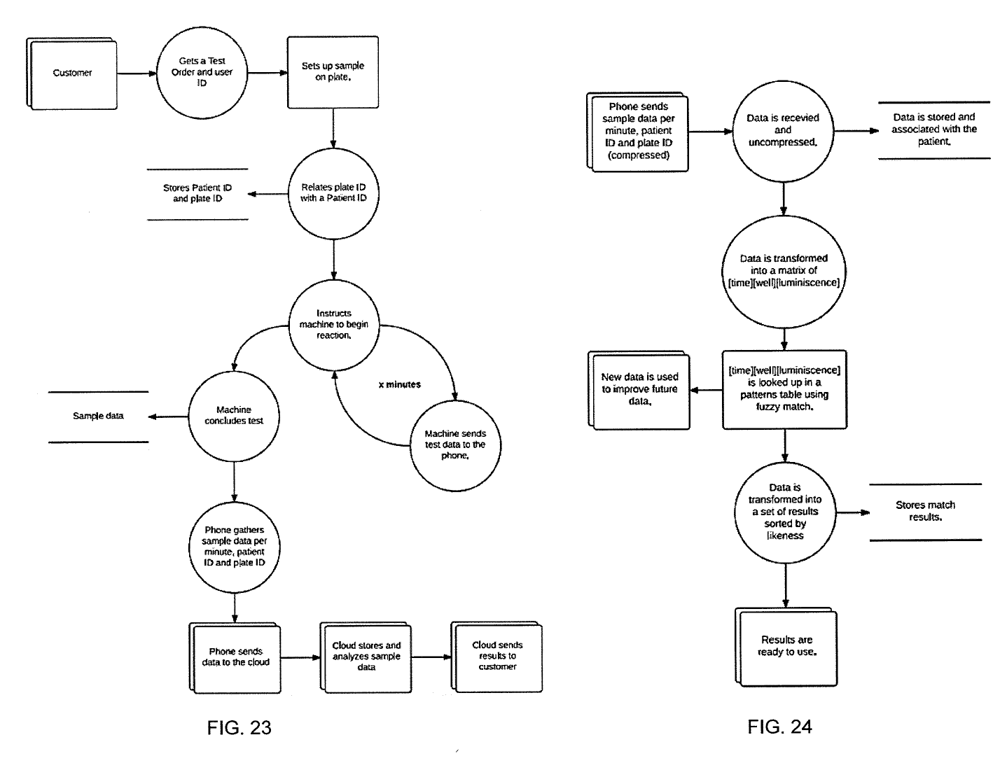

For example, FIG. 23 illustrates one example of a “test flow” for performing assays using the system described herein. In this example, the customer gets a test order and user ID (e.g., an identifying code for a particular assay), which is entered into the apparatus. Once accepted, the plate reader device can be set up to perform the assay. The assay (and particular the two- or three-part assays described herein may be performed completely with the device, or partially on the device. In this example, the data generated is sent to the controlling handheld device (e.g., smartphone, referred to as “phone”) and transmitted to a remote server (“cloud”).

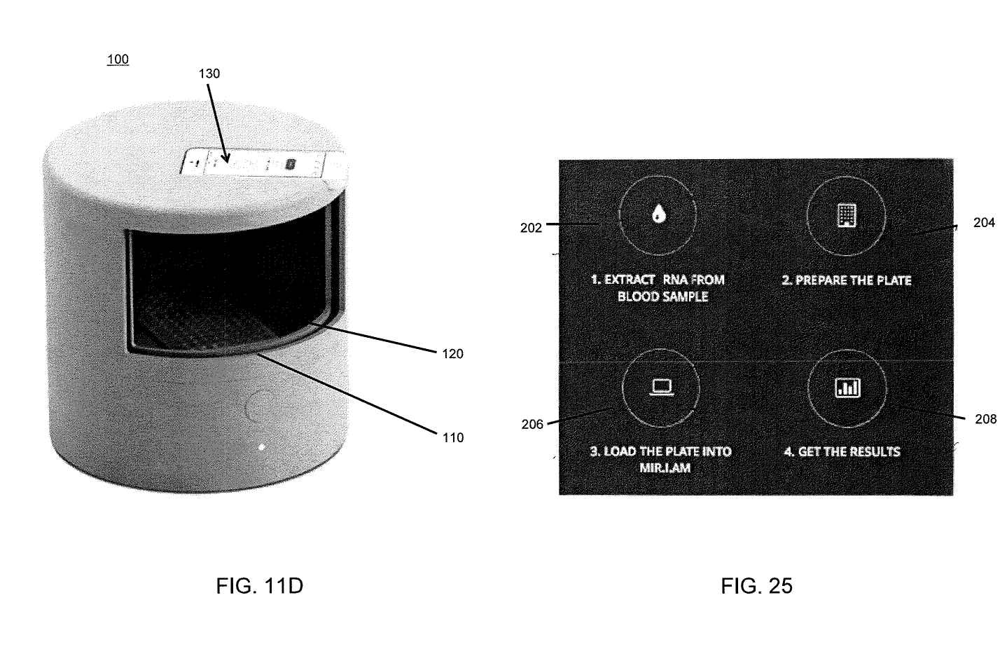

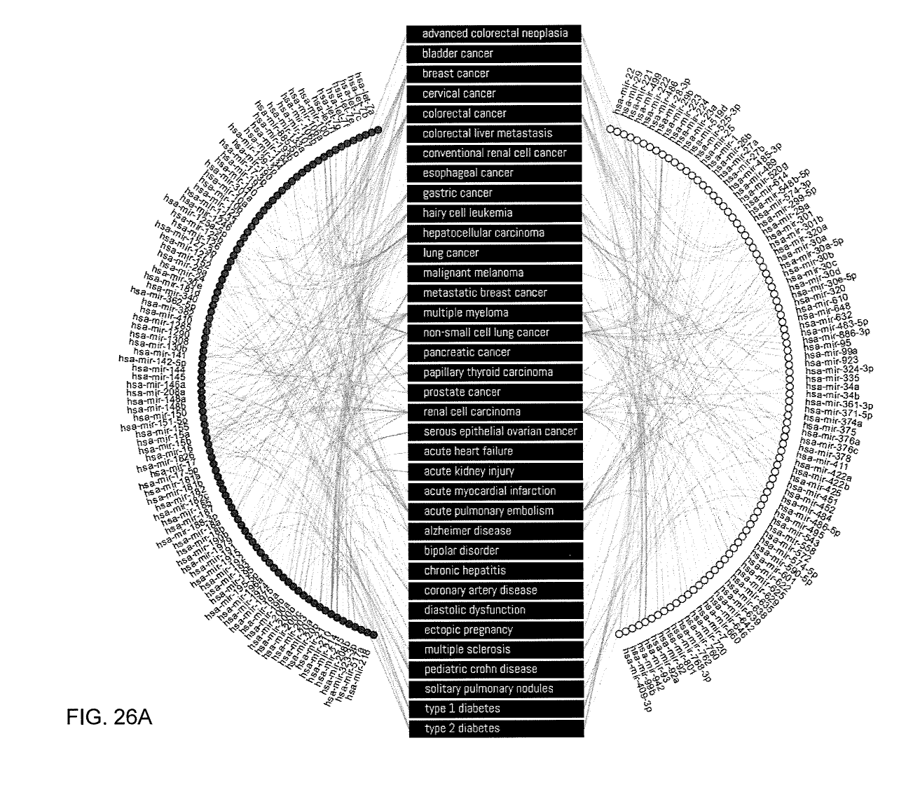

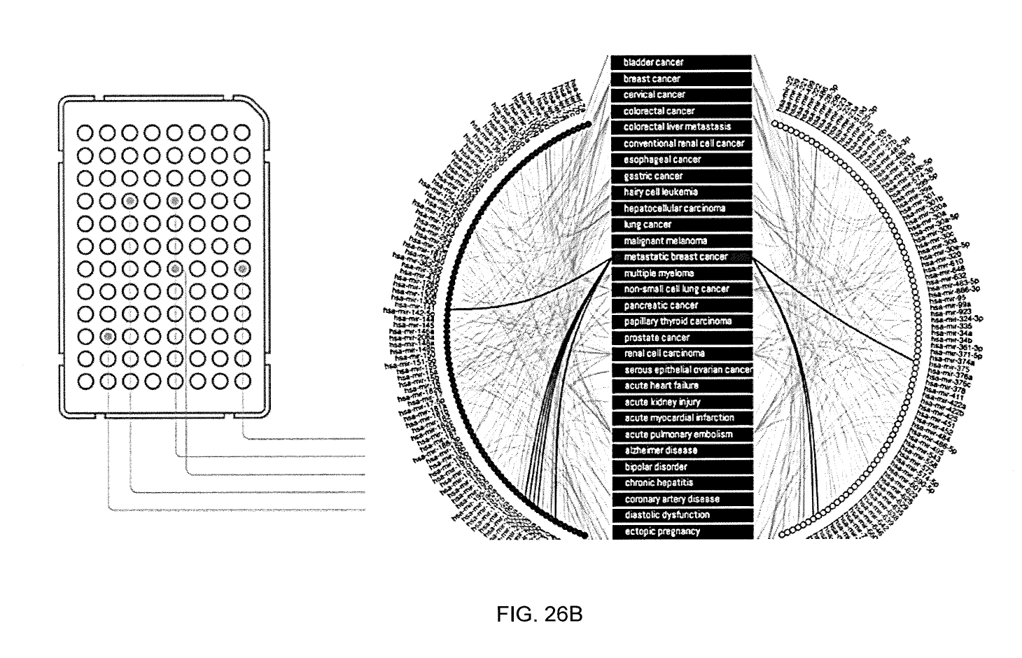

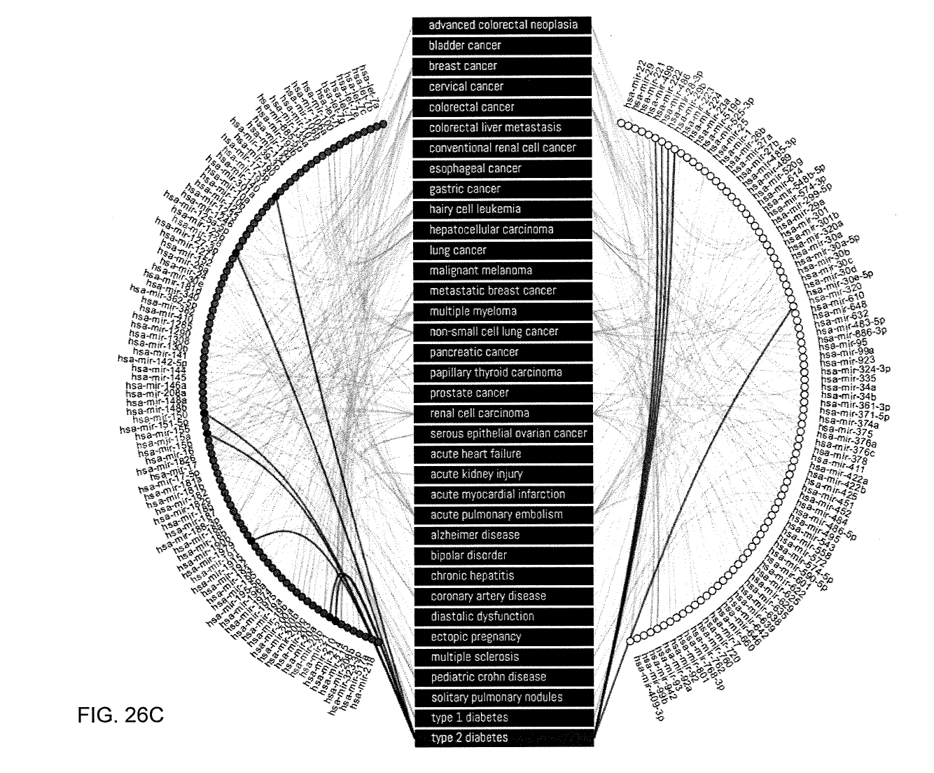

FIG. 24 schematically illustrates some of the processes that may be performed by the remote server or servers. For example, the remote server may receive the assay results and store it (e.g., in an electronic medical record associated with the patient), or may analyze it, e.g., by pattern matching to determine what the patient microRNA profile suggests about the patient health. Another example of this is provided in FIG. 26A, which illustrates multiple different microRNAs (arranged in a circular manner) and related health issue (e.g., disease states). For example, in FIG. 26A, the disease states include various cancers (prostate, renal cell carcinoma, serous epithelial ovarian cancer, papillary thyroid cancer, etc.) as well as conditions such as ectopic pregnancy, diabetes, (type 1, type 2), pediatric Crohns disease, multiple sclerosis, Alzheimer's disease, etc. These correlations are based on published microRNA links, and may be further refined by the data collected using the assays and devices described herein, as diagnoses/prognoses may be correlated with patient-specific profiles. FIG. 26B illustrates the various microRNAs modified in patient's having type 2 diabetes (indicated by connections to the listed microRNAs that may be examined as described herein). The microRNAs that may be tested, as well as the associations with the various disease states shown are not exhaustive, but merely illustrative. Additional or different microRNAs may be included and/or correlated with these (or additional) diseases and disorders.

The apparatuses described herein can generate results to the user in near real-time while creating a database of miRNAs identified in plasma that may be used to identify correlations and causations of several diseases. By thinking about disease as complex networks, system breakdowns may be identified within the body and related to specific molecules, thus drawing novel medical correlations. The methods and apparatuses described herein describe and enable the collection of data that may highlight previously unnoticed network nodes.

As discussed above, FIGS. 26A-26B illustrate a list of diseases (not limited to cancers) and unique combination of microRNAs associated with these. This information was identified both empirically and from published information. These schematic demonstrations illustrate how diseases link to each other through shared molecules, and identified patterns of miRNAs expression (e.g., from a patient fluid, e.g., blood, sample) may be used to determine the presence or state to help define a specific malady (disease), and/or a response to medication, for example as a companion diagnostic.

In addition, the methods described herein may also be used to help treat patients by indicating the need for and/or monitoring the use of miRNA mimics (e.g., overexpression) and inhibitors/traps (e.g., silencing). The method and apparatuses described herein could provide an easy to use, decentralized, accurate and affordable approach for treating such patients. In particular, the methods and apparatuses described herein allow the specific and rapid detection of a plurality of miRNAs of clinical relevance (whose distinct combinations may point towards specific type of cancer and even stage of the cancer type among other disorders).

As described in detail above, these assays are enzymatic and produce optical (e.g., fluorescent, turbidometric, etc.) signal only in presence of the target miRNA in the studied sample. These techniques have been technically (using synthetic RNA oligos mimicking miRNAs) and biologically (using total RNA from tissue and plasma of disease model mice VS healthy mice as well as healthy human plasma samples) validated to indicate the specificity and sensitivity of the assay by comparison to gold standard qPCR protocols. The method and apparatuses described herein may detect down to 1 fmol, even high amol range, which is a realistic representation of amounts of blood circulating miRNAs. Negative controls with wrong miRNA sequence as well as no input give no signal. For example, the systems described herein have proven able to detect miR-1 in muscle tissue total RNA only and not in liver nor brain, as expected. Similarly, liver specific miR-122 and neuron specific miR-124 were only detected in corresponding tissues and nowhere else. Further, the systems and apparatuses described herein were able to clearly differentiate model mice with Hepatocellular carcinoma from health by looking at both liver tissue miRNA profile and their plasma miRNAs. miR-17, a known oncomir, only resulting in signal from diseased mice.

### Additional Variations

A variety of compositions and methods for analysis if miRNA are described herein. Some variations of the methods described herein are directed to substantially simultaneously analysis of a plurality of miRNAs, including multiplex detection (e.g., by ligation) and amplification of a plurality of miRNAs. While there are examples of multiplex and simultaneous detection of nucleic acids, because of their size and chemical compositions, miRNAs have proved challenging to investigate. Accordingly, the present disclosure is directed to the use of a variety of methods that allow the simultaneous or multiplexed amplification and detection of miRNAs. Although LAMP amplification is the preferred method described in detail above, it is not the exclusive method. For example, detection may occur by any of a number of methods, including but not limited to placement on an ordered or random array, analysis of a multi-well plate, FACS, electrophoretic, spectrophotometric, colorimetric analysis, and the like.

Embodiments of the present disclosure, therefore, find use in detecting miRNAs or other small target polynucleotides and allow for analysis and identification of target polynucleotides from patients or subjects. As discussed above, fluctuations in small target polynucleotides, such as miRNAs, may be indicative of a variety of disorders as described herein and therefore, the present disclosure provides methods of predicting or diagnosing disorders, physiological or pathophysiological conditions associated with altered expression of target polynucleotides, such as miRNAs.

The methods described herein include distributing small target polynucleotides, such as but not limited to RNA, small RNA, miRNA or cDNA, or long non-coding RNA obtained from a biological sample into a plurality of discrete reaction wells. As appreciated by one of ordinary skill in the art, methods of isolating RNA and cDNA may be conventional. Generally the number of individual assays is determined by the size of the microtiter plate used; thus, 96 well, 384 well and 1536 well microtiter plates, and the like may be used in the apparatuses described herein, although as will be appreciated by those in the art, not each microtiter well need be used. It should be noted that some wells may comprise the same reagents as other wells so as to provide duplicates or triplicates, and the like, of the assays performed. As will be appreciated by those in the art, there are a variety of ways to configure the system.

By “biological sample” is meant any bodily fluids (including, but not limited to, blood, urine, serum, lymph, saliva, anal and vaginal secretions, perspiration and semen, of virtually any organism, with mammalian samples being preferred and human samples being particularly preferred); biopsy/tissue material; environmental samples (including, but not limited to, air, agricultural, water and soil samples); biological warfare agent samples; research samples; purified samples, such as purified genomic DNA, RNA, proteins, etc.; raw samples (bacteria, virus, genomic DNA, etc.). As will be appreciated by those in the art, virtually any experimental manipulation may have been done on the sample.

In some variations, each of the wells of the substrate, e.g. micro-titer well or multiwell plate, may comprise a probe or primer specific for a particular target polynucleotide. By target polynucleotide is meant a small RNA, such as but not limited to a miRNA, circular RNAs (circRNA), short interfering RNAs (siRNAs), extracellular RNAs (exRNAs), piwi interacting RNAs (piRNAs), small nucleolar RNAs (snoRNAs), small nuclear RNAs (snRNAs), miscellaneous other RNA (miscRNA) or long non-coding RNA (lncRNA). As noted above, there may be redundancy in the system, so that some wells are designed to contain the same probe or primer as other wells. Alternatively, some wells, may not contain any probe or primer. A primer, when present, is designed to either hybridize to a target polynucleotide or a sequence complementary to a specific target polynucleotide and serve as a primer for ligation, amplification, extension, polymerization or other enzymatic amplification assay.

In some embodiments, the probe or primer nucleic acid serves as a capture probe to hybridize to the target polynucleotide or target polynucleotide-mediated amplification product and retain it in the particular well. As is known in the art, other reagents to support the desired enzymatic, e.g. amplification reaction will also be in the wells of the multi-well plate or similar substrate, such as, but not limited to microfluidic plate, flow cell or lateral flow strip or substrates comprising combinations of these.

By “nucleic acid” or “oligonucleotide” or grammatical equivalents herein means at least two nucleotides covalently linked together. A nucleic acid of the present disclosure will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of labels, or to increase the stability and half-life of such molecules in physiological environments.

As will be appreciated by those in the art, all of these nucleic acid analogs may find use in the present disclosure. In addition, mixtures of naturally occurring nucleic acids and analogs can be made. Alternatively, mixtures of different nucleic acid analogs, and mixtures of naturally occurring nucleic acids and analogs may be made.

Particularly preferred are peptide nucleic acids (PNA), which include peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched base pairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. A preferred embodiment utilizes isocytosine and isoguanine in nucleic acids designed to be complementary to other probes, rather than target sequences, as this reduces non-specific hybridization, as is generally described in U.S. Pat. No. 5,681,702. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

As descried above, an additional component in the assay may be an enzyme for amplification of the target polynucleotide, such as a miRNA, or probes/primers to which the target polynucleotide, such as a miRNA, hybridizes. By this is meant an enzyme that will extend a sequence by the addition of NTPs. As is well known in the art, there are a wide variety of suitable extension enzymes, of which polymerases (both RNA and DNA, depending on the composition of the target sequence, primer and probe) are preferred. Some polymerases are those that lack strand displacement activity, such that they will be capable of adding only the necessary bases at the end of the probe, without further extending the probe to include nucleotides that are complementary to a targeting domain and thus preventing circularization. Suitable polymerases include, but are not limited to, both DNA and RNA polymerases, including the Klenow fragment of DNA polymerase I, SEQUENASE 1.0 and SEQUENASE 2.0 (U.S. Biochemical), T5 DNA polymerase, Phi29 DNA polymerase and various RNA polymerases such as from Thermus sp., or Q beta replicase from bacteriophage, also SP6, T3, T4 and T7 RNA polymerases can be used, among others.

Other polymerases are those that are essentially devoid of a 5′ to 3′ exonuclease activity, so as to assure that the probe will not be extended past the 5′ end of the probe. Exemplary enzymes lacking 5′ to 3′ exonuclease activity include the Klenow fragment of the DNA Polymerase and the Stoffel fragment of DNAPTaq Polymerase. For example, the Stoffel fragment of Taq DNA polymerase lacks 5′ to 3′ exonuclease activity due to genetic manipulations, which result in the production of a truncated protein lacking the N-terminal 289 amino acids. (See e.g., Lawyer et al., J. Biol. Chem., 264:6427-6437 [1989]; and Lawyer et al., PCR Meth. Appl., 2:275-287 [1993]). Analogous mutant polymerases have been generated for polymerases derived from T. maritima, Tsps17, TZ05, Tth and Taf.

Other polymerases are those that lack a 3′ to 5′ exonuclease activity, which is commonly referred to as a proof-reading activity, and which removes bases which are mismatched at the 3′ end of a primer-template duplex. Although the presence of 3′ to 5′ exonuclease activity provides increased fidelity in the strand synthesized, the 3′ to 5′ exonuclease activity found in thermostable DNA polymerases such as Tma (including mutant forms of Tma that lack 5′ to 3′ exonuclease activity) also degrades single-stranded DNA such as the primers used in the PCR, single-stranded templates and single-stranded PCR products. The integrity of the 3′ end of an oligonucleotide primer used in a primer extension process is critical as it is from this terminus that extension of the nascent strand begins. Degradation of the 3′ end leads to a shortened oligonucleotide, which in turn results in a loss of specificity in the priming reaction (i.e., the shorter the primer the more likely it becomes that spurious or non-specific priming will occur).

Other polymerases are thermostable polymerases. For the purposes of this disclosure, a heat resistant enzyme is defined as any enzyme that retains most of its activity after one hour at 40° C. under optimal conditions. Examples of thermostable polymerase that lack both 5′ to 3′ exonuclease and 3′ to 5′ exonuclease include Stoffel fragment of Taq DNA polymerase. This polymerase lacks the 5′ to 3′ exonuclease activity due to genetic manipulation and no 3′ to 5′ activity is present as Taq polymerase is naturally lacking in 3′ to 5′ exonuclease activity. Tth DNA polymerase is derived from Thermus thermophilus, and is available form Epicentre Technologies, Molecular Biology Resource Inc., or Perkin-Elmer Corp. Other useful DNA polymerases which lack 3′ exonuclease activity include a Vent[R](exo-), available from New England Biolabs, Inc., (purified from strains of E. coli that carry a DNA polymerase gene from the archaebacterium Thermococcus litoralis), and Hot Tub DNA polymerase derived from Thermus flavus and available from Amersham Corporation.

Other enzymes that are thermostable and deprived of 5′ to 3′ exonuclease activity and of 3′ to 5′ exonuclease activity include AmpliTaq Gold. Other DNA polymerases, which are at least substantially equivalent, may be used like other N-terminally truncated Thermus aquaticus (Taq) DNA polymerase I. The polymerase named KlenTaq I and KlenTaq LA are quite suitable for that purpose. Of course, any other polymerase having these characteristics can also be.

Other polymerases include those that have strand displacement activity. In one embodiment the enzymes are Bst polymerase and the warm-start Bst 2.0 polymerase from NEB or Lucigen's polymerase called OmniAmp.

There are a number of amplification reactions that may be used in the methods disclosed here. As such, the disclosure provides compositions and methods for amplification and/or detection (and optionally quantification) of products of nucleic acid amplification reactions. Suitable amplification methods include both target amplification and signal amplification. Target amplification involves the amplification (i.e. replication) of the target sequence to be detected, resulting in a significant increase in the number of target molecules. Target amplification strategies include but are not limited to the polymerase chain reaction (PCR), strand displacement amplification (SDA), and nucleic acid sequence based amplification (NASBA).

Alternatively, rather than amplify the target, alternate techniques use the target as a template to replicate a signaling probe, allowing a small number of target molecules to result in a large number of signaling probes, that then can be detected. Signal amplification strategies include the ligase chain reaction (LCR), cycling probe technology (CPT), invasive cleavage techniques such as Invader™ technology, Q-Beta replicase (QβR) technology, and the use of “amplification probes” such as “branched DNA” that result in multiple label probes binding to a single target sequence.

All of these methods require a primer nucleic acid (including nucleic acid analogs) that is hybridized to a target sequence to form a hybridization complex, and an enzyme is added that in some way modifies the primer to form a modified primer. For example, PCR generally requires two primers, dNTPs and a DNA polymerase; LCR requires two primers that adjacently hybridize to the target sequence and a ligase; CPT requires one cleavable primer and a cleaving enzyme; invasive cleavage requires two primers and a cleavage enzyme; etc. Thus, in general, a target polynucleotide, such as a small RNA, such as but not limited to miRNA, is added to a reaction mixture that comprises the necessary amplification components, and a modified primer is formed. Or, when the target polynucleotide serves as the primer for extension, a displaced signaling probe is produced, for instance in the LAMP assay.

In the loop-mediated amplification method (LAMP), shown in FIG. 5, first probes for specific target polynucleotides, such as miRNAs, are distributed in different reaction wells in a substrate, e.g. a 96-well plate. The miRNA, e.g. target polynucleotide, hybridizes to a first complementary region at the 3′ end of the first probe. The first probe also contains a second region complementary to a label probe and the reaction mixture contains a label probe with a first portion complementary the second region of the first probe and a second portion that is not complementary to the label probe. Upon hybridization of the miRNA from the sample with the first probe, a polymerase catalyzes the addition of nucleotides to the 3′ terminus of the miRNA that are complementary to the first probe sequence. This results in displacement of the second probe from the hybrid. Including a second primer that is complementary to the 3′ terminus of the newly synthesized and released probe allows for exponential amplification of the second probe which may be detected either by labels attached to the probe, such as but not limited to magnetic, fluorescent, and/or enzymatic labels, or by detection of double-stranded DNA, for instance by a dye, such as, but not limited to SYBR green, or by fluorescent metal indicators Tomita et al Nature Protocols 2008 (3) 877-882 or colorimetric metal indicators Goto et al Biotechniques 2009 (46) 167-172, both of which are incorporated herein by reference, and the like. LAMP assays may have different configurations, for instance as described in Nakamura et al. Clinica Chimica Acta 411 (2010) 568-573, Jia et al. Angew. Chem. Int. Ed. 2010, 49, 5498-5501, Liu et al. Anal. Chem. 2012, 84, 5165-5169, which are expressly incorporated herein by reference.

Accordingly, the reaction starts with the addition of a probe or primer nucleic acid to the target sequence which forms a hybridization complex. Once the hybridization complex between the probe or primer and the target sequence has been formed, an enzyme, sometimes termed an “amplification enzyme”, is used to modify the primer, which in the case of the LAMP assay or similar assays may be the target polynucleotide, such as a miRNA. As for all the methods outlined herein, the enzymes may be added at any point during the assay, either prior to, during, or after the addition of the primers. The identity of the enzyme will depend on the amplification technique used, as is more fully outlined below. Similarly, the modification will depend on the amplification technique, as outlined below.

Once the enzyme has modified the primer to form a modified primer, the hybridization complex is disassociated. In one aspect, dissociation is by modification of the assay conditions. In another aspect, the modified primer no longer hybridizes to the target nucleic acid and dissociates or is forced to dissociate by strand displacement. Either one or both of these aspects can be employed in signal and target amplification reactions as described below. Generally, the amplification steps are repeated for a period of time to allow a number of cycles, depending on the number of copies of the original target sequence and the sensitivity of detection, with cycles ranging from 1 to thousands, with from 10 to 100 cycles being preferred and from 20 to 50 cycles being especially preferred. When linear strand displacement amplification is used cycle numbers can reach thousands to millions.

Amplification techniques that find use herein and are well known in the art include, but are not limited to polymerase chain reaction (PCR), including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”, “panhandle PCR”, and “PCR select cDNA subtraction”, “allele-specific PCR”, among others. In some embodiments, PCR is not preferred.

In one embodiment, the target amplification technique is SDA. Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby expressly incorporated by reference in their entirety.

In general, SDA may be described as follows. A single stranded target polynucleotide, such as a miRNA, is contacted with an SDA primer. An “SDA primer” generally has a length of 25-100 nucleotides, with SDA primers of approximately 35 nucleotides being preferred. An SDA primer is substantially complementary to a region at the 3′ end of the target sequence, and the primer has a sequence at its 5′ end (outside of the region that is complementary to the target) that is a recognition sequence for a restriction endonuclease, sometimes referred to herein as a “nicking enzyme” or a “nicking endonuclease”, as outlined below. The SDA primer then hybridizes to the target sequence. The SDA reaction mixture also contains a polymerase (an “SDA polymerase”, as outlined below) and a mixture of all four deoxynucleoside-triphosphates (also called deoxynucleotides or dNTPs, i.e. dATP, dTTP, dCTP and dGTP), at least one species of which is a substituted or modified dNTP; thus, the SDA primer is modified, i.e. extended, to form a modified primer, sometimes referred to herein as a “newly synthesized strand”. The substituted dNTP is modified such that it will inhibit cleavage in the strand containing the substituted dNTP but will not inhibit cleavage on the other strand. Examples of suitable substituted dNTPs include, but are not limited, 2′ deoxyadenosine 5′-O-(1-thiotriphosphate), 5-methyldeoxycytidine 5′-triphosphate, 2′-deoxyuridine 5′-triphosphate, and 7-deaza-2′-deoxyguanosine 5′-triphosphate. In addition, the substitution of the dNTP may occur after incorporation into a newly synthesized strand; for example, a methylase may be used to add methyl groups to the synthesized strand. In addition, if all the nucleotides are substituted, the polymerase may have 5′! 3′ exonuclease activity. However, if less than all the nucleotides are substituted, the polymerase preferably lacks 5′! 3′ exonuclease activity.

As will be appreciated by those in the art, the recognition site/endonuclease pair can be any of a wide variety of known combinations. The endonuclease is chosen to cleave a strand either at the recognition site, or either 3′ or 5′ to it, without cleaving the complementary sequence, either because the enzyme only cleaves one strand or because of the incorporation of the substituted nucleotides. Suitable recognition site/endonuclease pairs are well known in the art; suitable endonucleases include, but are not limited to, HincII, HindII, AvaI, Fnu4HI, TthIIII, NclI, BstXI, BamHI, etc. A chart depicting suitable enzymes, and their corresponding recognition sites and the modified dNTP to use is found in U.S. Pat. No. 5,455,166, hereby expressly incorporated by reference.

Once nicked, a polymerase (an “SDA polymerase”) is used to extend the newly nicked strand, 5′! 3′, thereby creating another newly synthesized strand. The polymerase chosen should be able to initiate 5′! 3′ polymerization at a nick site, should also displace the polymerized strand downstream from the nick, and should lack 5′! 3′ exonuclease activity (this may be additionally accomplished by the addition of a blocking agent). Thus, suitable polymerases in SDA include, but are not limited to, the Klenow fragment of DNA polymerase I, SEQUENASE 1.0 and SEQUENASE 2.0 (U.S. Biochemical), T5 DNA polymerase and Phi29 DNA polymerase.

Accordingly, the SDA reaction requires, in no particular order, an SDA primer, an SDA polymerase, a nicking endonuclease, and dNTPs, at least one species of which is modified.

In general, SDA does not require thermocycling. The temperature of the reaction is generally set to be high enough to prevent non-specific hybridization but low enough to allow specific hybridization; this is generally from about 37 C to about 42 C, depending on the enzymes.

In one embodiment, the target amplification technique is nucleic acid sequence based amplification (NASBA). NASBA is generally described in U.S. Pat. No. 5,409,818; Sooknanan et al., Nucleic Acid Sequence-Based Amplification, Ch. 12 (pp. 261-285) of Molecular Methods for Virus Detection, Academic Press, 1995; and “Profiting from Gene-based Diagnostics”, CTB International Publishing Inc., N.J., 1996, all of which are incorporated by reference. NASBA is very similar to both TMA and QBR. Transcription mediated amplification (TMA) is generally described in U.S. Pat. Nos. 5,399,491, 5,888,779, 5,705,365, 5,710,029, all of which are incorporated by reference. The main difference between NASBA and TMA is that NASBA utilizes the addition of RNAse H to effect RNA degradation, and TMA relies on inherent RNAse H activity of the reverse transcriptase.

In general, these techniques may be described as follows. A single stranded target polynucleotide, such as but not limited to a miRNA, is contacted with a first primer, generally referred to herein as a “NASBA primer” (although “TMA primer” is also suitable) and the reaction performed as is known in the art.

In one embodiment the signal amplification technique is RCA, as described in FIG. 6. Rolling-circle amplification is generally described in Baner et al. (1998) Nuc. Acids Res. 26:5073-5078; Barany, F. (1991) Proc. Natl. Acad. Sci. USA 88:189-193; and Lizardi et al. (1998) Nat. Genet. 19:225-232, all of which are incorporated by reference in their entirety.

In general, RCA may be described in two ways. First, as is outlined in more detail below, a single probe is hybridized with a target polynucleotide, such as a miRNA. The probe is circularized while hybridized to the target nucleic acid, or a circular primer is added to the ligated target nucleic acid complex. Addition of a polymerase results in extension of the circular probe. However, since the probe has no terminus, the polymerase continues to extend the probe repeatedly. Thus results in amplification of the circular probe. Additional configurations of RCA are described in more detail in Cheng et al. Angew. Chem. Int. Ed. 2009, 48, 3268-3272, which is expressly incorporated herein by reference.

While in some embodiments thermal cycling is useful in amplification, preferred embodiments herein rely on isothermal amplification techniques described herein and as are known in the art. For instance, duplex-specific nuclease signal amplification assays as described in Yin et al. J. American Chem. Soc. 2012, 134, 5064-5067 find use in the methods disclosed herein. Target-triggered isothermal exponential amplification reactions using DNA-scaffolded silver nanoclusters, as disclosed in Zhang et al. Analyst, 2013, 138, 4812, also find use in the methods disclosed herein. These references are expressly incorporated herein by reference. Additional, amplification assays that find use in the methods disclosed herein include those described in Asiello and Baeumner, Lab Chip, 2011, 11, 1420, incorporated herein by reference.

Once the amplicons or enzymatic products are produced, their presence must be detected. Different labels may be used. By “detection label” or “detectable label” herein is meant a moiety that allows detection. This may be a primary label or a secondary label. Accordingly, detection labels may be primary labels (i.e. directly detectable) or secondary labels (indirectly detectable).

In a preferred embodiment, the detection label is a primary label. A primary label is one that can be directly detected, such as a fluorophore. In general, labels fall into three classes: a) isotopic labels, which may be radioactive or heavy isotopes; b) magnetic, electrical, thermal labels; and c) colored or luminescent dyes. Labels can also include enzymes (horseradish peroxidase, etc.) and magnetic particles. Preferred labels include chromophores or phosphors but are preferably fluorescent dyes. Suitable dyes for use herein may include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue™, Texas Red, Cy dyes (Cy3, Cy5, etc.), alexa dyes, phycoerythin, bodipy, and others described in the 6th Edition of the Molecular Probes Handbook by Richard P. Haugland, hereby expressly incorporated by reference. Carbon nanotubes also find use as detection agents as outlined in Wang et al. Analyst, 2012, 137, 3667. Or fluorescent metal indicators such as calcein Tomita et al Nature Protocols 2008 (3) 877-882 or colorimetric metal indicators such as Hydroxy Napthol Blue (HNB) Goto et al Biotechniques 2009 (46) 167-172, both of which are incorporated herein by reference, find use in embodiments described herein.

In a preferred embodiment, a secondary detectable label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels find particular use in systems requiring separation of labeled and unlabeled probes, such as SBE, OLA, invasive cleavage reactions, etc.; in addition, these techniques may be used with many of the other techniques described herein. Secondary labels include, but are not limited to, one of a binding partner pair; chemically modifiable moieties; nuclease inhibitors, enzymes such as horseradish peroxidase, alkaline phosphatases, luciferases, etc.

In a preferred embodiment, the secondary label is a binding partner pair. For example, the label may be a hapten or antigen, which will bind its binding partner. In a preferred embodiment, the binding partner can be attached to a solid support to allow separation of extended and non-extended primers. For example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. In general, the smaller of the pair is attached to the NTP for incorporation into the primer. Preferred binding partner pairs include, but are not limited to, biotin (or imino-biotin) and streptavidin, digeoxinin and Abs, and Prolinx™ reagents (see www.prolinxinc.com/ie4/home.hmtl).

In a preferred embodiment, the binding partner pair comprises biotin or imino-biotin and streptavidin. Imino-biotin is particularly preferred as imino-biotin disassociates from streptavidin in pH 4.0 buffer while biotin requires harsh denaturants (e.g. 6 M guanidinium HCl, pH 1.5 or 90% formamide at 95° C.).

In a preferred embodiment, the binding partner pair comprises a primary detection label (for example, attached to the NTP and therefore to the extended primer) and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10-4-10-6 M-1, with less than about 10-5 to 10-9 M-1 being preferred and less than about 10-7-10-9 M-1 being particularly preferred.

In a preferred embodiment, the secondary label is a chemically modifiable moiety. In this embodiment, labels comprising reactive functional groups are incorporated into the nucleic acid. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo- or hetero-bifunctional linkers as are well known (see 1994 Pierce Chemical Company catalog, technical section on cross-linkers, pages 155-200, incorporated herein by reference).

However, in this embodiment, the label is a secondary label, a purification tag, that can be used to capture the sequence comprising the tag onto a second solid support surface.

The addition of the polymerase and the labeled dNTP are done under conditions to allow the formation of a modified first probe. The modified first probe is then added to a second solid support using the purification tag as outlined herein.

In one embodiment the assay is run in microtiter plates as known in the art. The substrate or microtiter plate is placed in a device for running the assay and may optionally contain a detector. The detector may be integrated or removable. FIG. 11D illustrates another embodiment of a device (plate reader), similar to that described above (and shown in FIGS. 11A-11C). In FIG. 11D, device 100 is a device for detecting a target polynucleotide, such as a miRNA profile in a biologic fluid, in accordance with a first exemplary embodiment of the present disclosure. The device 100 is capable of detecting small RNAs, for example, by utilizing a loop mediated isothermal amplification (LAMP) based molecular screening test. The device 100 may contain a thermoblock 110 on which the LAMP or other amplification reaction can take place. As described herein the substrate in which the reactions occur may be a PCR plate 120 or microreactor chambers of a microfluidic chip. Also, as described herein, a target polynucleotide specific probe or molecular trap for a specific target polynucleotide, such as a small RNA is found in each well or microreactor.

In the presence of the target polynucleotide, such as a small RNA, in the tested biological sample, the enzymatic assay, such as a LAMP assay, produces sufficient fluorescence or color change that is recordable through the use of a detector, such as a camera. In some embodiments, the camera may be integrated with the device or may be removable. In some embodiments the detector is capable of wireless or wired transmission of signals to a second location, which may house computers storing data and algorithms for analyzing the test results. In some embodiments, the detector may be a mobile phone or smart phone 130. Fluorescence/color detection/spectophotometry may be performed in real time during the course of the reaction, which can last anywhere between 1-120 minutes, from 5-100 minutes, from 30-90 min, or from 40-70 minutes. As such, the time from initiation of the assay to detection of results may be less than 120 minutes, less than 100 minutes, less than 90 minutes, less than 75 minutes, less than 60 minutes, less than 45 minutes, less than 30 minutes or less than 15 minutes. Accordingly, identification of samples having a positive result and therefore capable of diagnosing physiological or pathophysiological conditions may similarly be less than 120 minutes, less than 100 minutes, less than 90 minutes, less than 75 minutes, less than 60 minutes, less than 45 minutes, less than 30 minutes or less than 15 minutes.

The recorded fluorescence/color patterns displayed on the PCR plate or microfluidic chip are uploaded to a server by the detector and/or assay device (e.g., by the smart phone 130 via cellular, wifi or any other communication protocol) and analyzed. Analysis may be performed by any combination of software and/or hardware tools contained on the detector 130, the server and/or any other electronically accessible computer. Algorithms provided by this disclosure may be implemented in software, which may be executed by computer hardware to perform the analysis and other functions provided herein.

Specific target polynucleotide profiles are assigned to each biological sample and computationally linked to specific fluorescence/color patterns. Collection of sufficient fluorescence patterns from as many biological samples as possible will enable a statistically significant assignment of a link between pathophysiological status and a pattern of fluorescence, in other words a target polynucleotide such as a small RNA, such as a miRNA, profile characteristic of disease. Thus, in one embodiment the method as described herein is used to identify and screen for miRNA molecules characteristic of a particular sample type, e.g. cell type, or disease state, e.g. cancerous cell. Alternatively, the method is used to examine samples and compare the miRNA profile with that of known profiles, or profiles generated by the methods and systems disclosed herein, to predict the likelihood of disease occurrence, prognosis or diagnosis.

Accordingly, embodiments of the present disclosure include identification or analysis of target polynucleotide, such as a small RNA, such as a miRNA, expression profiles as an indicator of the likelihood of disease of the patient or subject from which the samples are derived. In some embodiments, the disease is a neoplasia/cancer, immunopathological disorder, infectious disease, metabolic disease, inflammatory disorder, neurological disorder, tissue/cell injury, hemodynamic disorders, environmental diseases, genetic disorders.

As such, the disclosure herein provides any necessary servers, computers, memory and the like. The system for analyzing target polynucleotide expression profiles, such as miRNA expression profiles, can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

In an embodiment, the present disclosure provides the system described above that stores software for comparing expression profiles received from the detector or device with stored expression profiles correlating the expression profiles with disease or other physiological or pathophysiological conditions. The memory also may comprise software to develop and store expression profiles for newly generated or confirmed by the miRNA expression analyses described herein. As such, target polynucleotide expression patterns may be compared with data from public databases, the literature or proprietary databases, including databases of expression profiles generated by the methods outlined herein. The system also includes software for encrypting and decrypting data sent from the detection device or assay device to the computer/server for analysis. In addition, the system includes secure login features so that only those with appropriate access may utilize or log in to the system.

As described above, a computer comprising software and memory comprising a database of miRNA profiles and software to perform the analysis analyzes the detected signal from the assay. In some embodiments the computer is local to the assay device but in other embodiments the computer or server is removed from the assay device. Regardless the location of the computer or server, the signal or data must be transmitted to it. Transfer of a disk, or thumb drive containing the data between the devices may accomplish this. Alternatively the data is transmitted wirelessly to a remote location. For instance, FIG. 25 is a diagram illustrating a method of detecting and analyzing a miRNA profile in a biologic fluid, in accordance with an embodiment of the present disclosure. As shown at block 202, RNA may be extracted from a blood sample from a patient (in some variations a separate extraction step is not necessary). The RNA may be extracted from the sample using any known technique. At block 204, the plate or microfluidic chip may be prepared using the extracted RNA. The plate may be loaded into the device as shown in block 206, and the miRNA profile may be detected, e.g., through fluorescence patterns emitted utilizing the LAMP method as described above. The detected fluorescence patterns (which may be detected as image data captured by a smart phone 130 or other detector/camera) and may be transmitted to a server that may analyze the received data, e.g., by correlating the received fluorescence patterns with fluorescence patterns having known associations with one or more pathophysiological conditions stored in a database accessible to the server.

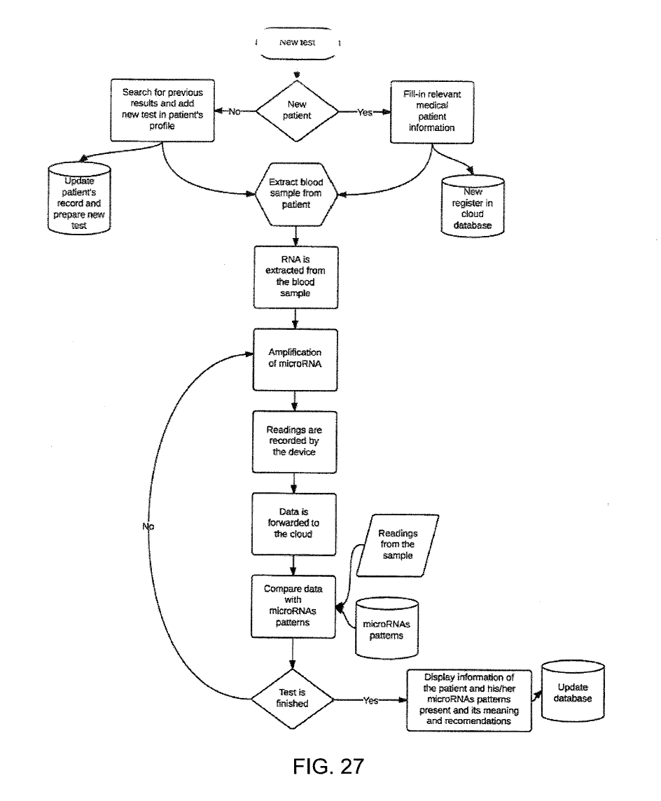

FIG. 26A-26C illustrates associations between micro-RNAs and certain types of diseases (including cancers). The results of an analysis (which may include a specific identification of the micro-RNA profile and/or an indication of the presence or absence of one or more diseases or health-related conditions) may be communicated from the server to a user, such as a physician, patient, insurer, hospital and the like. A profile, such as the one shown in FIG. 26B, indicating the presence of a sub-set of known microRNAs may be matched to a database of associated or suspected associations, as shown. The profile may be sent via e-mail or wirelessly to any desired receiving device, such as a computer having sufficient access to the results in the server and/or a smart phone. FIG. 27 provides an exemplary flowchart demonstrating an embodiment of a system described herein.

Accordingly, the present disclosure provides a method for diagnosing physiological or pathophysiological conditions by detecting specific miRNAs characteristic of a particular physiological or pathophysiological condition, such as cancer, different stages of cancer, inflammatory disorders, neurological disorders and the like.

As for additional details pertinent to the present invention, materials and manufacturing techniques may be employed as within the level of those with skill in the relevant art. The same may hold true with respect to method-based aspects described herein in terms of additional acts commonly or logically employed. Also, it is contemplated that any optional feature of the inventive variations described may be set forth and claimed independently, or in combination with any one or more of the features described herein. It is further noted that claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of detecting a plurality of microRNAs in parallel from a patient sample containing microRNA using a multiplexed ligation and detection technique, the method comprising:
combining the patient sample with a first mixture to form a multiplexing mixture comprising a plurality of pairs of donor template and acceptor template, wherein each pair of donor template and acceptor template is specific to a target microRNA of the plurality of microRNAs because a 5′ end of a donor template and a 3′ end of an acceptor template in each pair comprise regions that are complimentary to adjacent portions of the target microRNA and further wherein one or both of the 3′ end of the donor template and the 5′ end of the acceptor template comprises one or more nucleotide sequence that is specific to the pair of donor and acceptor template;
heating the multiplexing mixture to denature the microRNA, and cooling the multiplexing mixture to anneal pairs of donor and acceptor template to specific target microRNA if the target microRNA is present in the multiplexing mixture;
ligating the annealed pairs of donor template and acceptor template using a ligase to form a template specific to target microRNA;
inactivating the ligase;
placing a portion of the multiplexing mixture into each of a plurality of wells;
performing, in parallel, loop-mediated isothermal amplification of template specific to a different target microRNA in each of the plurality of wells, wherein each well is associated with one specific target microRNA from the plurality of microRNAs and wherein each well comprises a polymerase having strand displacement activity and primers for the loop mediated amplification, wherein one or more of the primers for loop mediated amplification includes the nucleotide sequence that is specific to the pair of donor and acceptor template or a complement to the nucleotide sequence that is specific to the pair of donor and acceptor template and therefore specific to one target microRNA of the plurality of microRNAs.

2. A method of detecting a plurality of microRNAs in parallel from a patient sample containing microRNA using a multiplexed ligation and detection technique, the method comprising:
combining the patient sample with a first mixture to form a multiplexing mixture comprising a plurality of pairs of donor template and acceptor template, wherein a 5′ end of the donor template and a 3′ end of the acceptor template of each pair comprise regions that are complimentary to adjacent portions of one specific target microRNA from the plurality of microRNAs, and further wherein each acceptor template comprises a B3 region at a 5′ end of the acceptor template, a B2 region 3′ to the B3 region, and a B1 region 3′ to the B2 region, wherein each donor template comprises an F3c region at the 3′ end of the donor template, an F2c region 5′ to the F3c region, and an F1c region 5′ to the F2c region, and wherein each pair of donor and acceptor templates includes a unique sequence that is different from the other pairs for at least one of: the B3 region, the B2 region, the B1 region, the F3c region, the F2c region, and the F1c region;
heating the multiplexing mixture to denature the microRNA, and cooling the multiplexing mixture to anneal the pair of donor and acceptor template to the specific target microRNA if that specific target microRNA is present in the multiplexing mixture;
ligating the annealed pairs of donor template and acceptor template using a ligase to form a template specific to target microRNA;
inactivating the ligase;
placing a portion of the multiplexing mixture into each of a plurality of wells;
performing loop-mediated isothermal amplification of each of the plurality of wells in parallel, wherein each well is associated with one specific target microRNA from the plurality of microRNAs and comprises a combination of primers that complement or include the unique sequence that is different from the other pairs of the plurality of pairs of donor template and acceptor template for at least one of: the B3 region, the B2 region, the B1 region, the F3c region, the F2c region, and the F1c region of the template specific to target microRNA.

3. The method of claim 1 or 2, wherein for each specific target microRNA, the donor template in one of the plurality of pairs comprises a reverse compliment at its 5′ end of a first portion of the specific target microRNA sequence and wherein the acceptor template in each pair comprises a reverse compliment at its 3′ end of a second portion of the specific target microRNA sequence.

4. The method of claim 1 or 2, wherein the donor template of each pair is modified to have a phosphate group at its 5′ end.

5. The method of claim 1 or 2, wherein the donor template and the acceptor template of each pair are oligonucleotides of less than 150 base pairs each.

6. The method of claim 1 or 2, wherein combining comprises combining the patient sample with the first mixture so that there is 10 nM or less of each of donor template and target template.

7. The method of claim 2, wherein each pair of donor and acceptor templates includes a unique sequence for the F2c region, the F1c region or both the F2c region and the F1c region.

8. The method of claim 2, wherein heating comprises heating the multiplexing mixture to between about 70° C. and 99° C. for greater than 1 min.

9. The method of claim 1 or 2, wherein cooling the multiplexing mixture comprises gradually cooling to room temperature.

10. The method of claim 1 or 2, wherein ligating comprises adding less than 4 nM of ligase into the multiplexing mixture.

11. The method of claim 1 or 2, wherein ligating comprises using less than 4 nM of ligase in the presence of MnCl₂ and less than 5 μM ATP in the multiplexing mixture.

12. The method of claim 1 or 2, wherein ligating comprises ligating for between about 10-60 min at between about 20-40° C.

13. The method of claim 1 or 2, wherein inactivating the ligase comprises heating the multiplexing mixture to greater than 60° C. for 10 min or more.

14. The method of claim 1 or 2, wherein performing loop-mediated isothermal amplification comprises amplifying one of the templates specific to target microRNA in each well to indicate the presence of the target microRNA in the patient sample by maintaining the temperature of the well between 60 and 70 degrees in the presence of a forward inner primer (FIP) that hybridizes to the nucleotide sequence that is specific to the pair of donor and acceptor template specific to target microRNA and includes a second region that is identical to a portion of the template specific to target microRNA.

15. The method of claim 14, wherein performing loop-mediated isothermal amplification further comprises amplifying in the presence of a forward outer primer (FOP) that hybridizes to the template specific to target microRNA, a backwards inner primer (BIP) comprising a nucleotide region of the template specific to target microRNA and a second region that hybridizes to the template specific to target microRNA, and a backwards outer primer (BOP) comprising a region of the template specific to target microRNA.

16. The method of claim 2, wherein performing loop-mediated isothermal amplification comprises amplifying one of the templates specific to target microRNA in each well to indicate the presence of the target microRNA in the patient sample by maintaining the temperature of the well between 60 and 70 degrees in the presence of a forward inner primer (FIP) comprising an F2 region that hybridizes to the F2c region of the template specific to target microRNA and the F1c region of the template specific to target microRNA, a forward outer primer (FOP) comprising an F3 region that hybridizes to the F3c region of the template specific to target microRNA, a backwards inner primer (BIP) comprising the B2 region of the template specific to target microRNA and a B1c region that hybridizes to the B1 region of the template specific to target microRNA, and a backwards outer primer (BOP) comprising the B3 region of the template specific to target microRNA, and a polymerase having strand displacement activity.

17. The method of claim 1 or 2 further comprising detecting a visual change in one or more wells indicating the presence of the specific target microRNA associated with that well in the patient sample.

18. The method of claim 1 or 2, further comprising correlating signals corresponding to a visual change in a plurality of the wells with known profiles corresponding to disease states to identify a disease state.

19. The method of claim 1 or 2, further comprising transmitting a signal corresponding to a visual change in plurality of the wells to a remote processor for correlation analysis with known profiles corresponding to disease states.

20. A system for detecting a plurality of microRNAs in parallel from a patient sample containing microRNA using a multiplexed ligation and detection technique, the system comprising:
a first solution mixture comprising a plurality of pairs of donor template and acceptor template, wherein each pair of donor template and acceptor template is specific to a target microRNA of the plurality of microRNAs because a 5′ end of a donor template and a 3′ end of an acceptor template in each pair comprise regions that are complimentary to adjacent portions of the target microRNA and further wherein one or both of the 3′ end of the donor template and the 5′ end of the acceptor template comprises one or more nucleotide sequence that is specific to the pair of donor and acceptor template; and
a multiwell reaction substrate for performing, in parallel, loop-mediated isothermal amplification to detect target microRNA in each of a plurality of wells, wherein each well is associated with one specific target microRNA from the plurality of microRNAs, and wherein each well comprises a plurality of primers for the loop mediated amplification, wherein one or more of the primers for loop mediated amplification within each well includes the nucleotide sequence that is specific to the pair of donor and acceptor template or a complement to the nucleotide sequence that is specific to the pair of donor and acceptor template and therefore specific to one target microRNA of the plurality of microRNAs associated with that well.

21. The system of claim 20, wherein the multiwell reaction substrate further comprises a polymerase having strand displacement activity within each well.

22. The system of claim 20, further comprising a multiwell plate reader for performing, in parallel, loop-mediated isothermal amplification to detect target microRNA in each of a plurality of wells of a multiwell reaction substrate, wherein each well is associated with one specific target microRNA from the plurality of microRNAs, the multiwell plate reader comprising:
thermal control circuitry configured to maintain the plurality of wells at a temperature of between 60-70° C., wherein the control circuitry comprises a board having a plurality of thermal control elements configured to surround individual wells of the multiwell reaction substrate,
one or more light sources configured to illuminate wells of the multiwell reaction substrate,
a plurality of optical detectors, wherein each optical detector is configured to monitor a well of the multiwell reaction substrate, and
a communication module configured to transmit sample data collected from the plurality of optical detectors to a remote processor.

23. The system of claim 20, wherein the first solution mixture is lyophilized.

24. A system for detecting a plurality of microRNAs in parallel from a patient sample containing microRNA using a multiplexed ligation and detection technique, the system comprising:
a first solution mixture comprising a plurality of pairs of donor template and acceptor template, wherein each pair of donor template and acceptor template is specific to a target microRNA of the plurality of microRNAs because a 5′ end of a donor template and a 3′ end of an acceptor template in each pair comprise regions that are complimentary to adjacent portions of the target microRNA and further wherein one or both of the 3′ end of the donor template and the 5′ end of the acceptor template comprises one or more nucleotide sequence that is specific to the pair of donor and acceptor template; and
a multiwell plate reader for performing, in parallel, loop-mediated isothermal amplification to detect target microRNA in each of a plurality of wells of a multiwell reaction substrate, wherein each well is associated with one specific target microRNA from the plurality of microRNAs, the multiwell plate reader comprising:
thermal control circuitry configured to maintain the plurality of wells at a temperature of between 60-70° C., wherein the control circuitry comprises a board having a plurality of thermal control elements configured to surround individual wells of the multiwell reaction substrate,
one or more light sources configured to illuminate wells of the multiwell reaction substrate,
a plurality of optical detectors, wherein each optical detector is configured to monitor a well of the multiwell reaction substrate, and
a communication module configured to transmit sample data collected from the plurality of optical detectors to a remote processor.

25. The system of claim 24, further comprising a multiwell reaction substrate.

26. The system of claim 24, wherein the first solution mixture is lyophilized.

27. The system of claim 24, wherein the communication module is a wireless communication module.

28. The system of claim 24, further comprising a non-transitory computer-readable storage medium storing a set of instructions capable of being executed by a smartphone to control the operation of the multiwell plate reader, and that when executed by the smartphone, causes the smartphone to:
identify and wirelessly communicate with the multiwell plate reader;
associate a multiwell reaction substrate with a patient;
start a detection assay in the multiwell plate reader;
receive optical data from the multiwell plate reader, wherein the optical data comprises optical information from the plurality of optical detectors; and
connect to a remote server to transmit and receive information about the optical data.

29. The system of claim 24, wherein the set of instructions when executed by the smartphone, causes the smartphone to transmit an alert when the detection assay is completed.

30. The system of claim 24, wherein the set of instructions when executed by the smartphone, causes the smartphone to save data for later transmission to the remote server.

31. The system of claim 24, wherein the set of instructions when executed by the smartphone, causes the smartphone to present information about the optical data on a display of the smartphone.

32. The system of claim 24, wherein the set of instructions when executed by the smartphone, causes the smartphone to receive optical data from the multiwell plate reader at periodic intervals for a predetermined period of time.
